(19) United States
(12) Patent Application Publication
(10) Pub. No.: US 2022/0169643 A1
(43) Pub. Date: Jun. 2, 2022

(54) ESTROGEN RECEPTOR DEGRADING PROTACS

(71) Applicant: AstraZeneca AB, Södertälje (SE)

(72) Inventors: Bin YANG, Wilmington, DE (US); Thomas George Christopher HAYHOW, Cambridge (GB); Charlene FALLAN, Cambridge (GB); James Stewart SCOTT, Cambridge (GB); Coura Rosalie DIENE, Cambridge (GB); Bernard Christophe BARLAAM, Cambridge (GB); Johannes Wilhelmus Maria NISSINK, Cambridge (GB)

(21) Appl. No.: 17/442,329

(22) PCT Filed: Mar. 27, 2020
(86) PCT No.: PCT/EP2020/058702

§ 371 Date: Sep. 23, 2021

Related U.S. Application Data
(60) Provisional application No. 62/825,924, filed on Mar. 29, 2019.

(51) Int. Cl. C07D 471/04; C07D 519/00

(52) U.S. Cl. CPC C07D 471/04; C07D 519/00

(57) ABSTRACT

The specification generally relates to compounds of Formula (I): (I) and pharmaceutically acceptable salts thereof, where R¹, R², R³, R⁴, R⁶, R⁷, R⁸, Linker, A, G, D and E have any of the meanings defined herein. This specification also relates to the use of such compounds and pharmaceutically acceptable salts thereof in methods of treatment of the human or animal body, for example in prevention or treatment of cancer. This specification also relates to processes and intermediate compounds involved in the preparation of such compounds and to pharmaceutical compositions containing them.

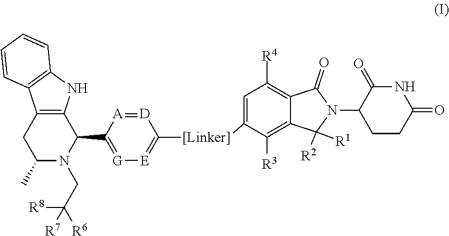

### FIELD

The compounds of the specification have been found to possess potent anti-tumour activity, being useful in inhibiting the uncontrolled cellular proliferation which arises from malignant disease. The compounds of the specification provide an anti-tumour effect by, as a minimum, acting as Proteolysis Targeting Chimeras (PROTACs) to selectively degrade estrogen receptor alpha. For example, the compounds of the specification may exhibit anti-tumour activity via the ability to degrade the estrogen receptor in a number of different breast cancer cell-lines, for example against the MCF-7, CAMA-1, and/or BT474 breast cancer cell-lines. Such compounds may be expected to be more suitable as therapeutic agents, particularly for the treatment of cancer. This specification also relates to processes and intermediate compounds involved in the preparation of said compounds and to pharmaceutical compositions containing them.

### BACKGROUND

Estrogen receptor alpha (ERα, ESR1, NR3 A) and estrogen receptor beta (ERβ, ESR2, NR3b) are steroid hormone receptors which are members of the large nuclear receptor family. Structured similarly to all nuclear receptors, ERα is composed of six functional domains (named A-F) (Dahlman-Wright, et al., Pharmacol. Rev., 2006, 58:773-781) and is classified as a ligand-dependent transcription factor because after its association with the specific ligand, (the female sex steroid hormone 17b estradiol), the complex binds to genomic sequences, named Estrogen Receptor Elements (ERE) and interacts with co-regulators to modulate the transcription of target genes. The ERα gene is located on 6q25.1 and encodes a 595AA protein and multiple isoforms can be produced due to alternative splicing and translational start sites. In addition to the DNA binding domain (Domain C) and the ligand binding domain (Domain E) the receptor contains a N-terminal (A/B) domain, a hinge (D) domain that links the C and E domains and a C-terminal extension (F domain). While the C and E domains of ERα and ERβ are quite conserved (96% and 55% amino acid identity respectively) conservation of the A/B, D and F domains is poor (below 30% amino acid identity). Both receptors are involved in the regulation and development of the female reproductive tract and in addition play roles in the central nervous system, cardiovascular system and in bone metabolism. The genomic action of ERs occurs in the nucleus of the cell when the receptor binds EREs directly (direct activation or classical pathway) or indirectly (indirect activation or non-classical pathway). In the absence of ligand, ERs are associated with heat shock proteins, Hsp90 and Hsp70, and the associated chaperone machinery stabilizes the ligand binding domain (LBD) making it accessible to ligand. Liganded ER dissociates from the heat shock proteins leading to a conformational change in the receptor that allows dimerisation, DNA binding, interaction with co-activators or co-repressors and modulation of target gene expression. In the non-classical pathway, AP-1 and Sp-1 are alternative regulatory DNA sequences used by both isoforms of the receptor to modulate gene expression. In this example, ER does not interact directly with DNA but through associations with other DNA bound transcription factors e.g. c-Jun or c-Fos (Kushner et al., Pure Applied Chemistry 2003, 75:1757-1769). The precise mechanism whereby ER affects gene transcription is poorly understood but appears to be mediated by numerous nuclear factors that are recruited by the DNA bound receptor. The recruitment of co-regulators is primarily mediated by two protein surfaces, AF2 and AF1, which are located in E-domain and the A/B domain respectively. AF1 is regulated by growth factors and its activity depends on the cellular and promoter environment whereas AF2 is entirely dependent on ligand binding for activity. Although the two domains can act independently, maximal ER transcriptional activity is achieved through synergistic interactions via the two domains (Tzukerman, et al., Mol. Endocrinology, 1994, 8:21-30). Although ERs are considered transcription factors they can also act through non-genomic mechanisms as evidenced by rapid ER effects in tissues following estradiol administration in a timescale that is considered too fast for a genomic action. It is still unclear if receptors responsible for the rapid actions of estrogen are the same nuclear ERs or distinct G-protein coupled steroid receptors (Warner, et al., Steroids 2006 71:91-95) but an increasing number of estradiol induced pathways have been identified e.g. MAPK/ERK pathway and activation of endothelial nitric oxide synthase and PI3K/Akt pathway. In addition to ligand dependent pathways, ERα has been shown to have ligand independent activity through AF-1 which has been associated with stimulation of MAPK through growth factor signalling e.g. insulin like growth factor 1 (IGF-1) and epidermal growth factor (EGF). Activity of AF-1 is dependent on phosphorylation of Ser118 and an example of cross-talk between ER and growth factor signalling is the phosphorylation of Ser118 by MAPK in response to growth factors such as IGF-1 and EGF (Kato, et al., Science, 1995, 270:1491-1494).

A large number of structurally distinct compounds have been shown to bind to ER. Some compounds such as endogenous ligand estradiol, act as receptor agonists whereas others competitively inhibit estradiol binding and act as receptor antagonists. These compounds can be divided into 2 classes depending on their functional effects. Selective estrogen receptor modulators (SERMs) such as tamoxifen have the ability to act as both receptor agonists and antagonists depending on the cellular and promoter context as well as the ER isoform targeted. For example tamoxifen acts as an antagonist in breast but acts as a partial agonist in bone, the cardiovascular system and uterus. All SERMs appear to act as AF2 antagonists and derive their partial agonist characteristics through AF1. A second group, fulvestrant being an example, are classified as full antagonists and are capable of blocking estrogen activity via the complete inhibition of AF1 and AF2 domains through induction of a unique conformation change in the ligand binding domain (LBD) on compound binding which results in complete abrogation of the interaction between helix 12 and the remainder of the LBD, blocking co-factor recruitment (Wakeling, et al., Cancer Res., 1991, 51:3867-3873; Pike, et al., Structure, 2001, 9:145-153).

Intracellular levels of ERα are down-regulated in the presence of estradiol through the ubiquitin/proteosome (Ub/26S) pathway. Polyubiquitinylation of liganded ERα is catalysed by at least three enzymes; the ubiquitin-activating enzyme E1 activated ubiquitin is conjugated by E2 conjugating enzyme with lysine residues through an isopeptide bond by E3 ubiquitin ligase and polyubiquitinated ERα is then directed to the proteosome for degradation. Although ER-dependent transcription regulation and proteosome-mediated degradation of ER are linked (Lonard, et al., Mol. Cell, 2000 5:939-948), transcription in itself is not required for ERα degradation and assembly of the transcription initiation complex is sufficient to target ERα for nuclear proteosomal degradation. This estradiol induced degradation process is believed necessary for its ability to rapidly activate transcription in response to requirements for cell proliferation, differentiation and metabolism (Stenoien, et al., Mol. Cell Biol., 2001, 21:4404-4412). Fulvestrant is also classified as a selective estrogen receptor degrader (SERD), a subset of antagonists that can also induce rapid down-regulation of ERα via the 26S proteosomal pathway. In contrast a SERM such as tamoxifen can increase ERα levels although the effect on transcription is similar to that seen for a SERD.

PROTACs are heterobifunctional molecules containing two small molecule binding moieties, joined together by a linker. One of the small molecule ligands is designed to bind with high affinity to a target protein in the cell whilst the other ligand is able to bind with high affinity to an E3 ligase. In the cell, the PROTAC seeks out and selectively binds to the target protein of interest. The PROTAC then recruits a specific E3 ligase to the target protein to form a ternary complex with both the target protein and the E3 ligase held in close proximity. The E3 ligase then recruits an E2 conjugating enzyme to the ternary complex. E2 is then able to ubiquitinate the target protein, labelling an available lysine residue on the protein and then dissociates from the ternary complex. E3 can then recruit additional E2 molecules resulting in poly-ubiquitination of the target protein, labelling the target protein for potential degradation by the cell's proteasome machinery. A PROTAC is then able to dissociate from the target protein and initiate another catalytic cycle. The poly-ubiquitinated target protein is then recognized and degraded by the proteasome. Here the designated PROTACs targeting ER for degradation contain an ER ligand moiety at one end of the linker and an E3 ligase (such as cereblon, CRBN) ligand at the other end. In the cells, the ER PROTAC selectively recruits CRBN E3 ligase to ER and leads to the degradation of ER by the Ub/26S system.

Approximately 70% of breast cancers express ER and/or progesterone receptors implying the hormone dependence of these tumour cells for growth. Other cancers such as ovarian and endometrial are also thought to be dependent on ERα signalling for growth. Therapies for such patients can inhibit ER signalling either by antagonising ligand binding to ER e.g. tamoxifen which is used to treat early and advanced ER positive breast cancer in both pre and post menopausal setting; antagonising and down-regulating ERα e.g. fulvestrant which is used to treat breast cancer in women which have progressed despite therapy with tamoxifen or aromatase inhibitors; or blocking estrogen synthesis e.g. aromatase inhibitors which are used to treat early and advanced ER positive breast cancer. Although these therapies have had an enormously positive impact on breast cancer treatment, a considerable number of patients whose tumours express ER display de novo resistance to existing ER therapies or develop resistance to these therapies over time. Several distinct mechanisms have been described to explain resistance to first-time tamoxifen therapy which mainly involve the switch from tamoxifen acting as an antagonist to an agonist, either through the lower affinity of certain co-factors binding to the tamoxifen-ERα complex being off-set by over-expression of these co-factors, or through the formation of secondary sites that facilitate the interaction of the tamoxifen-ERα complex with co-factors that normally do not bind to the complex. Resistance could therefore arise as a result of the outgrowth of cells expressing specific co-factors that drive the tamoxifen-ERα activity. There is also the possibility that other growth factor signalling pathways directly activate the ER receptor or co-activators to drive cell proliferation independently of ligand signalling.

More recently, mutations in ESR1 have been identified as a possible resistance mechanism in metastatic ER-positive patient derived tumour samples and patient-derived xenograft models (PDX) at frequencies varying from 17-25%. These mutations are predominantly, but not exclusively, in the ligand-binding domain leading to mutated functional proteins; examples of the amino acid changes include Ser463Pro, Val543Glu, Leu536Arg, Tyr537Ser, Tyr537Asn and Asp538Gly, with changes at amino acid 537 and 538 constituting the majority of the changes currently described. These mutations have been undetected previously in the genomes from primary breast samples characterised in the Cancer Genome Atlas database. Of 390 primary breast cancer samples positive for ER expression not a single mutation was detected in ESR1 (Cancer Genome Atlas Network, 2012 Nature 490: 61-70). The ligand binding domain mutations are thought to have developed as a resistance response to aromatase inhibitor endocrine therapies as these mutant receptors show basal transcriptional activity in the absence of estradiol. The crystal structure of ER, mutated at amino acids 537 and 538, showed that both mutants favoured the agonist conformation of ER by shifting the position of helix 12 to allow co-activator recruitment and thereby mimicking agonist activated wild type ER. Published data has shown that endocrine therapies such as tamoxifen and fulvestrant can still bind to ER mutant and inhibit transcriptional activation to some extent and that fulvestrant is capable of degrading Try537Ser but that higher doses may be needed for full receptor inhibition (Toy et al., Nat. Genetics 2013, 45: 1439-1445; Robinson et al., Nat. Genetics 2013, 45: 144601451; Li, S. et al. Cell Rep. 2013, 4, 1116-1130). It is therefore feasible that certain compounds of the Formula (I) or pharmaceutically acceptable salts thereof (as described hereinafter) will be capable of antagonising mutant ER although it is not known at this stage whether ESR1 mutations are associated with an altered clinical outcome.

Regardless of which resistance mechanism or combination of mechanisms takes place, many are still reliant on ER-dependent activities and antagonism or degradation of the receptor offers a way of inhibiting ERα. There is therefore an ongoing need for therapies which selectively degrade estrogen receptor alpha.

### SUMMARY

The compounds of the specification have been found to provide an anti-tumour effect by inducing ER degradation, or as a minimum, acting as ER antagonists. The compounds described herein may provide greater ER degredation compared to fulvestrant and may also provide greater ER degradation compared to oral SERDs. The compounds of the specification may be expected to be suitable as therapeutic agents, particularly for the treatment of cancer.

This specification relates to certain compounds and pharmaceutically acceptable salts thereof that selectively degrade the estrogen receptor and possess anti-cancer activity. This specification also relates to use of said compounds and pharmaceutically acceptable salts thereof in methods of treatment of the human or animal body, for example in prevention or treatment of cancer. This specification also relates to processes and intermediate compounds involved in the preparation of said compounds and to pharmaceutical compositions containing them.

According to one aspect of the specification there is provided a compound of Formula (I):

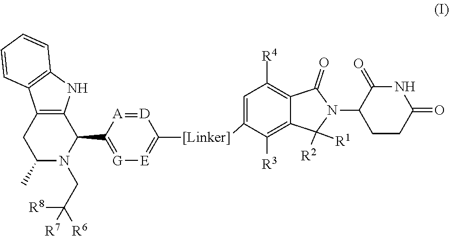

or a pharmaceutically acceptable salt thereof, wherein:
A and G are independently CR⁵ or N;
D and E are independently CH or N;

### R¹ is H;

### R² is H;

or R¹ and R² together with the carbon to which they are attached form carbonyl;

### R³ is H or OMe;

### R⁴ is H or OMe;

R⁵ is independently selected from H, F, Cl, CN, Me or OMe;

### R⁶ is H, Me or F;

### R⁷ is H, Me or F;

or R⁶ and R⁷ taken together with the carbon atom to which they are attached form a cyclopropyl ring or an oxetanyl ring;
R⁸ is H, Me, F, CH₂F, CHF₂, CF₃, CN, CH₂CN, CH₂OMe, CH₂OH, C(O)OH, C(O)OMe or SO₂Me;
Linker is an optionally substituted linking moiety comprising a branched or unbranched, cyclized or uncyclized, saturated or unsaturated chain of 6 to 15 carbon atoms in length, wherein 1 to 6 of the carbon atoms are optionally replaced with a heteroatom independently selected from O, N and S.

This specification also describes, in part, a pharmaceutical composition which comprises a compound of Formula (I), or a pharmaceutically acceptable salt thereof, and at least one pharmaceutically acceptable excipient.

This specification also describes, in part, a compound of Formula (I), or a pharmaceutically acceptable salt thereof, for use in therapy.

This specification also describes, in part, a compound of Formula (I), or a pharmaceutically acceptable salt thereof, for use in the treatment of cancer.

This specification also describes, in part, a method for treating cancer in a warm-blooded animal in need of such treatment, which comprises administering to the warm-blooded animal a therapeutically effective amount of a compound of Formula (I), or a pharmaceutically acceptable salt thereof.

### DESCRIPTION OF ILLUSTRATIVE EMBODIMENTS

Many embodiments of the disclosure are detailed throughout the specification and will be apparent to a reader skilled in the art. The disclosure is not to be interpreted as being limited to any particular embodiment(s) thereof.

In a first aspect there is provided a compound of Formula (I):

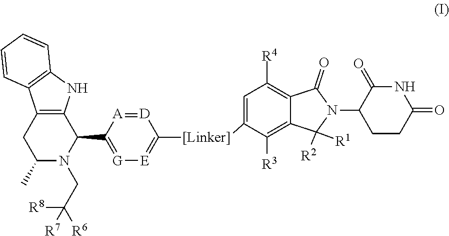

or a pharmaceutically acceptable salt thereof, wherein:
A and G are independently CR⁵ or N;
D and E are independently CH or N;

### R¹ is H;

### R² is H;

or R¹ and R² together with the carbon to which they are attached form carbonyl;

### R³ is H or OMe;

### R⁴ is H or OMe;

R⁵ is independently selected from H, F, Cl, CN, Me or OMe;

### R⁶ is H, Me or F;

### R⁷ is H, Me or F;

or R⁶ and R⁷ taken together with the carbon atom to which they are attached form a cyclopropyl ring or an oxetanyl ring;
R⁸ is H, Me, F, CH₂F, CHF₂, CF₃, CN, CH₂CN, CH₂OMe, CH₂OH, C(O)OH, C(O)OMe or SO₂Me;
Linker is an optionally substituted linking moiety comprising a branched or unbranched, cyclized or uncyclized, saturated or unsaturated chain of 6 to 15 carbon atoms in length, wherein 1 to 6 of the carbon atoms are optionally replaced with a heteroatom independently selected from O, N and S.

When the Linker comprises a cyclized chain, i.e. the Linker contains a ring, the length of the Linker chain is calculated based on the shortest route around the ring. For example, if the Linker contains the group

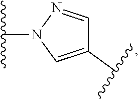 [SMILES: *c1cnn(*)c1]

this group contributes 3 atoms to the chain length as this is the shortest route around the ring.

As used herein the term “alkyl” refers to both straight and branched chain saturated hydrocarbon radicals having the specified number of carbon atoms.

As used herein the term “alkylene” refers to both straight and branched chain saturated divalent hydrocarbon radicals having the specified number of carbon atoms. Examples of alkylene include methylene, ethylene, propylene, butylene, pentylene and hexylene.

In certain embodiments, one to four units of —CH₂— in the alkylene chain may optionally be independently replaced with —O—, —NH—, —NMe-, cycloalkyl, heterocycloalkyl, aryl or heteroaryl. In such embodiments, it will be appreciated that the alkylene chain does not contain an acetal, peroxide, aminoacetal or azo group, for example, there are at least two methylene groups between each oxygen and/or nitrogen atom.

As used herein the term “branched” means that the total number of carbon atoms in the branch is no more than 4. Examples of a branched alkylene include —C₂H₄C(CH₃)₂C₂H₄OCH₂— which has two carbon atoms in the branch, and —CH(CH₃)—, which has one carbon atom in the branch.

In this specification the prefix C_{x-y}, as used in terms such as “C_{x-y} alkylene” and the like where x and y are integers, indicates the numerical range of carbon atoms that are present in the group. Examples of suitable C₁₋₃ alkylene groups, for example, include methylene, ethylene and propylene.

As used herein the term “cycloalkyl” refers to a non-aromatic, monocyclic or bicyclic carbocyclic ring. The term “C₄₋₁₀ cycloalkyl” refers to any such cycloalkyl group comprising 4 to 10 carbon atoms. In one embodiment, the cycloalkyl is a bicyclic carbocyclic ring. The term “C₃₋₆cycloalkyl” refers to any such cycloalkyl group comprising 3 to 6 carbon atoms. In one embodiment, the cycloalkyl is a monocyclic carbocyclic ring. Examples of suitable cycloalkyl groups include cyclobutyl.

As used herein, unless specified otherwise, the term “heterocycloalkyl” refers to a non-aromatic, monocyclic or bicyclic ring comprising one, two or three heteroatoms, for example one or two heteroatoms, selected from N, O or S; or an N-oxide thereof, or an S-oxide or S-dioxide thereof. The term “monocyclic heterocycloalkyl” refers to a monocyclic heterocycloalkyl group containing 3 to 5 carbon atoms and one or two heteroatoms independently selected from N, O or S; or an N-oxide thereof, or an S-oxide or S-dioxide thereof. Examples of suitable monocyclic heterocycloalkyl groups include azetidinyl, piperidinyl and piperazinyl. The term “bicyclic heterocycloalkyl” as used herein refers to a bicyclic heterocycloalkyl group containing 5 to 9 carbon atoms and one, two or three heteroatoms independently selected from N, O or S, for example, one or two heteroatoms independently selected from N, O or S; or an N-oxide thereof, or an S-oxide or S-dioxide thereof. The bicyclic heterocycloalkyl may be spirocyclic, fused or bridged. In one embodiment, the bicyclic heterocycloalkyl is spirocyclic. For the avoidance of doubt, substituents on the heterocycloalkyl group may be linked via either a carbon atom or a heteroatom. Examples of suitable bicyclic heterocycloalkyl groups include 3,9-diazaspiro[5.5]undecan-3-yl, 7-oxa-3,10-diazaspiro[5.6]dodecan-3-yl, 3-oxopiperazin-1-yl, 2,7-diazaspiro[3.5]nonan-7-yl, 2,6-diazaspiro[3.3]heptan-2-yl, 2,5-diazabicyclo[2.2.1]heptan-2-yl, 7-oxa-3,10-diazaspiro[5.6]dodecan-10-yl, 7-azaspiro[3.5]nonan-2-yl, 2-oxo-3,9-diazaspiro[5.5]undecan-3-yl, 2,7-diazaspiro[3.5]nonan-2-yl, 6-azaspiro[2.5]octan-1-yl and 3-azaspiro[5.5]undecan-3-yl. Any heterocycloalkyl optionally bears 1 or 2 oxo substituents. Examples of such heterocycloalkyls include 2-oxo-3,9-diazaspiro[5.5]undecan-3-yl and 3-oxopiperazin-1-yl.

As used herein the term “aryl” refers to a 6-membered monocyclic aromatic ring containing no heteroatoms. Aryl includes phenyl.

As used herein the term “heteroaryl” refers to a monocyclic or bicyclic heteroaryl. The term “monocyclic heteroaryl” as used herein refers to a 5- or 6-membered aromatic monocyclic ring system containing at least one heteroatom selected from O, S or N and includes 6-membered rings in which an aromatic tautomer exists. The term “bicyclic heteroaryl” as used herein refers to a bicyclic group comprising a first aromatic ring fused to a second aromatic ring to form a 6,5- or a 6,6-ring system, wherein at least one of the rings in the bicyclic group contains at least one heteroatom selected from O, S or N.

For the further avoidance of doubt, the use of “ [symbol] ” or “ [symbol] ” in formulae of this specification denotes the point of attachment between different groups.

The portion of Formula (I) represented as:

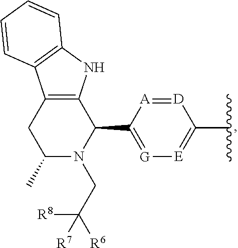

i.e. to the left-hand side of Linker may also be referred to herein as “ER binder”.

The portion of Formula (I) represented as:

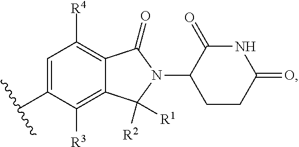

i.e. to the right-hand side of Linker may also be referred to herein as the “E3 ligase warhead”.

Where the term “optionally” is used, it is intended that the subsequent feature may or may not occur. As such, use of the term “optionally” includes instances where the feature is present, and also instances where the feature is not present. For example, a group “optionally substituted by F” includes groups with and without an F substituent.

The term “substituted” means that one or more hydrogens (for example one or two hydrogens, or alternatively one hydrogen) on the designated group is replaced by the indicated substituent(s) (for example one or two substituents, or alternatively one substituent), provided that any atom(s) bearing a substituent maintains a permitted valency. Substituent combinations encompass only stable compounds and stable synthetic intermediates. “Stable” means that the relevant compound or intermediate is sufficiently robust to be isolated and have utility either as a synthetic intermediate or as an agent having potential therapeutic utility. If a group is not described as “substituted”, or “optionally substituted”, it is to be regarded as unsubstituted (i.e. that none of the hydrogens on the designated group have been replaced).

The term “pharmaceutically acceptable” is used to specify that an object (for example a salt, dosage form or excipient) is suitable for use in patients. An example list of pharmaceutically acceptable salts can be found in the Handbook of Pharmaceutical Salts: Properties, Selection and Use, P. H. Stahl and C. G. Wermuth, editors, Weinheim/Zürich: Wiley-VCH/VHCA, 2002.

A suitable pharmaceutically acceptable salt of a compound of the Formula (I) is, for example, a salt formed within the human or animal body after administration of a compound of the Formula (I), to said human or animal body.

A further embodiment provides any of the embodiments defined herein (for example the embodiment of claim 1) with the proviso that one or more specific Examples (for instance one, two or three specific Examples) selected from the group consisting of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and 41 is individually disclaimed.

A further embodiment provides any of the embodiments defined herein (for example the embodiment of claim 1) with the proviso that one or more specific Examples (for instance one, two or three specific Examples) selected from the group consisting of Examples 1, 2, 3, 4 and 5 is individually disclaimed.

Some values of variable groups in Formula (I) are as follows.

In one embodiment, A is CR⁵.

In one embodiment, G is CR⁵.

In one embodiment, A is CR⁵ and G is CR⁵.

In one embodiment, A is CR⁵ and G is N.

In one embodiment, A is N and G is CR⁵.

In one embodiment, R⁵ is independently selected from H, F, Cl, CN, Me or OMe.

In one embodiment, R⁵ is independently H or F.

In one embodiment, R⁵ is H.

In one embodiment, R⁵ is F.

In one embodiment, A is CR⁵ and R⁵ is H, F, Cl, CN, Me or OMe.

In one embodiment, A is CR⁵ and R⁵ is H or F.

In one embodiment, G is CR⁵ and R⁵ is H, F, Cl, CN, Me or OMe.

In one embodiment, G is CR⁵ and R⁵ is H or F.

In one embodiment, G is N.

In one embodiment, A is CH and G is CH.

In one embodiment, A is CF and G is CF.

In one embodiment, A is N and G is CF.

In one embodiment, A is N and G is CH.

In one embodiment, A is CF and G is N.

In one embodiment, A is CH and G is N.

In one embodiment, D is CH.

In one embodiment, E is CH.

In one embodiment, both D and E are CH.

In one embodiment, both D and E are N.

In one embodiment, A and G are both CF and D and E are both CH, or A and G are both CH and D and E are both N, or A is CH and G is N and D and E are both CH.

In one embodiment, A and G are both CF and D and E are both CH, or A and G are both CH and D and E are both N.

In one embodiment, the moiety:

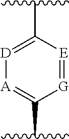

is selected from the group consisting of

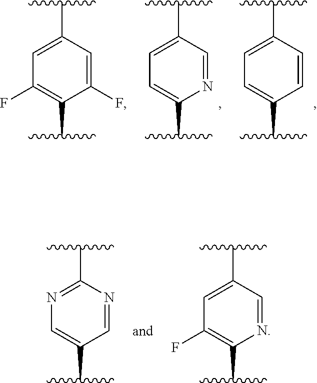

In one embodiment, the moiety:

is selected from the group consisting of

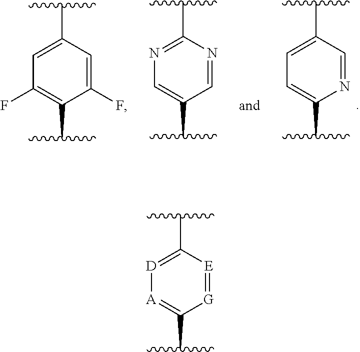

In one embodiment, the moiety:

is selected from the group consisting of

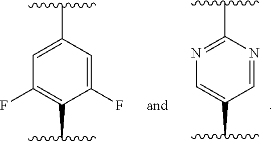

In one embodiment, R¹ is H.

In one embodiment R² is H.

In another embodiment, R¹ and R² together with the carbon to which they are attached form carbonyl.

In one embodiment, R³ is H.

In another embodiment R³ is OMe.

In one embodiment, R⁴ is H.

In another embodiment R⁴ is OMe.

In one embodiment, one of R³ or R⁴ is OMe and the other is H.

In one embodiment, R⁴ is OMe and R³ is H.

In one embodiment, R⁶ is H. In one embodiment, R⁶ is Me. In another embodiment, R⁶ is F.

In one embodiment, R⁷ is H. In one embodiment, R⁷ is Me. In another embodiment, R⁷ is F.

In one embodiment, R⁶ and R⁷ taken together with the carbon atom to which they are attached form a cyclopropyl or an oxetane ring.

In one embodiment, R⁶ and R⁷ taken together with the carbon atom to which they are attached form a cyclopropyl ring.

In one embodiment, R⁶ and R⁷ taken together with the carbon atom to which they are attached form an oxetane ring.

In one embodiment, R⁸ represents H, Me, F, CH₂F, CHF₂, CF₃, CN, CH₂CN, CH₂OMe, CH₂OH, C(O)OH, C(O)OMe or SO₂Me. In one embodiment, R⁸ is selected from H, Me, F, C(O)OH and C(O)OMe. In one embodiment, R⁸ is H. In another embodiment, R⁸ is Me. In another embodiment, R⁸ is F. In another embodiment, R⁸ is CH₂F. In another embodiment, R⁸ is CHF₂. In another embodiment, R⁸ is CF₃. In another embodiment, R⁸ is CN. In another embodiment, R⁸ is CH₂CN. In another embodiment, R⁸ is CH₂OMe. In another embodiment, R¹ is CH₂OH. In another embodiment, R¹ is C(O)OH. In another embodiment, R¹ is C(O)OMe. In another embodiment, R⁸ is SO₂Me.

In one embodiment, R⁶, R⁷ and R⁸ each represent F. In another embodiment, R⁶ and R⁷ each represent H and R⁸ represents F.

In one embodiment, the group —CH₂—C(R⁶)(R⁷)(R⁸) is selected from the group consisting of:

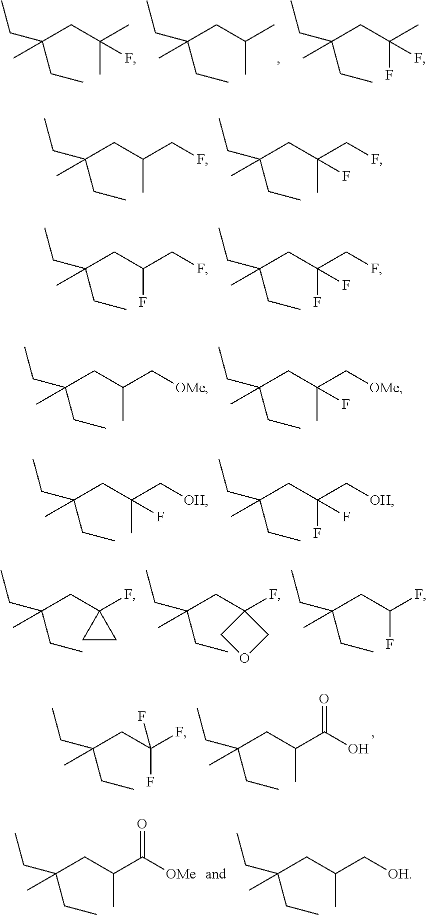

In one embodiment, the group —CH₂—C(R⁶)(R⁷)(R⁸) is selected from the group consisting of:

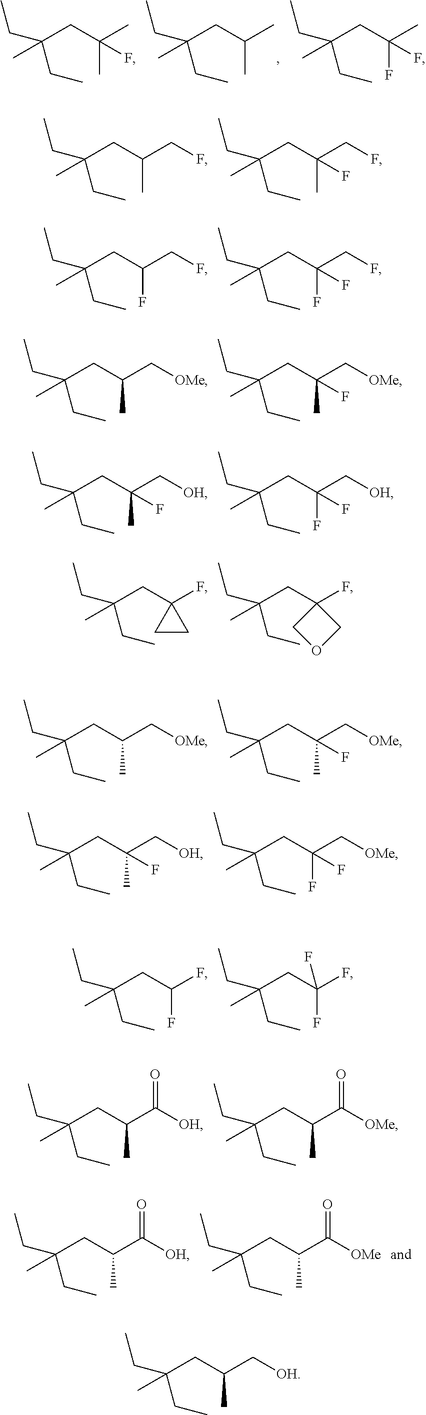

In one embodiment, the group —CH₂—C(R⁶)(R⁷)(R⁸) is selected from the group consisting of:

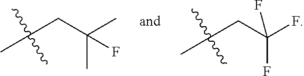

In one embodiment, the Linker is an optionally substituted linking moiety comprising a branched or unbranched, cyclized or uncyclized, saturated or unsaturated chain of 6 to 15 carbon atoms in length, wherein 1 to 4 of the carbon atoms are optionally replaced with a heteroatom independently selected from O and N.

In one embodiment, the Linker is an optionally substituted linking moiety comprising a branched or unbranced, cyclized or uncyclized, saturated or unsaturated chain of 6 to 12 carbon atoms in length, wherein 1 to 4 of the carbon atoms are optionally replaced with a heteroatom independently selected from O, N and S.

In one embodiment, the Linker is an optionally substituted linking moiety comprising a branched or unbranced, cyclized or uncyclized, saturated or unsaturated chain of 6 to 12 carbon atoms in length, wherein 1 to 4 of the carbon atoms are optionally replaced with a heteroatom independently selected from O and N.

In one embodiment, the Linker is optionally substituted with oxo to form a carbonyl group within the Linker, i.e. two hydrogens of a carbon atom in the Linker are replaced by a single oxo (═O).

In one embodiment, the chain of the Linker is an unbranched, cyclized, saturated chain.

In one embodiment, the Linker is a C₃₋₁₄ alkylene chain wherein one to four —CH₂— units in the alkylene chain are independently optionally replaced with a group independently selected from —C(O)—, —O—, —NH—, —NMe-, cycloalkyl, heterocycloalkyl, aryl and heteroaryl.

In one embodiment, the Linker is a C₃₋₁₄ alkylene chain wherein one to four —CH₂— units in the alkylene chain are independently optionally replaced with a group independently selected from —O—, —NH—, —NMe-, cycloalkyl, heterocycloalkyl, aryl and heteroaryl.

In one embodiment, one to four —CH₂— units in the alkylene chain are optionally replaced with a group independently selected from —O—, —NMe-, cycloalkyl and heterocycloalkyl.

In one embodiment, one to four —CH₂— units in the C₃₋₁₄ alkylene chain are independently optionally replaced with a group selected from —O—, cycloalkyl and heterocycloalkyl.

In one embodiment, one to four —CH₂— units in the C₃₋₁₄ alkylene chain are independently optionally replaced with a group selected from —O— and heterocycloalkyl.

In one embodiment, one to four —CH₂— units in the C₃₋₁₄alkylene chain are independently optionally replaced with a group selected from —O—, —NMe-, cycloalkyl and a nitrogen containing heterocycloalkyl group.

Any heterocycloalkyl optionally bears 1 or 2, for example 1, oxo substituent(s).

In one embodiment, the Linker is a C₃-alkylene chain.

In one embodiment, the Linker is an unbranched alkylene chain.

In one embodiment, the Linker is a branched alkylene chain.

In another embodiment, the Linker is an unbranched C₃₋₇alkylene chain.

In another embodiment, the Linker is a branched C₃₋₇alkylene chain.

In one embodiment, one to four —CH₂— units in the C₃₋₁₄ alkylene chain are independently optionally replaced with a group selected from —O— and a nitrogen containing heterocycloalkyl group.

In one embodiment, no more than three —CH₂— units are replaced with a nitrogen containing heterocycloalkyl group.

In one embodiment Linker is represented by the moiety —X—[W]ₚ-Het¹-, wherein:

X is selected from the group consisting of -Het²-C₁₋₆alkylene, —C(O)—Het²-C₁₋₆alkylene, -Het²-C(O)—C₁₋₆alkylene, —C₁₋₆alkenylene, —O-Het²-C₁₋₆alkylene, —C₁₋₆alkylene- and —O-Cyc-C₁₋₆alkylene, wherein one or two —CH₂— units in the alkylene chain is independently replaced with —O—, —NH— or —NMe-;

W is selected from -Het³-C₁₋₆ alkylene;

Het¹ is a nitrogen containing monocyclic or bicyclic heterocycloalkyl group;

Het² is a nitrogen containing monocyclic or bicyclic heterocycloalkyl group;

Het³ is a nitrogen containing monocyclic or bicyclic heterocycloalkyl group;

Cyc is C₃₋₆cycloalkyl;

p is 0 or 1;

wherein heterocycloalkyl is optionally substituted with 1 or 2 oxo substituents.

The Het¹ portion of the Linker is directly attached to the E3 ligase warhead and the X portion of the Linker is directly attached to the ER binder. When p is 0, the alkylene group within the X portion of the Linker is directly attached to Het¹ and when p is 1, the alkylene group within the X portion of the Linker is directly attached to W.

In one embodiment, the E3 ligase warhead is attached via a nitrogen atom in Het¹.

In one embodiment, X is selected from -Het²-C₁₋₆ alkylene, —C(O)—Het²-C₁₋₆ alkylene, —C₁₋₆ alkylene, —O-Het²-C₁₋₆alkylene and —O-Cyc-C₁₋₆alkylene.

In one embodiment, X is selected from -Het²-C₁₋₆ alkylene, —C₁₋₆ alkylene, —O-Het²-C₁₋₆alkylene and —O-Cyc-C₁₋₆alkylene.

In one embodiment, X is selected from -Het²-C₁₋₆ alkylene, —C₁₋₆ alkylene, —O-Het²-C₁₋₃alkylene and —O-Cyc-C₁₋₃alkylene.

In one embodiment, X is selected from -Het²-methylene, X is -Het²-ethylene, -Het²-propylene, hexylene, —O-pentylene, —C(O)—Het²-methylene, -Het²-O-ethylene, -Het²-O-ethylene, -Het²-CH₂N(Me)-, -Het²-(CH₂)₂N(Me)-, -Het²-CH(Me)-, —O-Cyc-ethylene, —O-Cyc-ethylene and —O-Het²-methylene.

In one embodiment, X is selected from -Het²-methylene, -Het²-ethylene, -Het²-propylene, -Het²-O-ethylene, -Het²-O-propylene, —O-pentylene, -Het²-CH₂N(Me)-, —O-Cyc-ethylene, -Het²-(CH₂)₂N(Me)-, -Het²-CH(Me)- and —O-Het²-methylene.

In one embodiment, X is -Het²-methylene.

In one embodiment, X is -Het²-ethylene.

In one embodiment, X is -Het²-propylene.

In one embodiment, X is hexylene.

In one embodiment, X is —O-pentylene.

In one embodiment, X is —C(O)—Het²-methylene.

In one embodiment, X is -Het²-O-ethylene.

In one embodiment, X is -Het²-O-propylene.

In one embodiment, X is -Het²-CH₂N(Me)-.

In one embodiment, X is -Het²-(CH₂)₂N(Me)-.

In one embodiment, X is -Het²-CH(Me)-.

In one embodiment, X is —O-Cyc-ethylene.

In one embodiment, X is —O-Het²-methylene.

In one embodiment, p is 0.

In one embodiment, p is 1.

When p is 1, W is present and when p is 0, W is absent.

In one embodiment, W is selected from -Het³-C₁₋₃alkylene.

In one embodiment, -Het³-methylene.

In one embodiment, Het¹ is selected from the group consisting of piperazinyl, piperidinyl, azetidinyl, a nitrogen containing spirobicyclic heterocycloalkyl and a nitrogen containing bridged bicyclic heterocycloalkyl.

In one embodiment, Het¹ is selected from the group consisting of piperidin-1-yl, piperazin-1-yl, 3,9-diazaspiro[5.5]undecan-3-yl, 7-oxa-3,10-diazaspiro[5.6]dodecan-3-yl, 3-oxopiperazin-1-yl, 2,7-diazaspiro[3.5]nonan-7-yl, 2,6-diazaspiro[3.3]heptan-2-yl, azetidin-1-yl and 2,5-diazabicyclo[2.2.1]heptan-2-yl.

In one embodiment, Het¹ is piperidin-1-yl.

In one embodiment, Het¹ is piperazin-1-yl.

In one embodiment, Het¹ is 3,9-diazaspiro[5.5]undecan-3-yl.

In one embodiment, Het¹ is 7-oxa-3,10-diazaspiro[5.6]dodecan-3-yl.

In one embodiment, Het¹ is 3-oxopiperazin-1-yl.

In one embodiment, Het¹ is 2,7-diazaspiro[3.5]nonan-7-yl.

In one embodiment, Het¹ is 2,6-diazaspiro[3.3]heptan-2-yl.

In one embodiment, Het¹ is azetidin-1-yl.

In one embodiment, Het¹ is 2,5-diazabicyclo[2.2.1]heptan-2-yl.

In one embodiment, Het² is selected from the group consisting of piperidinyl, azetidinyl and a nitrogen containing spirobicyclic heterocycloalkyl.

In one embodiment, Het² is selected from the group consisting of piperidin-4-yl, 3,9-diazaspiro[5.5]undecan-3-yl, 7-oxa-3,10-diazaspiro[5.6]dodecan-10-yl, 7-azaspiro[3.5]nonan-2-yl, 2-oxo-3,9-diazaspiro[5.5]undecan-3-yl, 2,7-diazaspiro[3.5]nonan-2-yl, 6-azaspiro[2.5]octan-1-yl, azetidin-3-yl and 3-azaspiro[5.5]undecan-3-yl.

In one embodiment, Het² is piperidin-4-yl.

In one embodiment, Het² is 3,9-diazaspiro[5.5]undecan-3-yl.

In one embodiment, Het² is 7-oxa-3,10-diazaspiro[5.6]dodecan-10-yl.

In one embodiment, Het² is 7-azaspiro[3.5]nonan-2-yl.

In one embodiment, Het² is 2-oxo-3,9-diazaspiro[5.5]undecan-3-yl.

In one embodiment, Het² is 2,7-diazaspiro[3.5]nonan-2-yl.

In one embodiment, Het² is 6-azaspiro[2.5]octan-1-yl.

In one embodiment, Het² is azetidin-3-yl.

In one embodiment, Het² is 3-azaspiro[5.5]undecan-3-yl.

In one embodiment, Het³ is a nitrogen containing monocyclic heterocycloalkyl.

In one embodiment, Het³ is selected from the group consisting of piperidinyl, piperazinyl and azetidinyl.

In one embodiment, Het³ is selected from the group consisting of piperidin-4-yl, piperazin-1-yl and azetidin-1yl.

In one embodiment, Het³ is piperidinyl.

In one embodiment, Het³ is piperidin-4-yl.

In one embodiment, Het³ is piperazinyl.

In one embodiment, Het³ is piperazin-1-yl.

In one embodiment, Het³ is azetidinyl.

In one embodiment, Het³ is azetidin-1-yl.

In one embodiment, Cyc is cyclobutyl.

In one embodiment, X is selected from -Het²-C₁₋₆ alkylene, —C₁₋₆ alkylene, —O-Het²-C₁₋₆alkylene and —O-Cyc-C₁₋₆alkylene and Het² is selected from the group consisting of piperidinyl, azetidinyl and a nitrogen containing spirobicyclic heterocycloalkyl and Cyc is C₄₋₆cycloalkyl. In one embodiment, X is selected from Het²-C₁₋₆ alkylene, —C₁₋₆ alkylene, —O-Het²-C₁₋₃alkylene and —O-Cyc-C₁₋₃alkylene and Het² is selected from the group consisting of piperidin-1-yl, piperazin-1-yl, 3,9-diazaspiro[5.5]undecan-3-yl, 7-oxa-3,10-diazaspiro[5.6]dodecan-3-yl, 3-oxopiperazin-1-yl, 2,7-diazaspiro[3.5]nonan-7-yl, 2,6-diazaspiro[3.3]heptan-2-yl, azetidin-1-yl and 2,5-diazabicyclo[2.2.1]heptan-2-yl, and Cyc is cyclobutyl.

In one embodiment, W is -Het³-methylene and Het³ is a nitrogen containing monocyclic heterocycloalkyl.

In one embodiment, W is -Het³-methylene and Het³ is selected from the group consisting of piperidinyl, piperazinyl and azetidinyl.

In one embodiment Linker is represented by the moiety —X-Het¹-, wherein:

X is selected from -Het²-C₁₋₆ alkylene, —C(O)—Het²-C₁₋₆ alkylene, -Het²-C(O)—C₁₋₆ alkylene or —C₁₋₆ alkenylene, wherein one or two —CH₂— units in the alkylene chain is independently replaced with —O—, —NH— or —NMe-;

Het¹ is a nitrogen containing monocyclic or bicyclic heterocycloalkyl group; and

Het² is a nitrogen containing monocyclic or bicyclic heterocycloalkyl group.

The Het¹ portion of the Linker is directly attached to the E3 ligase warhead and the X portion of the Linker is directly attached to the ER binder. The alkylene group within the X portion of the Linker is directly attached to Het¹.

In one embodiment, the E3 ligase warhead is attached via a nitrogen atom in Het¹.

In one embodiment, X is selected from -Het²-C₁₋₆ alkylene-, —C(O)—Het²-C₁₋₆ alkylene- and —C₁₋₆ alkylene-.

In one embodiment, X is selected from -Het²-C₁₋₆ alkylene- and —C₁₋₆ alkylene-.

In one embodiment, X is -Het²-methylene-.

In one embodiment, X is -Het²-ethylene-.

In one embodiment, X is -Het²-propylene-.

In one embodiment, X is -hexylene-.

In one embodiment, X is —O-pentylene-.

In one embodiment, X is —C(O)—Het²-methylene-.

In one embodiment, X is -Het²-O-ethylene-.

In one embodiment, Het¹ is selected from the group consisting of piperazinyl, a nitrogen containing spirobicyclic heterocycloalkyl and a nitrogen containing bridged bicyclic heterocycloalkyl.

In one embodiment, Het¹ is selected from the group consisting of piperazin-1-yl, 2,6-diazaspiro[3,3]heptanyl, 1,2,3,3a,4,5,6,6a-octahydropyrrolo[3,4-c]pyrrole, 2,6-diazaspiro[3.3]heptane and 2,5-diazabicyclo[2.2.1]heptane.

In one embodiment, Het¹ is a monocyclic heterocycloalkyl group.

In one embodiment, Het¹ is piperazinyl.

In one embodiment, Het¹ is piperazin-1-yl.

In one embodiment, Het² is a monocyclic heterocycloalkyl group containing one ring nitrogen.

In one embodiment, Het² is selected from the group consisting of azetidinyl and piperidinyl.

In one embodiment, Het² is selected from group consisting of azetindin-1-yl and piperidin-1-yl.

In one embodiment, Het² is piperidinyl.

In one embodiment, Het² is piperidin-1-yl.

In one embodiment, X is selected from -Het²-C₁₋₆ alkylene- and —C₁₋₆ alkylene-; Het¹ is piperazinyl; and Het² is piperidinyl.

In one embodiment, Linker is selected from the group consisting of:

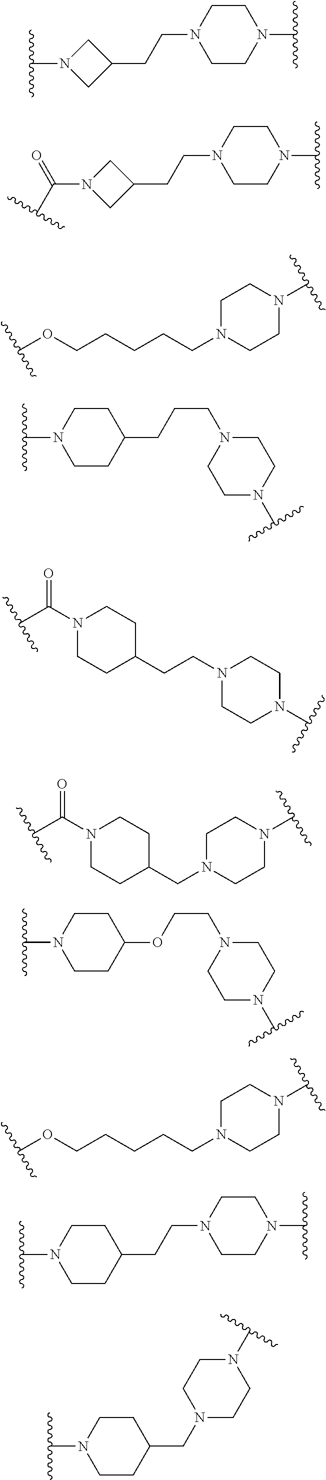

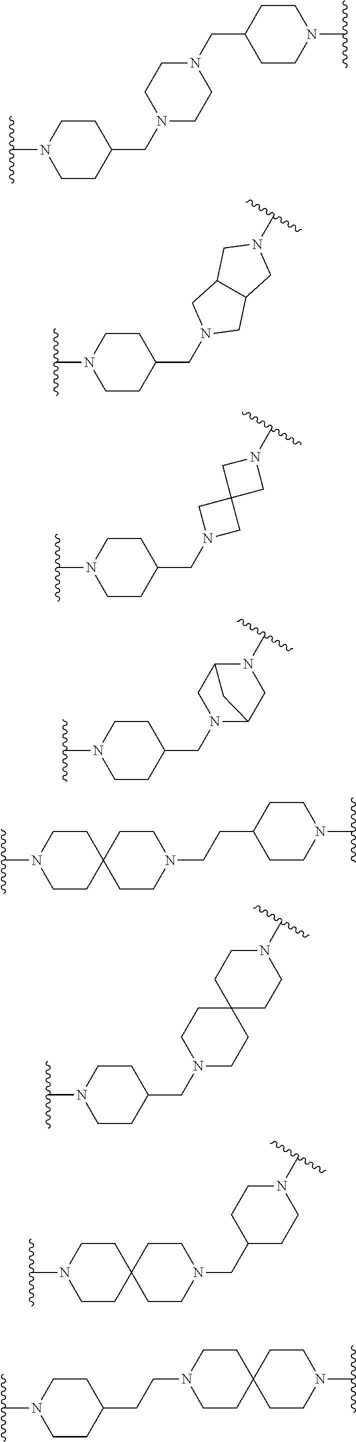

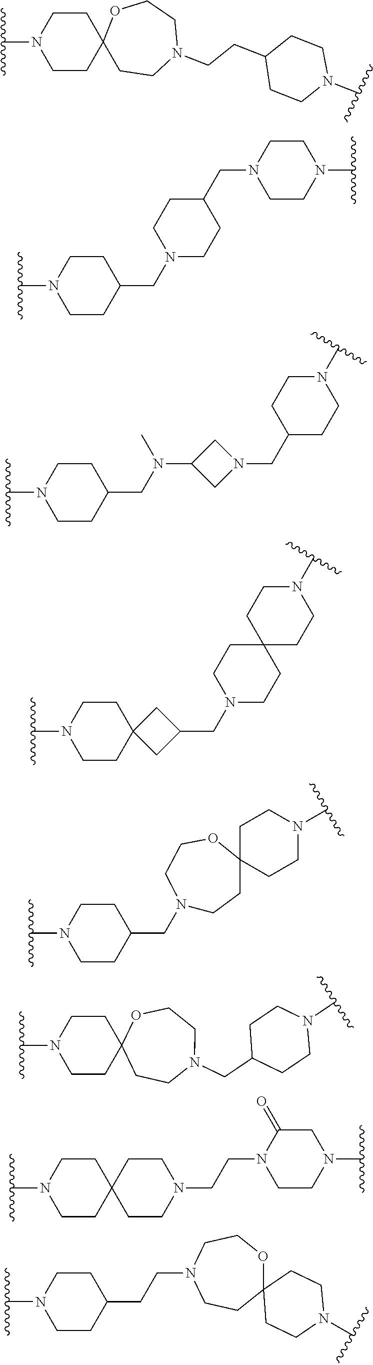

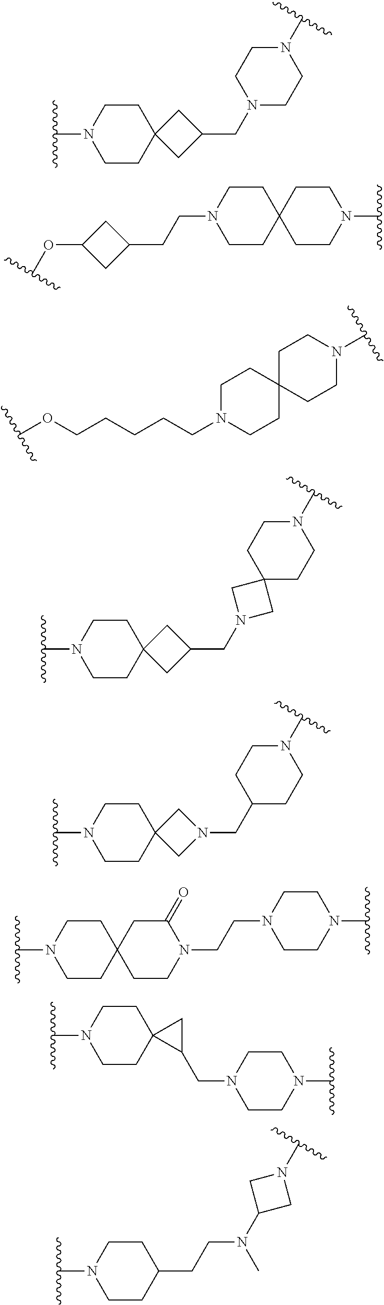

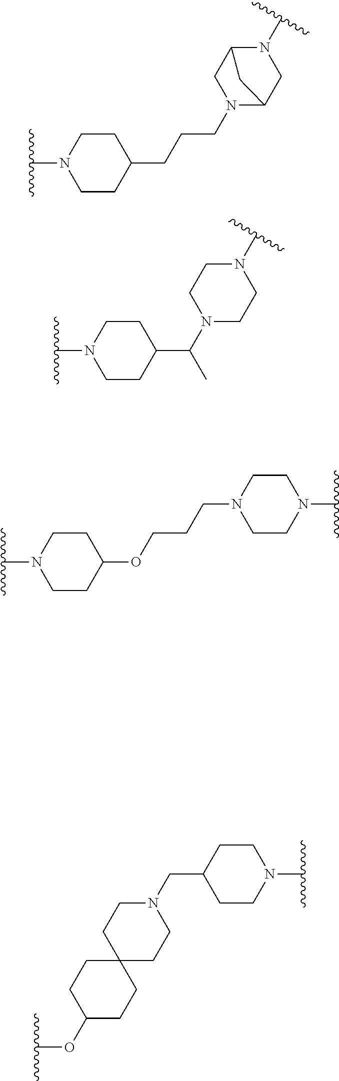 [SMILES: *N1CCC(CCCN2CC3CC2CN3*)CC1 ; *N1CCC(C(C)N2CCN(*)CC2)CC1 ; *N1CCC(OCCCN2CCN(*)CC2)CC1 ; *OC1CCC2(CC1)CCN(CC1CCN(*)CC1)CC2]

In one embodiment, Linker is selected from the group consisting of:

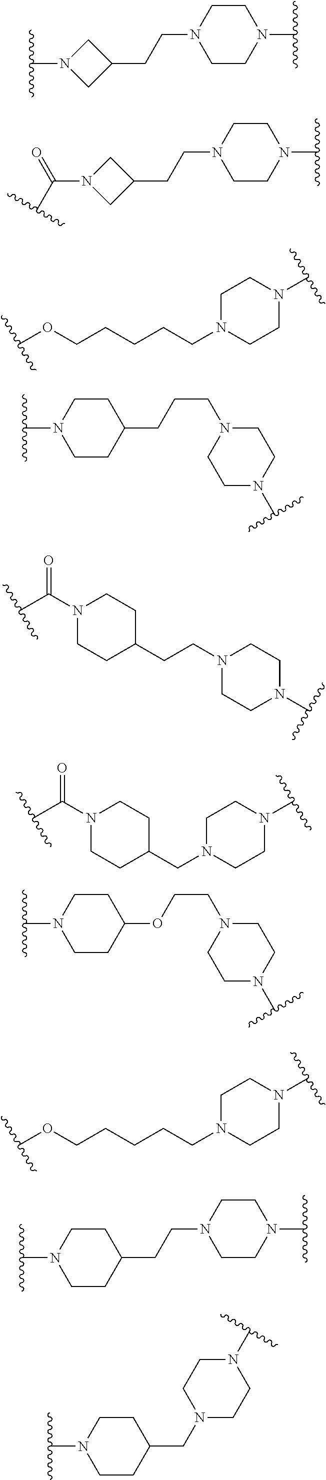

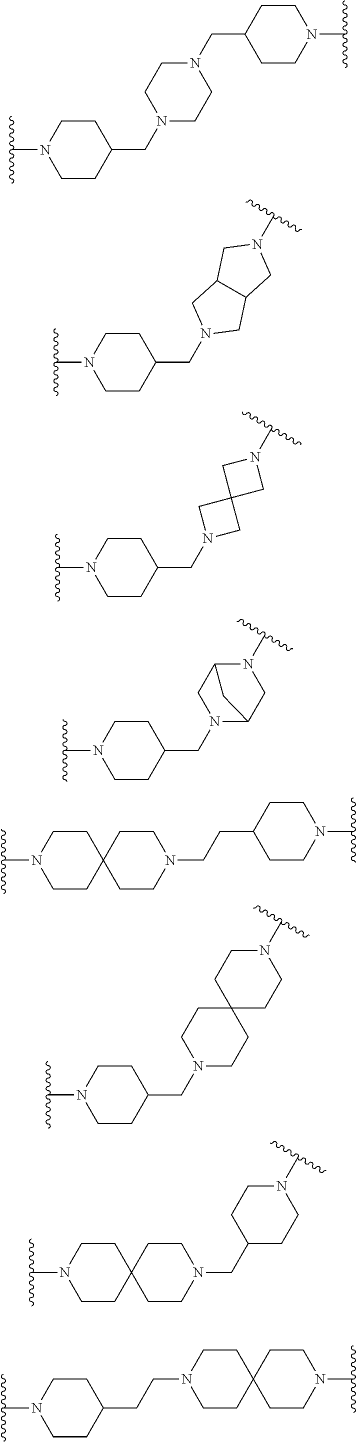

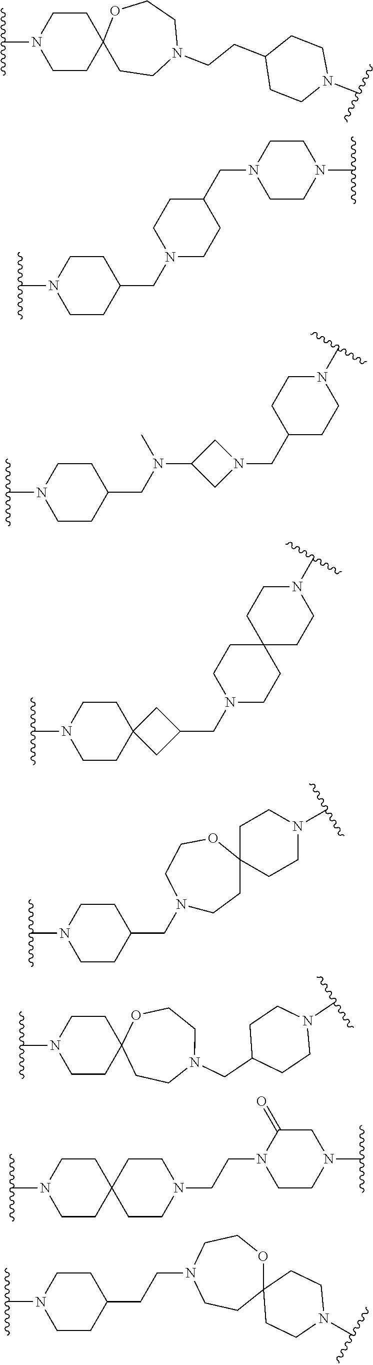

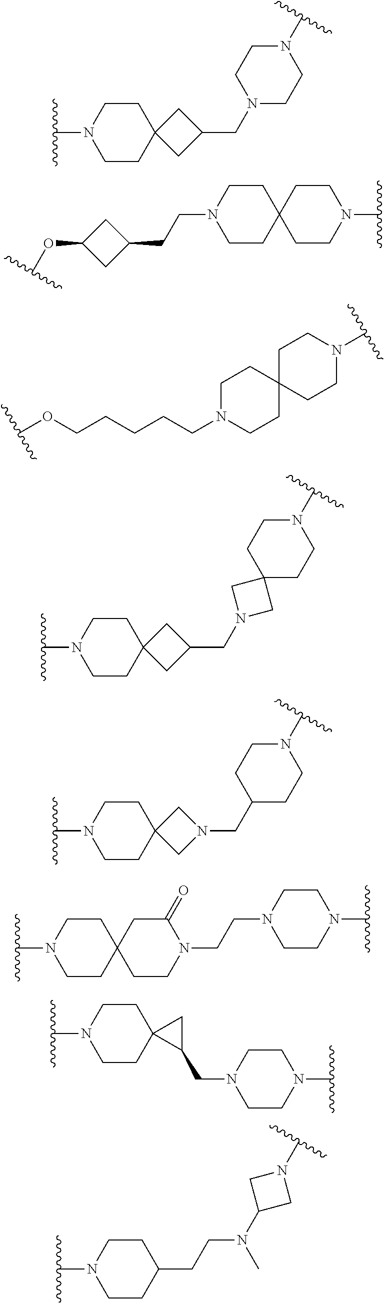

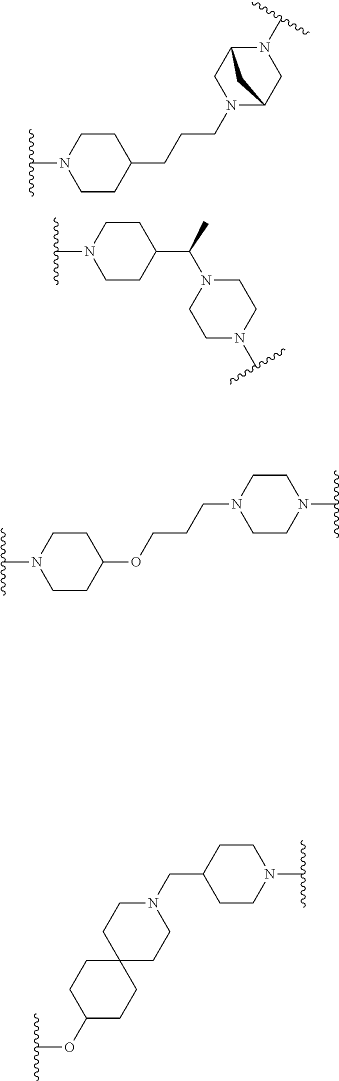

In one embodiment, Linker, or the moiety —X-Het¹-, is selected from the group consisting of:

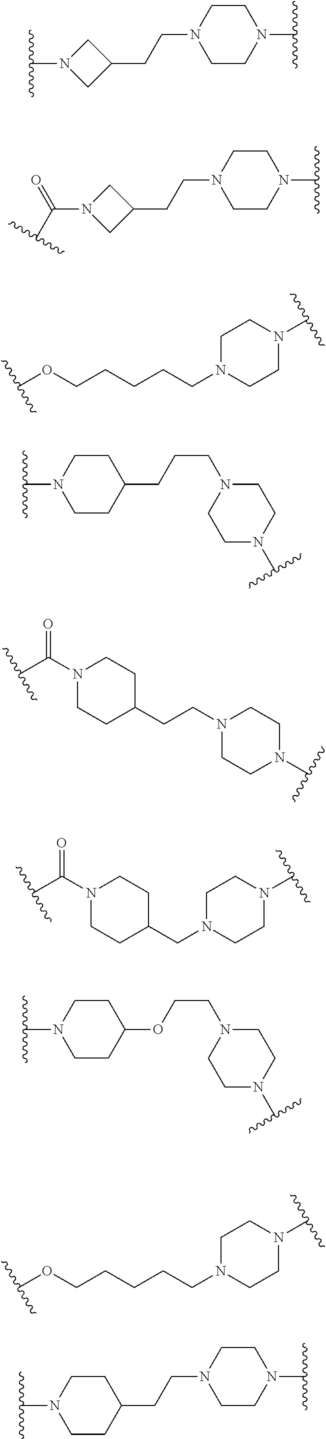

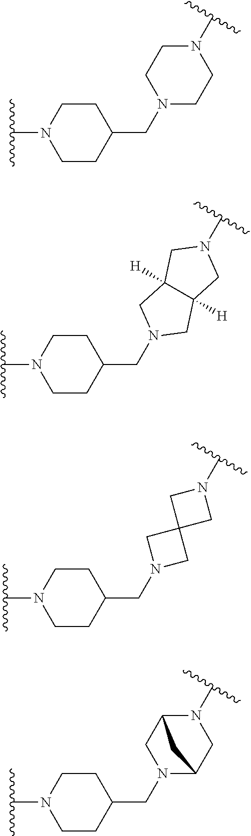 [SMILES: *N1CCC(CN2CCN(*)CC2)CC1 ; *N1CCC(CN2C[C@H]3CN(*)C[C@H]3C2)CC1 ; *N1CCC(CN2CC3(CN(*)C3)C2)CC1 ; *N1CCC(CN2C[C@H]3C[C@@H]2CN3*)CC1]

In one embodiment, Linker, or the moiety —X-Het¹-, is selected from the group consisting of:

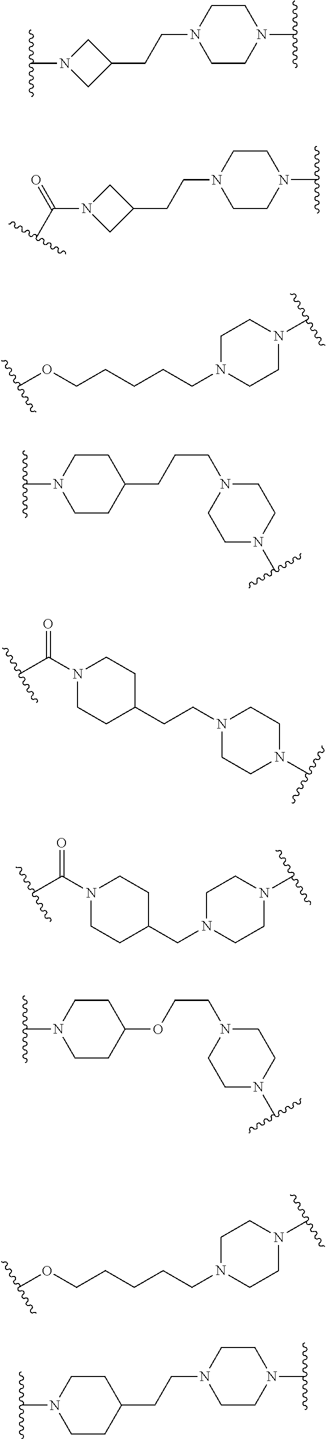

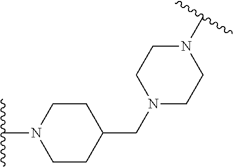 [SMILES: *N1CCC(CN2CCN(*)CC2)CC1]

In one embodiment, Linker is selected from the group consisting of:

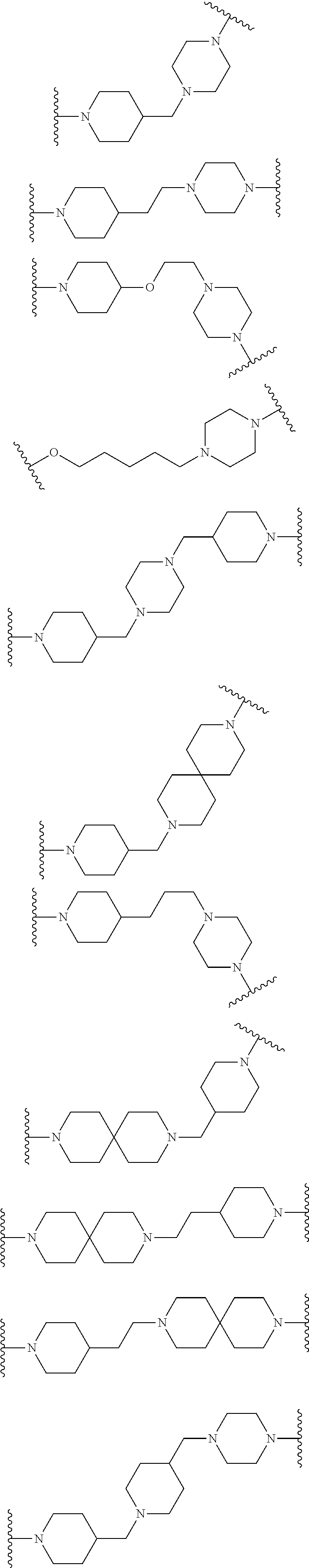

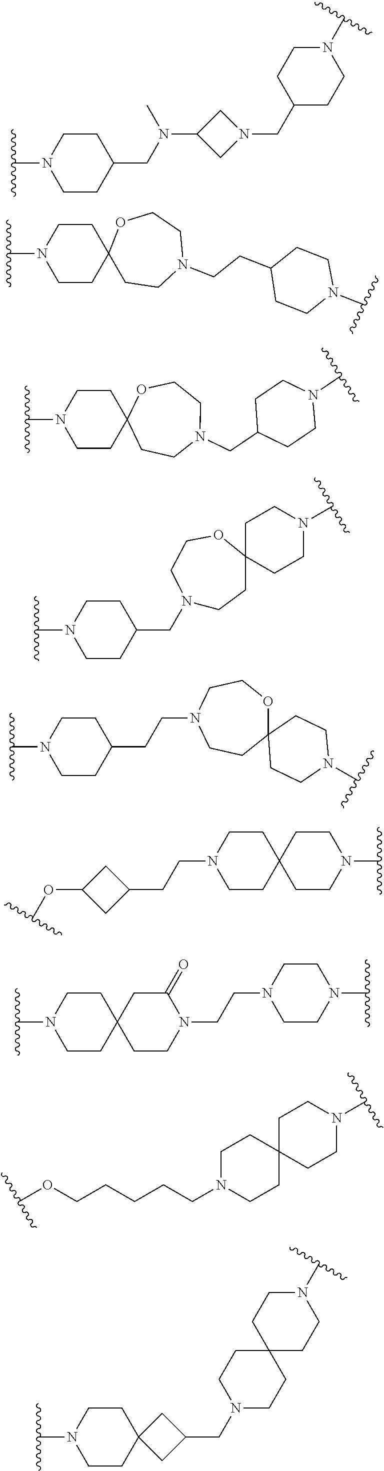

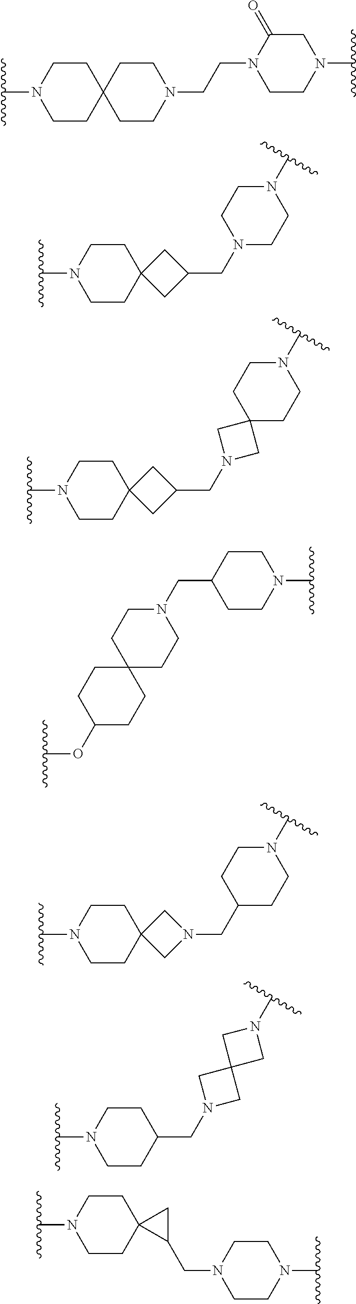

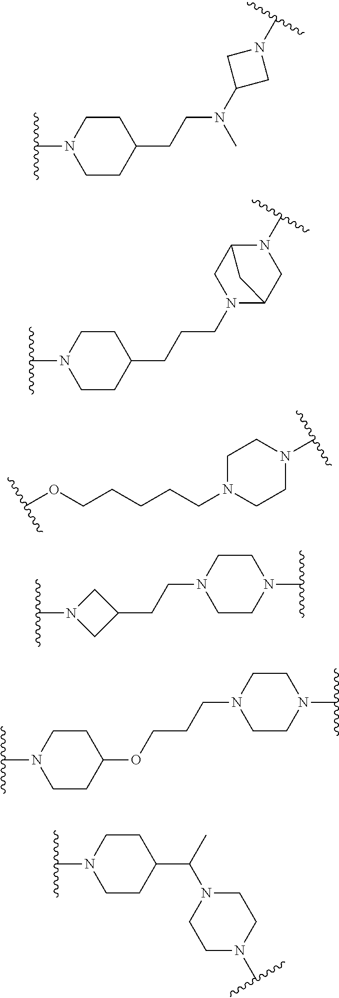 [SMILES: *N1CCC(CCN(C)C2CN(*)C2)CC1 ; *N1CCC(CCCN2CC3CC2CN3*)CC1 ; *OCCCCCN1CCN(*)CC1 ; *N1CCN(CCC2CN(*)C2)CC1 ; *N1CCC(OCCCN2CCN(*)CC2)CC1 ; *N1CCC(C(C)N2CCN(*)CC2)CC1]

In one embodiment, Linker is selected from the group consisting of:

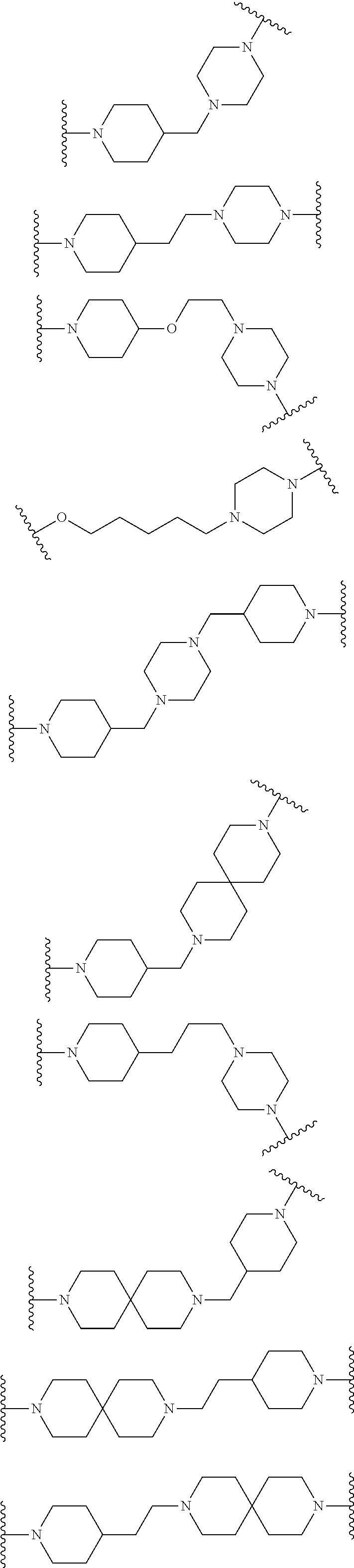

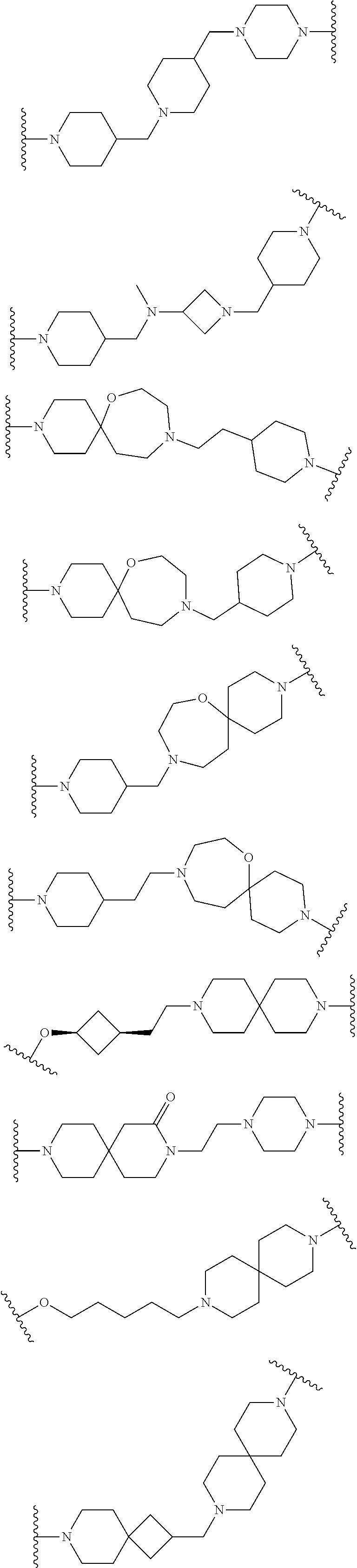

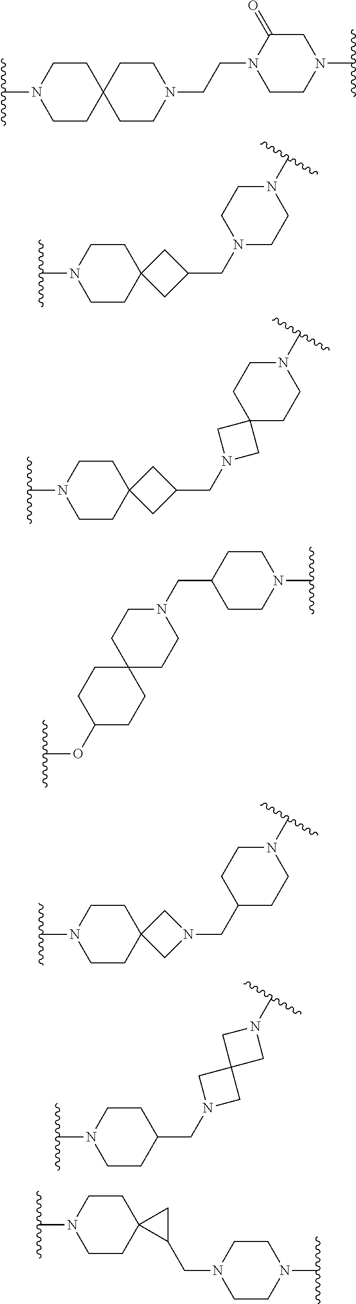

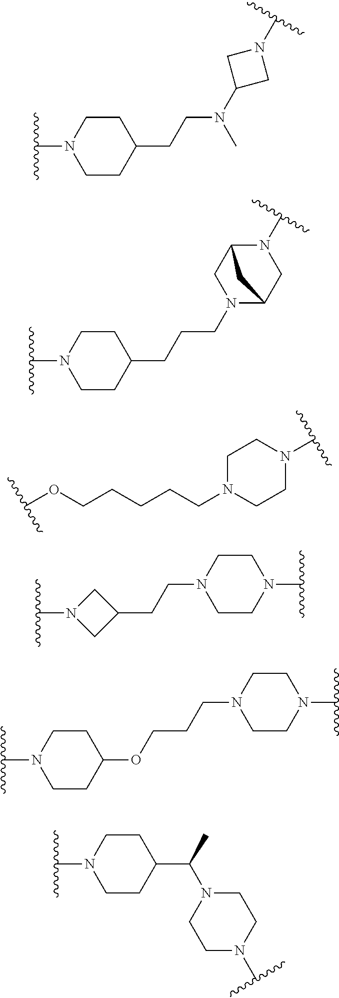 [SMILES: *N1CCC(CCN(C)C2CN(*)C2)CC1 ; *N1CCC(CCCN2C[C@H]3C[C@@H]2CN3*)CC1 ; *OCCCCCN1CCN(*)CC1 ; *N1CCN(CCC2CN(*)C2)CC1 ; *N1CCC(OCCCN2CCN(*)CC2)CC1 ; *N1CCC([C@@H](C)N2CCN(*)CC2)CC1]

In one embodiment, Linker, or the moiety —X-Het¹, is selected from the group consisting of:

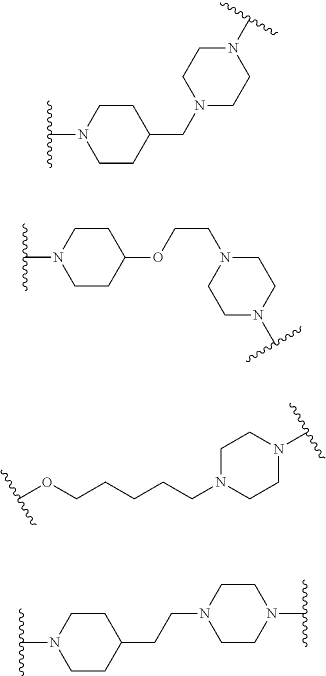 [SMILES: *N1CCC(CN2CCN(*)CC2)CC1 ; *N1CCC(OCCN2CCN(*)CC2)CC1 ; *OCCCCCN1CCN(*)CC1 ; *N1CCC(CCN2CCN(*)CC2)CC1]

In one embodiment, there is provided a compound of Formula (I), or a pharmaceutically acceptable salt thereof, wherein:

A and G are independently CR⁵ or N;

D and E are independently CH or N;

R¹ is H;

R² is H;

or R¹ and R² together with the carbon to which they are attached form carbonyl;

R³ is H or OMe;

R⁴ is H or OMe;

R⁵ is independently selected from H, F, Cl, CN, Me or OMe;

R⁶ is H, Me or F;

R⁷ is H, Me or F;

or R⁶ and R⁷ taken together with the carbon atom to which they are attached form a cyclopropyl ring or an oxetanyl ring;

R⁸ is H, Me, F, CH₂F, CHF₂, CF₃, CN, CH₂CN, CH₂OMe, CH₂OH, C(O)OH, C(O)OMe or SO₂Me;

Linker is represented by —X-Het¹-, wherein X is selected from -Het²-C₁₋₆ alkylene, —C(O)—Het²-C₁₋₆ alkylene, -Het²-C(O)—C₁₋₆ alkylene, —C₁₋₆ alkylene, wherein one or two —CH₂— units in the alkylene chain is replaced with —O—, —NH— or —NMe-; Het¹ is a nitrogen containing monocyclic or bicyclic heterocycloalkyl group; and Het² is a nitrogen containing monocyclic or bicyclic heterocycloalkyl group.

In one embodiment, Het¹ is selected from the group consisting of piperazin-1-yl, 2,6-diazaspiro[3,3]heptanyl, 1,2,3,3a,4,5,6,6a-octahydropyrrolo[3,4-c]pyrrole, 2,6-diazaspiro[3.3]heptane and 2,5-diazabicyclo[2.2.1]heptane.

In one embodiment, Het² is selected from group consisting of azetidinyl and piperidinyl.

In one embodiment, Het¹ is piperazinyl and X is selected from -Het²-C₁₋₆ alkylene and —C₁₋₆ alkylene, wherein Het² is piperidinyl.

In one embodiment, Linker, or —X-Het¹-, is selected from the group consisting of:

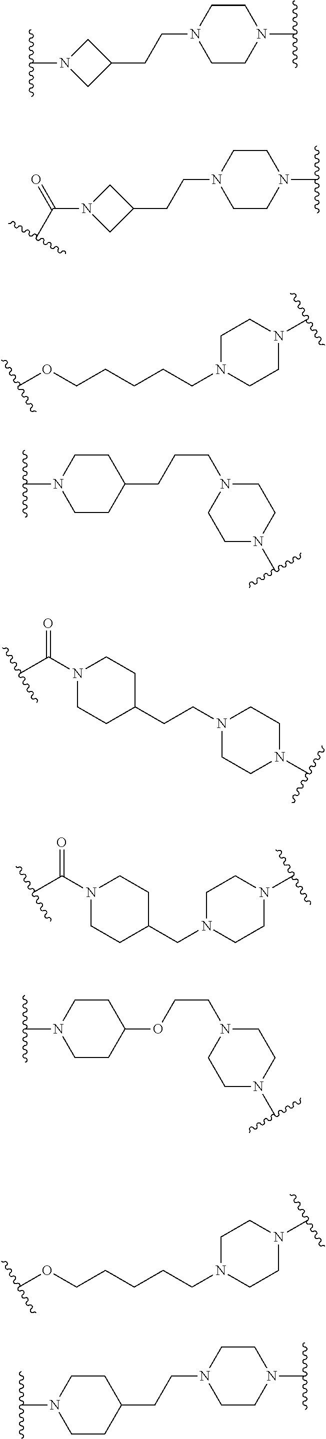

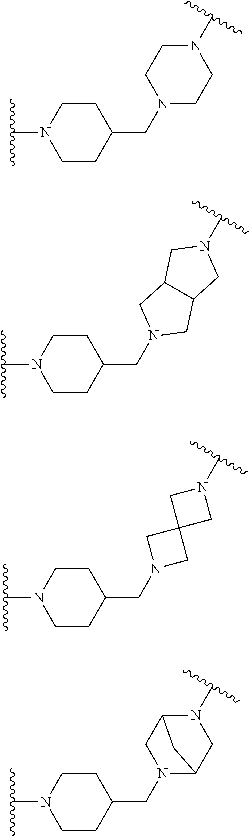 [SMILES: *N1CCC(CN2CCN(*)CC2)CC1 ; *N1CCC(CN2CC3CN(*)CC3C2)CC1 ; *N1CCC(CN2CC3(CN(*)C3)C2)CC1 ; *N1CCC(CN2CC3CC2CN3*)CC1]

In one embodiment, Linker, or the moiety —X-Het¹-, is selected from the group consisting of:

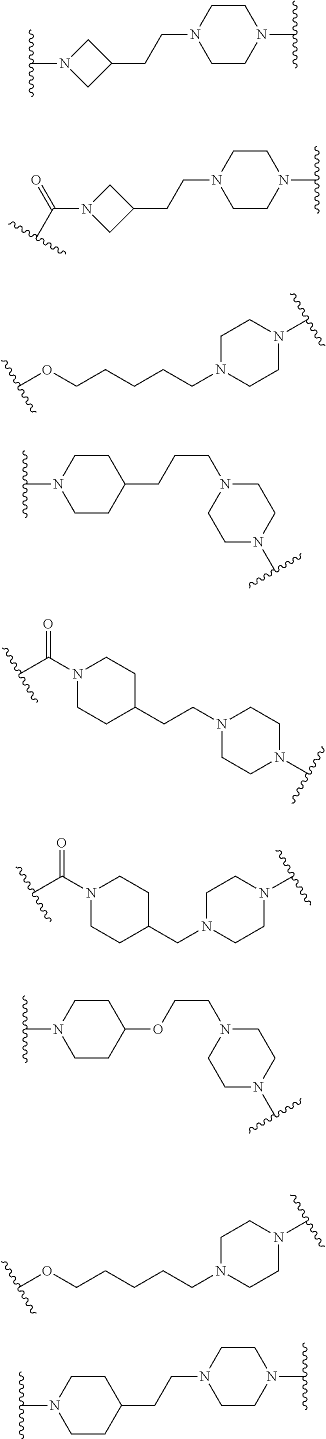

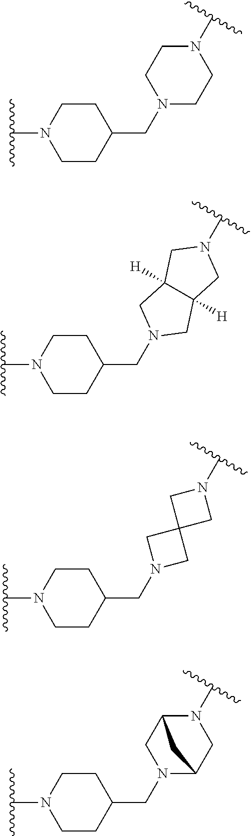 [SMILES: *N1CCC(CN2CCN(*)CC2)CC1 ; *N1CCC(CN2C[C@H]3CN(*)C[C@H]3C2)CC1 ; *N1CCC(CN2CC3(CN(*)C3)C2)CC1 ; *N1CCC(CN2C[C@H]3C[C@@H]2CN3*)CC1]

In one embodiment, Linker, or the moiety —X-Het¹-, is selected from the group consisting of:

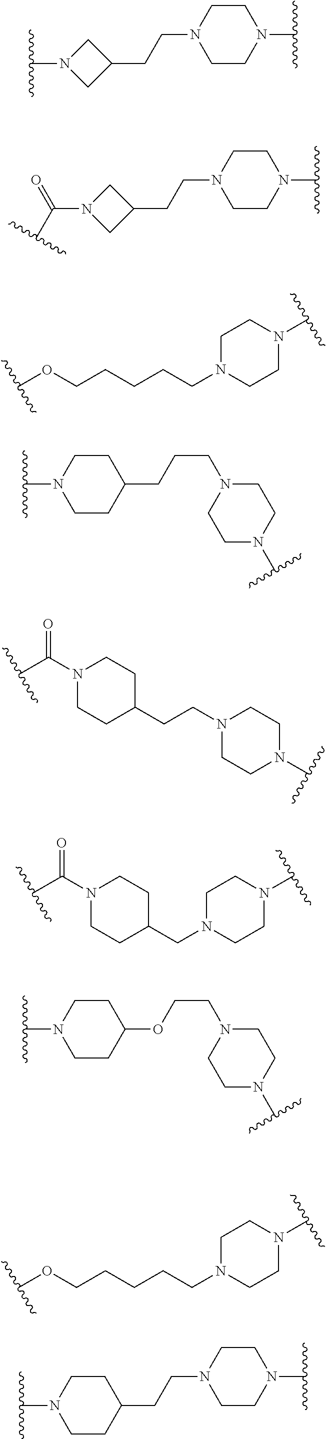

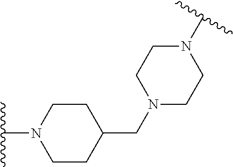 [SMILES: *N1CCC(CN2CCN(*)CC2)CC1]

In one embodiment, Linker, or the moiety —X-Het¹, is selected from the group consisting of:

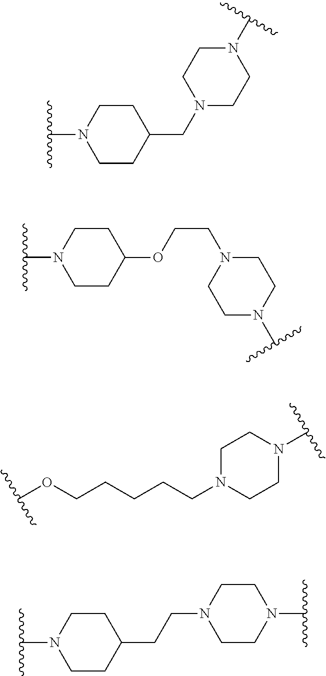 [SMILES: *N1CCC(CN2CCN(*)CC2)CC1 ; *N1CCC(OCCN2CCN(*)CC2)CC1 ; *OCCCCCN1CCN(*)CC1 ; *N1CCC(CCN2CCN(*)CC2)CC1]

In one embodiment, there is provided a compound of Formula (I), or a pharmaceutically acceptable salt thereof, wherein:

A and G are both CF or are both CH or A is CH and G is N;

D and E are both CH or are both N;

R¹ is H;

R² is H;

or R¹ and R² together with the carbon to which they are attached form carbonyl;

R³ is H;

R⁴ is H or OMe;

R⁶ is H, Me or F;

R⁷ is H, Me or F;

or R⁶ and R⁷ taken together with the carbon atom to which they are attached form a cyclopropyl ring or an oxetanyl ring;

R⁸ is H, Me, F, CH₂F, CHF₂, CF₃, CN, CH₂CN, CH₂OMe, CH₂OH, C(O)OH, C(O)OMe or SO₂Me;

Linker is represented by the moiety —X—[Y]ₙ-Het¹-, wherein:

X is selected from the group consisting of -Het²-C₁₋₆alkylene, —C₁₋₆alkenylene, —O-Het²-C₁₋₆alkylene, —C₁₋₆ alkylene and —O-Cyc-C₁₋₆alkylene, wherein one or two —CH₂— units in the alkylene chain is independently replaced with —O—, —NH— or —NMe-;

W is selected from -Het³-C₁₋₆ alkylene;

Het¹ is a nitrogen containing monocyclic or bicyclic heterocycloalkyl group;

Het² is a nitrogen containing monocyclic or bicyclic heterocycloalkyl group;

Het³ is a nitrogen containing monocyclic or bicyclic heterocycloalkyl group;

Cyc is C₃₋₆cycloalkyl;

p is 0 or 1;

wherein heterocycloalkyl is optionally substituted with 1 or 2 oxo substituents.

In one embodiment, there is provided a compound of Formula (I), or a pharmaceutically acceptable salt thereof, wherein:

A and G are both CF or are both CH or A is CH and G is N;

D and E are both CH or are both N;

R¹ is H;

R² is H;

or R¹ and R² together with the carbon to which they are attached form carbonyl;

R³ is H;

R⁴ is H or OMe;

R⁶ is Me;

R⁷ is Me;

R⁸ is F;

Linker is represented by the moiety —X—[W]ₚ-Het¹-, wherein:

X is selected from the group consisting of -Het²-C₁₋₆ alkylene-, —C₁₋₆ alkylene-, —O-Het²-C₁₋₃alkylene and —O-Cyc-C₁₋₃alkylene, wherein one or two —CH₂— units in the alkylene chain is independently replaced with —O— or —NMe-;

W is selected from -Het³-C₁₋₃alkylene;

Het¹ is a nitrogen containing monocyclic or bicyclic heterocycloalkyl group;

Het² is a nitrogen containing monocyclic or bicyclic heterocycloalkyl group;

Het³ is a nitrogen containing monocyclic or bicyclic heterocycloalkyl group;

Cyc is C₃₋₆cycloalkyl;

p is 0 or 1;

wherein heterocycloalkyl is optionally substituted with 1 or 2 oxo substituents.

In one embodiment, Het¹ is selected from the group consisting of piperidin-1-yl, piperazin-1-yl, 3,9-diazaspiro[5.5]undecan-3-yl, 7-oxa-3,10-diazaspiro[5.6]dodecan-3-yl, 3-oxopiperazin-1-yl, 2,7-diazaspiro[3.5]nonan-7-yl, 2,6-diazaspiro[3.3]heptan-2-yl, azetidin-1-yl and 2,5-diazabicyclo[2.2.1]heptan-2-yl.

In one embodiment, Het² is selected from the group consisting of piperidin-4-yl, 3,9-diazaspiro[5.5]undecan-3-yl, 7-oxa-3,10-diazaspiro[5.6]dodecan-10-yl, 7-azaspiro[3.5]nonan-2-yl, 2-oxo-3,9-diazaspiro[5.5]undecan-3-yl, 2,7-diazaspiro[3.5]nonan-2-yl, 6-azaspiro[2.5]octan-1-yl, azetidin-3-yl and 3-azaspiro[5.5]undecan-3-yl.

In one embodiment, Het³ is selected from the group consisting of piperidin-4-yl, piperazin-1-yl and azetidin-1yl.

In one embodiment, Cyc is cyclobutyl.

In one embodiment, there is provided a compound of Formula (I), or a pharmaceutically acceptable salt thereof, wherein:

A and G are both CF or are both CH;

D and E are both CH or are both N;

R¹ is H;

R² is H;

or R¹ and R² together with the carbon to which they are attached form carbonyl;

R³ is H or OMe;

R⁴ is H or OMe;

R⁶ is H, Me or F;

R⁷ is H, Me or F;

or R⁶ and R⁷ taken together with the carbon atom to which they are attached form a cyclopropyl ring or an oxetanyl ring;

R⁸ is H, Me, F, CH₂F, CHF₂, CF₃, CN, CH₂CN, CH₂OMe, CH₂OH, C(O)OH, C(O)OMe or SO₂Me;

Linker is represented by —X-Het¹-, wherein X is selected from -Het²-C₁₋₆ alkylene or —C₁₋₆ alkylene wherein one or two —CH₂— units in the alkylene chain is replaced with —O—; Het¹ is a nitrogen containing monocyclic heterocycloalkyl group; and Het² is a nitrogen containing monocyclic heterocycloalkyl group.

In one embodiment, -Het¹- is piperazinyl.

In one embodiment, -Het²- is piperidinyl.

In one embodiment, Linker, or the moiety —X-Het¹-, is selected from the group consisting of:

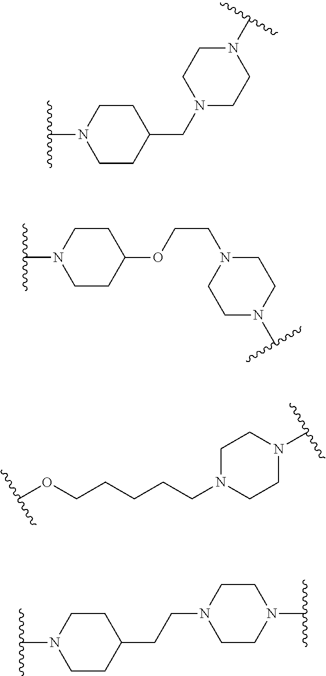 [SMILES: *N1CCC(CN2CCN(*)CC2)CC1 ; *N1CCC(OCCN2CCN(*)CC2)CC1 ; *OCCCCCN1CCN(*)CC1 ; *N1CCC(CCN2CCN(*)CC2)CC1]

In one embodiment, there is provided a compound of Formula (I), or a pharmaceutically acceptable salt thereof, wherein:

A and G are both CF or are both CH;

D and E are both CH or are both N;

R¹ is H;

R² is H;

or R¹ and R² together with the carbon to which they are attached form carbonyl;

R³ is H;

R⁴ is H;

R⁶ is Me;

R⁷ is Me;

R⁸ is F;

Linker is represented by —X-Het¹-, wherein X is selected from -Het²-C₁₋₆ alkylene- or —C₁₋₆ alkylene- wherein one or two —CH₂— units in the alkylene chain is replaced with —O—; Het¹ is a nitrogen containing monocyclic heterocycloalkyl group; and Het² is a nitrogen containing monocyclic heterocycloalkyl group.

In one embodiment, -Het¹- is piperazinyl.

In one embodiment, -Het²- is piperidinyl.

In one embodiment, Linker, or the moiety —X-Het¹-, is selected from the group consisting of:

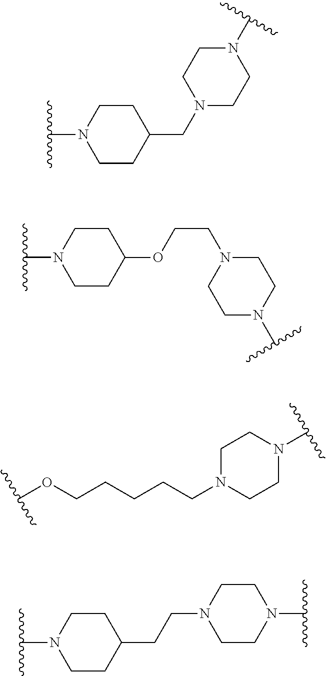 [SMILES: *N1CCC(CN2CCN(*)CC2)CC1 ; *N1CCC(OCCN2CCN(*)CC2)CC1 ; *OCCCCCN1CCN(*)CC1 ; *N1CCC(CCN2CCN(*)CC2)CC1]

In one embodiment there is provided a compound of Formula (I), or a pharmaceutically acceptable salt thereof, wherein the compound is selected from the group consisting of:
3-[5-[4-[[1-[5-[(1R,3R)-2-(2-Fluoro-2-methyl-propyl)-3-methyl-1,3,4,9-tetrahydropyrido[3,4-b]indol-1-yl]pyrimidin-2-yl]-4-piperidyl]methyl]piperazin-1-yl]-1-oxo-isoindolin-2-yl]piperidine-2,6-dione;
3-[5-[4-[2-[1-[5-[(1R,3R)-2-(2-Fluoro-2-methyl-propyl)-3-methyl-1,3,4,9-tetrahydropyrido[3,4-b]indol-1-yl]pyrimidin-2-yl]-4-piperidyl]ethyl]piperazin-1-yl]-1-oxo-isoindolin-2-yl]piperidine-2,6-dione;
2-[2,6-Dioxo3-piperidyl]-5-[4-[[1-[5-[(1R,3R)-2-(2-fluoro-2-methyl-propyl)-3-methyl-1,3,4,9-tetrahydropyrido[3,4-b]indol-1-yl]pyrimidin-2-yl]-4-piperidyl]methyl]piperazin-1-yl]isoindoline-1,3-dione formate;
3-[5-[4-[2-[[1-[5-[(1R,3R)-2-(2-Fluoro-2-methyl-propyl)-3-methyl-1,3,4,9-tetrahydropyrido[3,4-b]indol-1-yl]pyrimidin-2-yl]-4-piperidyl]oxy]ethyl]piperazin-1-yl]-1-oxo-isoindolin-2-yl]piperidine-2,6-dione;
3-[5-[4-[5-[3,5-Difluoro-4-[(1R,3R)-2-(2-fluoro-2-methyl-propyl)-3-methyl-1,3,4,9-tetrahydropyrido[3,4-b]indol-1-yl]phenoxy]pentyl]piperazin-1-yl]-1-oxo-isoindolin-2-yl]piperidine-2,6-dione;
3-{5-[4-({4-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]piperazin-1-yl}methyl)piperidin-1-yl]-1-oxo-1,3-dihydro-2H-isoindol-2-yl}piperidine-2,6-dione;
3-(5-{9-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]-3,9-diazaspiro[5.5]undecan-3-yl}-1-oxo-1,3-dihydro-2H-isoindol-2-yl)piperidine-2,6-dione;
3-(5-{4-[3-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)propyl]piperazin-1-yl}-1-oxo-1,3-dihydro-2H-isoindol-2-yl)piperidine-2,6-dione;
3-(5-{4-[(9-{5-[(1R,3R)-2-(2-fluoro-2-methylpropyl)-3-methyl-2,3,4,9-tetrahydro-1H-beta-carbolin-1-yl]pyrimidin-2-yl}-3,9-diazaspiro[5.5]undecan-3-yl)methyl]piperidin-1-yl}-1-oxo-1,3-dihydro-2H-isoindol-2-yl)piperidine-2,6-dione;
3-(5-{4-[2-(9-{5-[(1R,3R)-2-(2-fluoro-2-methylpropyl)-3-methyl-2,3,4,9-tetrahydro-1H-beta-carbolin-1-yl]pyrimidin-2-yl}-3,9-diazaspiro[5.5]undecan-3-yl)ethyl]piperidin-1-yl}-1-oxo-1,3-dihydro-2H-isoindol-2-yl)piperidine-2,6-dione;
3-(5-{9-[2-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl]-3,9-diazaspiro[5.5]undecan-3-yl}-1-oxo-1,3-dihydro-2H-isoindol-2-yl)piperidine-2,6-dione;
3-(5-{4-[2-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl]piperazin-1-yl}-7-methoxy-1-oxo-1,3-dihydro-2H-isoindol-2-yl)piperidine-2,6-dione;
3-(5-{4-[3-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)propyl]piperazin-1-yl}-7-methoxy-1-oxo-1,3-dihydro-2H-isoindol-2-yl)piperidine-2,6-dione;
3-{5-[4-({1-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]piperidin-4-yl}methyl)piperazin-1-yl]-1-oxo-1,3-dihydro-2H-isoindol-2-yl}piperidine-2,6-dione;
3-(5-{4-[2-(1-{5-[(1R,3R)-3-methyl-2-(2,2,2-trifluoroethyl)-2,3,4,9-tetrahydro-1H-beta-carbolin-1-yl]pyrimidin-2-yl}piperidin-4-yl)ethyl]piperazin-1-yl}-1-oxo-1,3-dihydro-2H-isoindol-2-yl)piperidine-2,6-dione;
3-(5-{4-[(3-{[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl](methyl)amino}azetidin-1-yl)methyl]piperidin-1-yl}-1-oxo-1,3-dihydro-2H-isoindol-2-yl)piperidine-2,6-dione;
3-(5-{4-[2-(3-{5-[(1R,3R)-2-(2-fluoro-2-methylpropyl)-3-methyl-2,3,4,9-tetrahydro-1H-beta-carbolin-1-yl]pyrimidin-2-yl}-7-oxa-3,10-diazaspiro[5.6]dodecan-10-yl)ethyl]piperidin-1-yl}-1-oxo-1,3-dihydro-2H-isoindol-2-yl)piperidine-2,6-dione;
3-(5-{4-[(3-{5-[(1R,3R)-2-(2-fluoro-2-methylpropyl)-3-methyl-2,3,4,9-tetrahydro-1H-beta-carbolin-1-yl]pyrimidin-2-yl}-7-oxa-3,10-diazaspiro[5.6]dodecan-10-yl)methyl]piperidin-1-yl}-1-oxo-1,3-dihydro-2H-isoindol-2-yl)piperidine-2,6-dione;
3-(5-{10-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]-7-oxa-3,10-diazaspiro[5.6]dodecan-3-yl}-1-oxo-1,3-dihydro-2H-isoindol-2-yl)piperidine-2,6-dione;
3-(5-{10-[2-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl]-7-oxa-3,10-diazaspiro[5.6]dodecan-3-yl}-1-oxo-1,3-dihydro-2H-isoindol-2-yl)piperidine-2,6-dione;
3-(5-{9-[(1-{6-[(1S,3R)-2-(2-fluoro-2-methylpropyl)-3-methyl-2,3,4,9-tetrahydro-1H-beta-carbolin-1-yl]pyridin-3-yl}piperidin-4-yl)methyl]-3,9-diazaspiro[5.5]undecan-3-yl}-1-oxo-1,3-dihydro-2H-isoindol-2-yl)piperidine-2,6-dione;
3-(5-{9-[(7-{5-[(1R,3R)-2-(2-fluoro-2-methylpropyl)-3-methyl-2,3,4,9-tetrahydro-1H-beta-carbolin-1-yl]pyrimidin-2-yl}-7-azaspiro[3.5]nonan-2-yl)methyl]-3,9-diazaspiro[5.5]undecan-3-yl}-1-oxo-1,3-dihydro-2H-isoindol-2-yl)piperidine-2,6-dione;
3-[5-(9-{2-[(1S,3r)-3-({5-[(1R,3R)-2-(2-fluoro-2-methylpropyl)-3-methyl-2,3,4,9-tetrahydro-1H-beta-carbolin-1-yl]pyrimidin-2-yl}oxy)cyclobutyl]ethyl}-3,9-diazaspiro[5.5]undecan-3-yl)-1-oxo-1,3-dihydro-2H-isoindol-2-yl]piperidine-2,6-dione;
3-(5-{9-[5-({5-[(1R,3R)-2-(2-fluoro-2-methylpropyl)-3-methyl-2,3,4,9-tetrahydro-1H-beta-carbolin-1-yl]pyrimidin-2-yl}oxy)pentyl]-3,9-diazaspiro[5.5]undecan-3-yl}-1-oxo-1,3-dihydro-2H-isoindol-2-yl)piperidine-2,6-dione;
3-(5-{4-[2-(9-{5-[(1R,3R)-2-(2-fluoro-2-methylpropyl)-3-methyl-2,3,4,9-tetrahydro-1H-beta-carbolin-1-yl]pyrimidin-2-yl}-2-oxo-3,9-diazaspiro[5.5]undecan-3-yl)ethyl]piperazin-1-yl}-1-oxo-1,3-dihydro-2H-isoindol-2-yl)piperidine-2,6-dione;
3-(5-{4-[2-(9-{5-[(1R,3R)-2-(2-fluoro-2-methylpropyl)-3-methyl-2,3,4,9-tetrahydro-1H-beta-carbolin-1-yl]pyrimidin-2-yl}-3,9-diazaspiro[5.5]undecan-3-yl)ethyl]-3-oxopiperazin-1-yl}-1-oxo-1,3-dihydro-2H-isoindol-2-yl)piperidine-2,6-dione;
3-(5-{4-[(7-{5-[(1R,3R)-2-(2-fluoro-2-methylpropyl)-3-methyl-2,3,4,9-tetrahydro-1H-beta-carbolin-1-yl]pyrimidin-2-yl}-7-azaspiro[3.5]nonan-2-yl)methyl]piperazin-1-yl}-1-oxo-1,3-dihydro-2H-isoindol-2-yl)piperidine-2,6-dione;
3-(5-{2-[(7-{5-[(1R,3R)-2-(2-fluoro-2-methylpropyl)-3-methyl-2,3,4,9-tetrahydro-1H-beta-carbolin-1-yl]pyrimidin-2-yl}-7-azaspiro[3.5]nonan-2-yl)methyl]-2,7-diazaspiro[3.5]nonan-7-yl}-1-oxo-1,3-dihydro-2H-isoindol-2-yl)piperidine-2,6-dione;
3-(5-{4-[(7-{5-[(1R,3R)-2-(2-fluoro-2-methylpropyl)-3-methyl-2,3,4,9-tetrahydro-1H-beta-carbolin-1-yl]pyrimidin-2-yl}-2,7-diazaspiro[3.5]nonan-2-yl)methyl]piperidin-1-yl}-1-oxo-1,3-dihydro-2H-isoindol-2-yl)piperidine-2,6-dione;
3-(5-{6-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]-2,6-diazaspiro[3.3]heptan-2-yl}-1-oxo-1,3-dihydro-2H-isoindol-2-yl)piperidine-2,6-dione;
3-(5-{4-[(6-{5-[(1R,3R)-2-(2-fluoro-2-methylpropyl)-3-methyl-2,3,4,9-tetrahydro-1H-beta-carbolin-1-yl]pyrimidin-2-yl}-6-azaspiro[2.5]octan-1-yl)methyl]piperazin-1-yl}-1-oxo-1,3-dihydro-2H-isoindol-2-yl)piperidine-2,6-dione;
3-[5-(3-{[2-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl](methyl)amino}azetidin-1-yl)-1-oxo-1,3-dihydro-2H-isoindol-2-yl]piperidine-2,6-dione;
3-(5-{(1R,4R)-5-[3-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)propyl]-2,5-diazabicyclo[2.2.1]heptan-2-yl}-1-oxo-1,3-dihydro-2H-isoindol-2-yl)piperidine-2,6-dione;
3-(5-{4-[3-(1-{5-[(1R,3R)-3-methyl-2-(2,2,2-trifluoroethyl)-2,3,4,9-tetrahydro-1H-beta-carbolin-1-yl]pyrimidin-2-yl}piperidin-4-yl)propyl]piperazin-1-yl}-1-oxo-1,3-dihydro-2H-isoindol-2-yl)piperidine-2,6-dione;
3-(5-{4-[(1-{5-[(1R,3R)-3-methyl-2-(2,2,2-trifluoroethyl)-2,3,4,9-tetrahydro-1H-beta-carbolin-1-yl]pyrimidin-2-yl}piperidin-4-yl)methyl]piperazin-1-yl}-1-oxo-1,3-dihydro-2H-isoindol-2-yl)piperidine-2,6-dione;
3-(5-{4-[1-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl]piperazin-1-yl}-1-oxo-1,3-dihydro-2H-isoindol-2-yl)piperidine-2,6-dione;
3-(5-{4-[2-(1-{5-[(1R,3R)-2-(2-fluoro-2-methylpropyl)-3-methyl-2,3,4,9-tetrahydro-1H-beta-carbolin-1-yl]pyrimidin-2-yl}azetidin-3-yl)ethyl]piperazin-1-yl}-1-oxo-1,3-dihydro-2H-isoindol-2-yl)piperidine-2,6-dione;
3-[5-(4-{3-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)oxy]propyl}piperazin-1-yl)-1-oxo-1,3-dihydro-2H-isoindol-2-yl]piperidine-2,6-dione;
3-(5-{4-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]piperazin-1-yl}-7-methoxy-1-oxo-1,3-dihydro-2H-isoindol-2-yl)piperidine-2,6-dione;
3-(5-{4-[5-({5-[(1R,3R)-2-(2-fluoro-2-methylpropyl)-3-methyl-2,3,4,9-tetrahydro-1H-beta-carbolin-1-yl]pyrimidin-2-yl}oxy)pentyl]piperazin-1-yl}-1-oxo-1,3-dihydro-2H-isoindol-2-yl)piperidine-2,6-dione; and
3-[5-(4-{[9-({5-[(1R,3R)-2-(2-fluoro-2-methylpropyl)-3-methyl-2,3,4,9-tetrahydro-1H-beta-carbolin-1-yl]pyrimidin-2-yl}oxy)-3-azaspiro[5.5]undecan-3-yl]methyl}piperidin-1-yl)-1-oxo-1,3-dihydro-2H-isoindol-2-yl]piperidine-2,6-dione.

The compounds of Formula (I) have two or more chiral centres and it will be recognised that the compounds of Formula (I) may be prepared, isolated and/or supplied with or without the presence, in addition, of one or more of the other possible enantiomeric and/or diastereomeric isomers of the compounds of Formula (I) in any relative proportions. The preparation of enantioenriched/enantiopure and/or diastereoenriched/diastereopure compounds may be carried out by standard techniques of organic chemistry that are well known in the art, for example by synthesis from enantioenriched or enantiopure starting materials, use of an appropriate enantioenriched or enantiopure catalyst during synthesis, and/or by resolution of a racemic or partially enriched mixture of stereoisomers, for example via chiral chromatography.

For use in a pharmaceutical context it may be preferable to provide a compound of Formula (I) or a pharmaceutically acceptable salt thereof without large amounts of the other stereoisomeric forms being present.

Accordingly, in one embodiment there is provided a composition comprising a compound of Formula (I) or a pharmaceutically acceptable salt thereof, optionally together with one or more of the other stereoisomeric forms of the compound of Formula (I) or pharmaceutically acceptable salt thereof, wherein the compound of Formula (I) or pharmaceutically acceptable salt thereof is present within the composition with a diastereomeric excess (% de) of ≥90%.

In a further embodiment the % de in the above-mentioned composition is 95%.

In a further embodiment the % de in the above-mentioned composition is 98%.

In a further embodiment the % de in the above-mentioned composition is 99%.

In a further embodiment there is provided a composition comprising a compound of Formula (I) or a pharmaceutically acceptable salt thereof, optionally together with one or more of the other stereoisomeric forms of the compound of Formula (I) or pharmaceutically acceptable salt thereof, wherein the compound of Formula (I) or pharmaceutically acceptable salt thereof is present within the composition with an enantiomeric excess (% ee) of ≥90%.

In a further embodiment the % ee in the above-mentioned composition is 95%.

In a further embodiment the % ee in the above-mentioned composition is 98%.

In a further embodiment the % ee in the above-mentioned composition is 99%.

In a further embodiment there is provided a composition comprising a compound of Formula (I) or a pharmaceutically acceptable salt thereof, optionally together with one or more of the other stereoisomeric forms of the compound of Formula (I), or pharmaceutically acceptable salt thereof, wherein the compound of Formula (I), or pharmaceutically acceptable salt thereof is present within the composition with an enantiomeric excess (% ee) of ≥90% and a diastereomeric excess (% de) of ≥90%.

In further embodiments of the above-mentioned composition the % ee and % de may take any combination of values as listed below:
The % ee is ≤5% and the % de is ≥80%.
The % ee is ≤5% and the % de is ≥90%.
The % ee is ≤5% and the % de is ≥95%.
The % ee is ≤5% and the % de is ≥98%.
The % ee is ≥95% and the % de is ≥95%.
The % ee is ≥98% and the % de is ≥98%.
The % ee is ≥99% and the % de is ≥99%.

In a further embodiment there is provided a pharmaceutical composition which comprises a compound of the Formula (I), or a pharmaceutically acceptable salt thereof, in association with a pharmaceutically acceptable excipient.

In one embodiment there is provided a pharmaceutical composition which comprises a compound of the Formula (I), or a pharmaceutically acceptable salt thereof, in association with a pharmaceutically acceptable excipient, optionally further comprising one or more of the other stereoisomeric forms of the compound of Formula (I), or pharmaceutically acceptable salt thereof, wherein the compound of Formula (I), or pharmaceutically acceptable salt thereof is present within the composition with an enantiomeric excess (% ee) of 90%.

In a further embodiment the % ee in the above-mentioned composition is ≥95%.

In a further embodiment the % ee in the above-mentioned composition is ≥98%.

In a further embodiment the % ee in the above-mentioned composition is ≥99%.

In one embodiment there is provided a pharmaceutical composition which comprises a compound of the Formula (I), or a pharmaceutically acceptable salt thereof, in association with a pharmaceutically acceptable excipient, optionally further comprising one or more of the other stereoisomeric forms of the compound of Formula (I), or pharmaceutically acceptable salt thereof, wherein the compound of Formula (I), or pharmaceutically acceptable salt thereof is present within the composition with a diastereomeric excess (% de) of 90%.

In a further embodiment the % de in the above-mentioned composition is ≥95%.

In a further embodiment the % de in the above-mentioned composition is ≥98%.

In a further embodiment the % de in the above-mentioned composition is ≥99%.

In one embodiment there is provided a pharmaceutical composition which comprises a compound of the Formula (I), or a pharmaceutically acceptable salt thereof, in association with a pharmaceutically acceptable excipient, optionally further comprising one or more of the other stereoisomeric forms of the compound of Formula (I), or pharmaceutically acceptable salt thereof, wherein the compound of Formula (I), or pharmaceutically acceptable salt thereof is present within the composition with an enantiomeric excess (% ee) of 90% and a diastereomeric excess (% de) of ≥90%.

In further embodiments of the above-mentioned pharmaceutical composition the % ee and % de may take any combination of values as listed below:
The % ee is ≥95% and the % de is ≥95%.
The % ee is ≥98% and the % de is ≥98%.
The % ee is ≥99% and the % de is ≥99%.

The compounds of Formula (I), and pharmaceutically acceptable salts thereof may be prepared, used or supplied in amorphous form, crystalline form, or semicrystalline form and any given compound of Formula (I), or pharmaceutically acceptable salt thereof may be capable of being formed into more than one crystalline/polymorphic form, including hydrated (e.g. hemi-hydrate, a mono-hydrate, a di-hydrate, a tri-hydrate or other stoichiometry of hydrate) and/or solvated forms. It is to be understood that the present specification encompasses any and all such solid forms of the compound of Formula (I), and pharmaceutically acceptable salts thereof.

In further embodiments there is provided a compound of Formula (I) which is obtainable by the methods described in the ‘Examples’ section hereinafter.

The present specification is intended to include all isotopes of atoms occurring in the present compounds. Isotopes will be understood to include those atoms having the same atomic number but different mass numbers.

For the avoidance of doubt it is to be understood that where in this specification a group is qualified by ‘hereinbefore defined’ or ‘defined herein’ the said group encompasses the first occurring and broadest definition as well as each and all of the alternative definitions for that group.

Another aspect of the present specification provides a process for preparing a compound of the Formula (I), or a pharmaceutically acceptable salt thereof. A suitable process is illustrated by the following representative process variants in which, unless otherwise stated, A, D, E, G, Linker and R¹ to R⁸ have any of the meanings defined hereinbefore. Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

General Scheme

Compounds of Formula (I) may be made by, for example:

a) Reductive aminatin reaction of an aldehyde compound of Formula (II) with an amine compound of Formula (III) under conditions known in the art as suitable reductive amination (such as in the presence of a suitable amine reduction reagent (such as sodium triacetoxyborohydride) and in a suitable solvent (for example DCM) and a suitable temperature (such as room temperature). In a certain aspect, where there is a nitrogen in the linker group ( [symbol] ), the nitrogen is protected with a protecting group (such as Boc or Cbz) that may be removed under conditions known in the art.

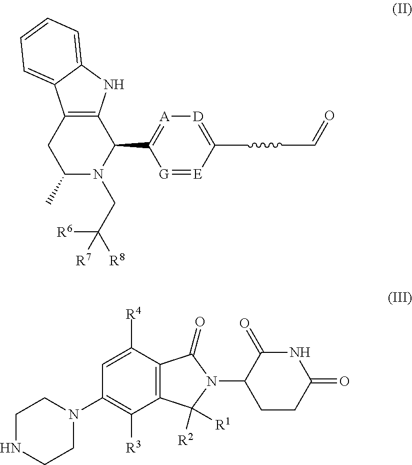

A, D, G, E, R¹, R², R³, R⁴, R⁶, R⁷, R⁸ are as defined herein and “ [symbol] ” in Formula (II) represents the part of the Linker which is not present in Formula (III) and is as defined herein.

b) Amine alkylation reaction of a compound of Formula (IV) where LG is a leaving group (e.g. a tosyl group or a halide such as bromide) with an amine compound of Formula (III) under conditions known in the art as suitable amine alkylation reactions (such as in the presence of a suitable base (for example potassium carbonate) and in a suitable solvent (for example DMF) and a suitable temperature (such as 50° C.)). In a certain aspect, where there is a nitrogen in the linker group ( [symbol] ), the nitrogen is protected with a protecting group (such as Boc or Cbz) that may be removed under conditions known in the art.

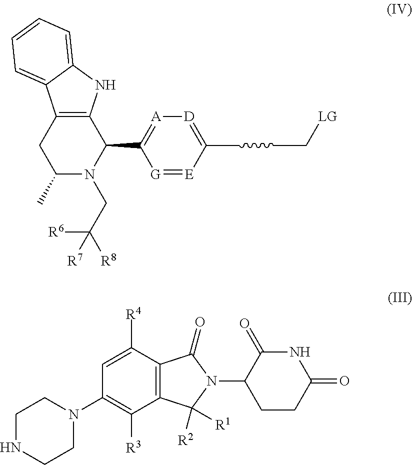

A, D, G, E, R¹, R², R³, R⁴, R⁶, R⁷, R⁸ are as defined herein and “ [symbol] ” in Formula (IV) represents the part of the Linker which is not present in Formula (III) and is as defined herein.

c) Buchwald coupling reaction of a compound of Formula (V) where Y is a halide (such as bromide) with an amine compound of Formula (VI) under conditions known in the art as suitable Buchwald coupling reactions, such as in the presence of a suitable palladium catalyst (such as Pd(OAc)₂), a suitable ligand (such as BINAP), a suitable base (such as sodium carbonate), and in a suitable solvent (for example toluene) and a suitable temperature (such as 100° C.). In certain aspects, where there is a nitrogen in the linker group ( [symbol] ), the nitrogen is protected with a protecting group (such as Boc or Cbz) that may be removed under conditions known in the art.

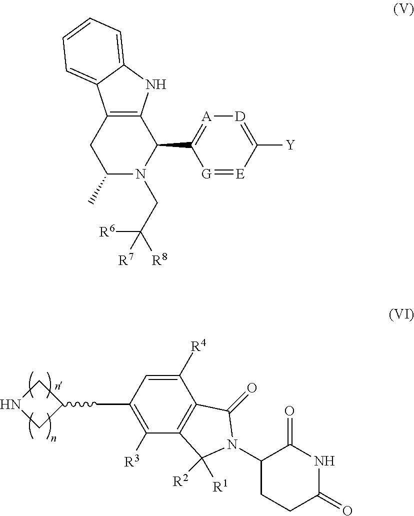

A, D, G, E, R¹, R², R³, R⁴, R⁶, R⁷, R⁸ are as defined herein, n is 1 or 2 and n′ is 1 or 2, and “ [symbol] ” in Formula (VI) represents the part of the Linker which is not present in Formula (V) and is as defined herein.

d) Alkylation of a suitable amine of Formula (VII) with a compound of Formula (VIII) where LG is a leaving group known in the art, for example halides (such as bromide), in a suitable solvent (for example acetonitrile) in the presence of a suitable base (for example potassium carbonate) and at a suitable temperature (such as 80-90° C.).

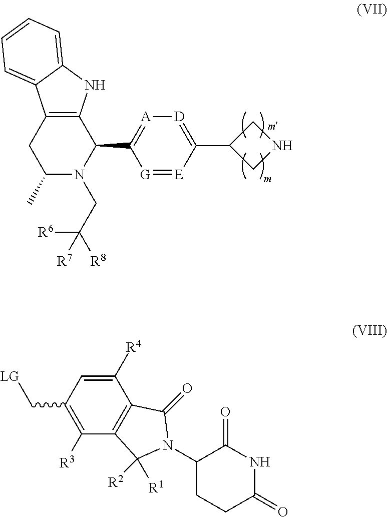

A, D, G, E, R¹, R², R³, R⁴, R⁶, R⁷, R⁸ are as defined herein, m is 1 or 2 and m′ is 1 or 2, and “ [symbol] ” in Formula (VII) represents the part of the Linker which is not present in Formula (VIII), and vice versa, and is as defined herein.

e) Amination of aryl halide compounds of Formula (IX) wherein Z is chloride, bromide, or iodide, with amine compounds of Formula (VII) under suitable Buckwald reaction conditions using palladium catalyst (such as Pd(OAc)₂ or Pd-PEPPSI-IHept^{Cl}), a suitable ligand (such as BINAP), a suitable base (such as sodium carbonate or cesium carbonate), in a suitable solvent (such as tolune or 1,4-dioxane). Another suitable reaction is nucleophilic aromatic substitution reaction of compounds of Formula (VII) with compounds of Formula (IX), wherein Z is fluoride, chloride, or bromide, using suitable base (such as DIPEA) in a suitable solvent (such as NMP) and heating to a suitable temperature (such as 140° C.).

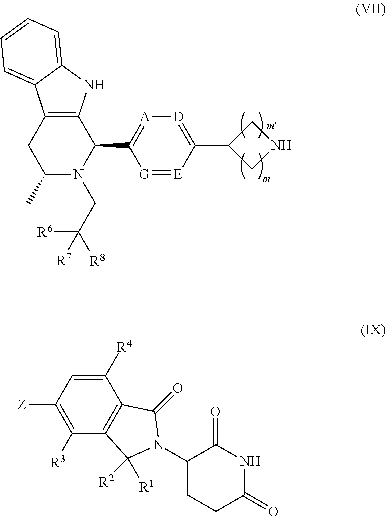

A, D, G, E, R¹, R², R³, R⁴, R⁶, R⁷, R⁸ are as defined herein and m is 1 or 2 and m′ is 1 or 2, and “ [symbol] ” in Formula (VII) represents the part of the Linker which is not present in Formula (IX) and is as defined herein.

[symbol]

f) Double deprotection of tert-butyl carbamate compounds of Formula (X) and acetal compounds of Formula (XI) in formic acid at a suitable temperature (such as 40° C.), followed by evaporation to dryness and dissolution in a suitable solvent (such as DCM) and addition of a suitable reducing agent (such as sodium triacetoxyborohydride).

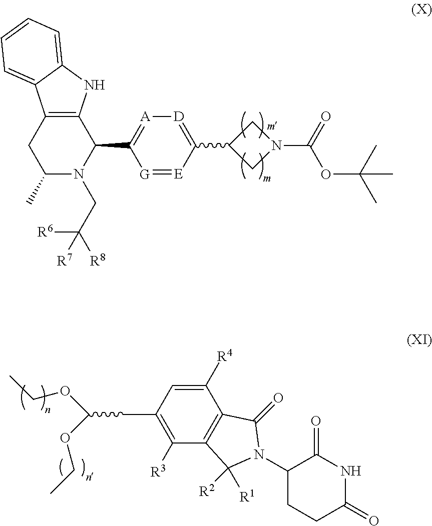

A, D, G, E, R¹, R², R³, R⁴, R⁶, R⁷, R⁸ are as defined herein and m is 1 or 2 and m′ is 1 or 2 and n is 0 or 1 or 2 or 3 and n′ is 0 or 1 or 2 or 3, and “ [symbol] ” in Formula (X) represents the part of the Linker which is not present in Formula (XI) and “ [symbol] ” in Formula (XI) represents the part of the Linker which is not present in Formula (X) and is as defined herein.

g) Double deprotection of tert-butyl carbamate compounds of Formula (XII) and acetal compounds of Formula (XIII) in formic acid at a suitable temperature (such as 40° C.), followed by evaporation to dryness and dissolution in a suitable solvent (such as DCM) and addition of a suitable reducing agent (such as sodium triacetoxyborohydride).

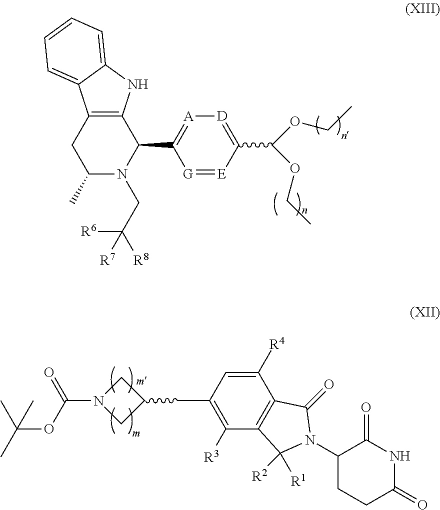

A, D, G, E, R¹, R², R³, R⁴, R⁶, R⁷, R⁸ are as defined herein and m is 1 or 2 and m′ is 1 or 2 and n is 0 or 1 or 2 or 3 and n′ is 0 or 1 or 2 or 3, and “ [symbol] ” in Formula (X) represents the part of the Linker which is not present in Formula (XI) and “ [symbol] ” in Formula (XI) represents the part of the Linker which is not present in Formula (X) and is as defined herein.

Compounds of Formula (II), Formula (IV), Formula (V), Formula (VII), Formula (X) and Formula (XIII) may be prepared in reference to the procedures described in WO2018019793, herein incorporated by reference, for those having ordinary skill in the art.

Compounds of Formula (III), Formula (VI), Formula (VIII), Formula (IX), Formula (XI) and Formula (XII) may be prepared in reference to the procedures described in WO2018071606, WO2018140809, WO2018102725, and US20180228907, herein incorporated by reference, for those having ordinary skill in the art.

It is to be understood that other permutations of the process steps in the process variants described above are also possible.

When a pharmaceutically acceptable salt of a compound of Formula (I) is required it may be obtained by, for example, reaction of said compound with a suitable acid or suitable base.

It will also be appreciated that, in some of the reactions mentioned hereinbefore, it may be necessary or desirable to protect any sensitive functionalities in the compounds. The instances where protection is necessary or desirable, and suitable methods for protection, are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy, it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an alkoxycarbonyl group such as a t-butoxycarbonyl group may be removed, for example, by treatment with a suitable acid as hydrochloric, sulphuric, formic, phosphoric or trifluoroacetic acid, and an arylmethoxycarbonyl group such as a benzyloxycarbonyl group may be removed, for example, by hydrogenation over a catalyst such as palladium-on-carbon, or by treatment with a Lewis acid, such as boron tris(trifluoroacetate). A suitable alternative protecting group for a primary amino group is, for example, a phthaloyl group, which may be removed by treatment with an alkylamine, for example dimethylaminopropylamine, or hydrazine.

The protecting groups may be removed at any convenient stage in the synthesis using conventional techniques well known in the chemical art.

Certain of the intermediates defined herein are novel and these are provided as further features of the specification.

### Biological Assays

The following assays were used to measure the effects of the compounds of the present specification.

### ERα Binding Assay

The ability of compounds to bind to isolated Estrogen Receptor Alpha Ligand binding domain (ER alpha-LBD (GST)) was assessed in competition assays using a LanthaScreen™ Time-Resolved Fluorescence Resonance Energy Transfer (TR-FRET) detection end-point. For the LanthaScreen TR-FRET endpoint, a suitable fluorophore (Fluormone ES2, ThermoFisher, Product code P2645) and recombinant human Estrogen Receptor alpha ligand binding domain, residues 307-554 (expressed and purified in-house) were used to measure compound binding. The assay principle is that ER alpha-LBD (GST) is added to a fluorescent ligand to form a receptor/fluorophore complex. A terbium-labelled anti-GST antibody (Product code PV3551) is used to indirectly label the receptor by binding to its GST tag, and competitive binding is detected by a test compound's ability to displace the fluorescent ligand, resulting in a loss of TR-FRET signal between the Th-anti-GST antibody and the tracer. The assay was performed as follows with all reagent additions carried out using the Beckman Coulter BioRAPTR FRD microfluidic workstation:
1. Acoustic dispense 120 nL of the test compound into a black low volume 384 well assay plates.
2. Prepare 1×ER alpha-LBD/Tb-anti-GST Ab in ES2 screening buffer and incubate for 15 minutes.
3. Dispense 6 μL of the 1×AR-LBD/Th-anti-GST Ab reagent into each well of the assay plate followed by 6 μL of Fluorophore reagent into each well of the assay plate
4. Cover the assay plate to protect the reagents from light and evaporation, and incubate at room temperature for 4 hours.
5. Excite at 337 nm and measure the fluorescent emission signal of each well at 490 nm and 520 nm using the BMG PheraSTAR.

Compounds were dosed directly from a compound source microplate containing serially diluted compound (4 wells containing 10 mM, 0.1 mM, 1 mM and 10 nM final compound respectively) to an assay microplate using the Labcyte Echo 550. The Echo 550 is a liquid handler that uses acoustic technology to perform direct microplate-to-microplate transfers of DMSO compound solutions and the system can be programmed to transfer multiple small nL volumes of compound from the different source plate wells to give the desired serial dilution of compound in the assay which is then back-filled to normalise the DMSO concentration across the dilution range.

In total 120 nL of compound plus DMSO were added to each well and compounds were tested in a 12-point concentration response format over a final compound concentration range of 10, 2.917, 1.042, 0.2083, 0.1, 0.0292, 0.0104, 0.002083, 0.001, 0.0002917, 0.0001042, and 0.00001 μM respectively. TR-FRET dose response data obtained with each compound was exported into a suitable software package (such as Origin or Genedata) to perform curve fitting analysis. Competitive ER alpha binding was expressed as an IC₅₀ value. This was determined by calculation of the concentration of compound that was required to give a 50% reduction in tracer compound binding to ER alpha-LBD.

### MCF-7 ER Degradation Assay

The ability of compounds to down-regulate Estrogen Receptor (ER) numbers was assessed in a cell based immuno-fluorescence assay using the MCF-7 human ductal carcinoma breast cell line. MCF-7 cells were revived directly from a cryovial (approx 5×10⁶ cells) in Assay Medium (phenol red free Dulbecco's Modified Eagle's medium (DMEM); Sigma D5921) containing 2 mM L-Glutamine and 5% (v/v) Charcoal/Dextran treated foetal calf serum. Cells were syringed once using a sterile 18G×1.5 inch (1.2×40 mm) broad gauge needle and cell density was measured using a Coulter Counter (Beckman). Cells were further diluted in Assay Medium to a density of 3.75×10⁴ cells per mL and 40 μL per well added to transparent bottomed, black, tissue culture-treated 384 well plates (Costar, No. 3712) using a Thermo Scientific Matrix WellMate or Thermo Multidrop. Following cell seeding, plates were incubated overnight at 37° C., 5% CO₂ (Liconic carousel incubator). Test data was generated using the LabCyte Echo™ model 555 compound reformatter which is part of an automated workcell (Integrated Echo 2 workcell). Compound stock solutions (10 mM) of the test compounds were used to generate a 384 well compound dosing plate (Labcyte P-05525-CV1). 40 μL of each of the 10 mM compound stock solutions was dispensed into the first quadrant well and then 1:100 step-wise serial dilutions in DMSO were performed using a Hydra II (MATRIX UK) liquid handling unit to give 40 μL of diluted compound into quadrant wells 2 (0.1 mM), 3 (1 μM) and 4 (0.01 μM), respectively. 40 μL of DMSO added to wells in row P on the source plate allowed for DMSO normalisation across the dose range. To dose the control wells 40 μL of DMSO was added to row O1 and 40 μL of 100 μM fulvestrant in DMSO was added to row 03 on the compound source plate.

The Echo uses acoustic technology to perform direct microplate-to-microplate transfers of DMSO compound solutions to assay plates. The system can be programmed to transfer volumes as low as 2.5 nL in multiple increments between microplates and in so doing generates a serial dilution of compound in the assay plate which is then back-filled to normalise the DMSO concentration across the dilution range. Compounds were dispensed onto the cell plates with a compound source plate prepared as above producing a 12 point duplicate 3 μM to 3 μM dose range with 3-fold dilutions and one final 10-fold dilution using the Integrated Echo 2 workcell. The maximum signal control wells were dosed with DMSO to give a final concentration of 0.3%, and the minimum signal control wells were dosed with fulvestrant to give a final concentration of 100 nM accordingly. Plates were further incubated for 18-22 hours at 37° C., 5% CO₂ and then fixed by the addition of 20 μL of 11.1% (v/v) formaldehyde solution (in phosphate buffered saline (PBS)) giving a final formaldehyde concentration of 3.7% (v/v). Cells were fixed at room temperature for 20 mins before being washed two times with 250 μL PBS/Proclin (PBS with a Biocide preservative) using a BioTek platewasher, 40 μL of PBS/Proclin was then added to all wells and the plates stored at 4° C. The fixing method described above was carried out on the Integrated Echo 2 workcell. Immunostaining was performed using an automated AutoElisa workcell. The PBS/Proclin was aspirated from all wells and the cells permeabilised with 40 μL PBS containing 0.5% Tween™ 20 (v/v) for 1 hour at room temperature. The plates were washed three times in 250 μL of PBS/0.05% (v/v) Tween 20 with Proclin (PBST with a Biocide preservative) and then 20 μL of ERα (SPI) Rabbit monoclonal antibody (Thermofisher) 1:1000 in PBS/Tween™/3% (w/v) Bovine Serum Albumin was added. The plates were incubated overnight at 4° C. (Liconic carousel incubator) and then washed three times in 250 μL of PBS/0.05% (v/v) Tween™ 20 with Proclin (PBST). The plates were then incubated with 20 μL/well of a goat anti-rabbit IgG AlexaFluor 594 antibody with Hoechst at 1:5000 in PBS/Tween™/3% (w/v) Bovine Serum Albumin for 1 hour at room temperature. The plates were then washed three times in 250 μL of PBS/0.05% (v/v) Tween™ 20 with Proclin (PBST with a Biocide preservative). 20 μL of PBS was added to each well and the plates covered with a black plate seal and stored at 4° C. before being read.

Plates were read using a Cellomics Cellinsight reading the 594 nm to measure the ERα receptor level in each well. The MEAN_CircSpotTotalInten algorithm was calculated used to represent ERα expression. The data was exported into Genedata to perform curve fitting analysis. Down-regulation of the ERα receptor was expressed as an IC₅₀ value and was determined by calculation of the concentration of compound that was required to give a 50% reduction of ERα expression.

The data shown in Table A were generated (the data below may be a result from a single experiment or an average of two or more experiments).

TABLE A

| | | MCF-7 ER |
|---|---|---|
| | ER binding IC50 | degradation |
| Example | (nM) | IC50 (nM) |
| 1 | 3.1 | 1.1 |
| 2 | 3.3 | 0.5 |
| 3 | 3.7 | 6.3 |
| 4 | 2.1 | 0.5 |
| 5 | 3.7 | 0.6 |
| 6 | 5.6 | 0.6 |
| 7 | 4.7 | g0.4 |
| 8 | 4.3 | 1.0 |
| 9 | 8.7 | 0.6 |
| 10 | 22.3 | 0.8 |
| 11 | 1.2 | 0.4 |
| 12 | 1.5 | 0.4 |
| 13 | 3.0 | 0.6 |
| 14 | 8.5 | 0.9 |
| 15 | 3.7 | 0.6 |
| 16 | 3.0 | 0.6 |
| 17 | 13.8 | 0.8 |
| 18 | 9.5 | 0.8 |
| 19 | 5.3 | 0.4 |
| 20 | 2.0 | 0.5 |
| 21 | 7.6 | 0.9 |
| 22 | 2.1 | 0.7 |
| 23 | 4.4 | 0.5 |
| 24 | 6.8 | 1.1 |
| 25 | 2.6 | 0.6 |
| 26 | 18.0 | 1.1 |
| 27 | 2.3 | 0.7 |
| 28 | 1.9 | 0.4 |
| 29 | 12.3 | 1.4 |
| 30 | 5.4 | 0.7 |
| 31 | 5.1 | 0.7 |
| 32 | 3.3 | 1.2 |
| 33 | 3.9 | 1.0 |
| 34 | 7.3 | 1.0 |
| 35 | 6.9 | 1.7 |
| 36 | 3.2 | 1.7 |
| 37 | 3.3 | 0.4 |
| 38 | 3.6 | 0.4 |
| 39 | 3.2 | 0.7 |
| 40 | 7.0 | 0.8 |
| 41 | 2.2 | 0.4 |

According to a further aspect of the specification there is provided a pharmaceutical composition, which comprises a compound of the Formula (I) or a pharmaceutically acceptable salt thereof, as defined hereinbefore in association with a pharmaceutically acceptable excipient.

The compositions may be in a form suitable for oral use (for example as tablets, lozenges, hard or soft capsules, aqueous or oily suspensions, emulsions, dispersible powders or granules, syrups or elixirs) or for parenteral administration (for example as a sterile aqueous or oily solution for intravenous, subcutaneous or intramuscular dosing). The compositions may be obtained by conventional procedures using conventional pharmaceutical excipients, well known in the art. Thus, compositions intended for oral use may contain, for example, one or more colouring, sweetening, flavouring and/or preservative agents.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration.

The size of the dose for therapeutic or prophylactic purposes of compounds of the present specification will naturally vary according to the nature and severity of the disease state, the age and sex of the animal or patient and the route of administration, according to well known principles of medicine.

As stated above, it is known that signalling through ERα causes tumourigenesis by one or more of the effects of mediating proliferation of cancer and other cells, mediating angiogenic events and mediating the motility, migration and invasiveness of cancer cells. We have found that the compounds of the present specification possess potent anti-proliferative activity in ER positive breast cancer cell lines which is believed to be a result of antagonism and degradation of ERα protein.

Accordingly, the compounds of the present specification may be of value as anti-tumour agents, in particular as selective inhibitors of the proliferation, survival, motility, dissemination and invasiveness of mammalian cancer cells leading to inhibition of tumour growth and survival and to inhibition of metastatic tumour growth. Particularly, the compounds of the present specification may be of value as anti-proliferative and anti-invasive agents in the containment and/or treatment of solid tumour disease. Particularly, the compounds of the present specification may be useful in the prevention or treatment of those tumours which are sensitive to inhibition of ERα and that are involved in the signal transduction steps which lead to the proliferation and survival of tumour cells and the migratory ability and invasiveness of metastasising tumour cells. Further, the compounds of the present specification may be useful in the prevention or treatment of those tumours which are mediated alone or in part by antagonism and degradation of ERα, i.e. the compounds may be used to produce an ERα inhibitory effect in a warm-blooded animal in need of such treatment.

According to a further aspect of the specification there is provided a compound of the Formula (I) or a pharmaceutically acceptable salt thereof, as defined hereinbefore for use as a medicament in a warm-blooded animal such as man.

According to a further aspect of the specification, there is provided a compound of the Formula (I), or a pharmaceutically acceptable salt thereof, as defined hereinbefore for use in the production of an anti-proliferative effect in a warm-blooded animal such as man.

According to a further aspect of the specification there is provided the use of a compound of the Formula (I), or a pharmaceutically acceptable salt thereof, as defined hereinbefore, in the manufacture of a medicament for use in the production of an anti-proliferative effect in a warm-blooded animal such as man.

According to a further aspect of the specification there is provided a method for producing an anti-proliferative effect in a warm-blooded animal, such as man, in need of such treatment which comprises administering to said animal an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore for use in a warm-blooded animal such as man as an anti-invasive agent in the containment and/or treatment of solid tumour disease.

According to a further aspect of the specification there is provided the use of a compound of the Formula (I), or a pharmaceutically acceptable salt thereof, as defined hereinbefore, in the manufacture of a medicament for use in a warm-blooded animal such as man as an anti-invasive agent in the containment and/or treatment of solid tumour disease.

According to a further aspect of the specification there is provided a method for producing an anti-invasive effect by the containment and/or treatment of solid tumour disease in a warm-blooded animal, such as man, in need of such treatment which comprises administering to said animal an effective amount of a compound of the Formula (I), or a pharmaceutically acceptable salt thereof, as defined hereinbefore.

According to a further aspect of the specification, there is provided a compound of the Formula (I), or a pharmaceutically acceptable salt thereof, as defined hereinbefore, for use in the prevention or treatment of cancer in a warm-blooded animal such as man.

According to a further aspect of the specification there is provided the use of a compound of the Formula (I), or a pharmaceutically acceptable salt thereof, as defined hereinbefore in the manufacture of a medicament for use in the prevention or treatment of cancer in a warm-blooded animal such as man.

According to a further aspect of the specification there is provided a method for the prevention or treatment of cancer in a warm-blooded animal, such as man, in need of such treatment which comprises administering to said animal an effective amount of a compound of the Formula (I), or a pharmaceutically acceptable salt thereof, as defined hereinbefore.

According to a further aspect of the specification, there is provided a compound of the Formula (I), or a pharmaceutically acceptable salt thereof, as defined hereinbefore for use in the prevention or treatment of solid tumour disease in a warm-blooded animal such as man.

According to a further aspect of the specification there is provided the use of a compound of the Formula (I), or a pharmaceutically acceptable salt thereof, as defined hereinbefore, in the manufacture of a medicament for use in the prevention or treatment of solid tumour disease in a warm-blooded animal such as man.

According to a further aspect of the specification there is provided a method for the prevention or treatment of solid tumour disease in a warm-blooded animal, such as man, in need of such treatment which comprises administering to said animal an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore, for use in the prevention or treatment of those tumours which are sensitive to inhibition of ERα that are involved in the signal transduction steps which lead to the proliferation, survival, invasiveness and migratory ability of tumour cells.

According to a further aspect of the specification there is provided the use of a compound of the Formula (I), or a pharmaceutically acceptable salt thereof, as defined hereinbefore, in the manufacture of a medicament for use in the prevention or treatment of those tumours which are sensitive to inhibition of ERα that are involved in the signal transduction steps which lead to the proliferation, survival, invasiveness and migratory ability of tumour cells.

According to a further aspect of the specification there is provided a method for the prevention or treatment of those tumours which are sensitive to inhibition of ERα that are involved in the signal transduction steps which lead to the proliferation, survival, invasiveness and migratory ability of tumour cells which comprises administering to said animal an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore for use in providing an inhibitory effect on ERα.

According to a further aspect of the specification there is provided the use of a compound of the Formula (I), or a pharmaceutically acceptable salt thereof, as defined hereinbefore in the manufacture of a medicament for use in providing an inhibitory effect on ERα.

According to a further aspect of the specification there is also provided a method for providing an inhibitory effect on ERα which comprises administering an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore, for use in providing a selective inhibitory effect on ERα.

According to a further aspect of the specification there is provided the use of a compound of the Formula (I), or a pharmaceutically acceptable salt thereof, as defined hereinbefore, in the manufacture of a medicament for use in providing a selective inhibitory effect on ERα.

According to a further aspect of the specification there is also provided a method for providing a selective inhibitory effect on ERα which comprises administering an effective amount of a compound of the Formula (I), or a pharmaceutically acceptable salt thereof, as defined hereinbefore.

Described herein are compounds that can bind to ERα ligand binding domain and selectively induce ERα degradation. In biochemical and cell based assays the compounds of the present specification are shown to be potent estrogen receptor binders and reduce cellular levels of ERα and may therefore be useful in the treatment of estrogen sensitive diseases or conditions (including diseases that have developed resistance to endocrine therapies), i.e. for use in the treatment of cancer of the breast and gynaecological cancers (including endometrial, ovarian and cervical) and cancers expressing ERα mutated proteins which may be de novo mutations or have arisen as a result of treatment with a prior endocrine therapy such as an aromatase inhibitor.

According to a further aspect of the specification there is provided a compound of the Formula (I), or a pharmaceutically acceptable salt thereof, as defined hereinbefore, for use in the treatment of breast or gynaecological cancers.

According to a further aspect of the specification there is provided the use of a compound of the Formula (I), or a pharmaceutically acceptable salt thereof, as defined hereinbefore, in the manufacture of a medicament for use in the treatment of breast or gynaecological cancers.

According to a further aspect of the specification there is provided a method for treating breast or gynaecological cancers, which comprises administering an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore, for use in the treatment of cancer of the breast, endometrium, ovary or cervix.

According to a further aspect of the specification there is provided the use of a compound of the Formula (I), or a pharmaceutically acceptable salt thereof, as defined hereinbefore, in the manufacture of a medicament for use in the treatment of cancer of the breast, endometrium, ovary or cervix.

According to a further aspect of the specification there is provided a method for treating cancer of the breast, endometrium, ovary or cervix, which comprises administering an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore, for use in the treatment of breast cancer.

According to a further aspect of the specification there is provided the use of a compound of the Formula (I), or a pharmaceutically acceptable salt thereof, as defined hereinbefore, in the manufacture of a medicament for use in the treatment of breast cancer.

According to a further aspect of the specification there is provided a method for treating breast cancer, which comprises administering an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore, for use in the treatment of breast cancer, wherein the cancer has developed resistance to one or more other endocrine therapies.

According to a further aspect of the specification there is provided the use of a compound of the Formula (I), or a pharmaceutically acceptable salt thereof, as defined hereinbefore, in the manufacture of a medicament for use in the treatment of breast cancer, wherein the cancer has developed resistance to one or more other endocrine therapies.

According to a further aspect of the specification there is provided a method for treating breast cancer, wherein the cancer has developed resistance to one or more other endocrine therapies, which comprises administering an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore, for use in the treatment of ER+ve breast cancer.

According to a further aspect of the specification there is provided the use of a compound of the Formula (I), or a pharmaceutically acceptable salt thereof, as defined herein before in the manufacture of a medicament for use in the treatment of ER+ve breast cancer.

According to a further aspect of the specification there is provided a method for treating ER+ve breast cancer, which comprises administering an effective amount of a compound of the Formula (I), or a pharmaceutically acceptable salt thereof, as defined hereinbefore.

The anti-cancer treatment defined herein may be applied as a sole therapy or may involve, in addition to the compounds of the specification, conventional surgery or radiotherapy or chemotherapy. Such chemotherapy may include the following category of anti-tumour agents:—

(i) inhibitors of CDK4/6 such as palbociclib, ribociclib and abemaciclib.

In one aspect the above combinations, pharmaceutical compositions, uses and methods of treating cancer, are methods for the treatment of breast or gynaecological cancers, such as cancer of the breast, endometrium, ovary or cervix, particularly breast cancer, such as ER+ve breast cancer.

According to a further aspect of the present specification there is provided a kit comprising a compound of Formula (I), or a pharmaceutically acceptable salt thereof in combination with an anti-tumour agent selected from one listed above.

Combination therapy as described above may be added on top of standard of care therapy typically carried out according to its usual prescribing schedule.

Although the compounds of the Formula (I) are primarily of value as therapeutic agents for use in warm-blooded animals (including man), they are also useful whenever it is required to inhibit ER-α. Thus, they are useful as pharmacological standards for use in the development of new biological tests and in the search for new pharmacological agents.

### EXAMPLES

The disclosure will now be further explained by reference to the following illustrative examples.

Unless stated otherwise, starting materials were commercially available. All solvents and commercial reagents were of laboratory grade and were used as received.

### General Experimental

The disclosure will now be illustrated in the following Examples in which, generally:
(i) operations were carried out at room temperature (RT), i.e. in the range 17 to 25° C. and under an atmosphere of an inert gas such as N₂ or Ar unless otherwise stated;
(ii) in general, the course of reactions was followed by thin layer chromatography (TμC) and/or analytical high-performance liquid chromatography (HPLC or UPLC) which was usually coupled to a mass spectrometer (LCMS).
The reaction times that are given are not necessarily the minimum attainable;
(iii) when necessary, organic solutions were dried over anhydrous MgSO₄ or Na₂SO₄, work-up procedures were carried out using traditional phase separating techniques or by using SCX as described in (xiii), evaporations were carried out either by rotary evaporation in vacuo or in a Genevac HT-4/EZ-2 or Biotage V10;
(iv) yields, where present, are not necessarily the maximum attainable, and when necessary, reactions were repeated if a larger amount of the reaction product was required;
(v) in general, the structures of the end-products of the Formula (I) were confirmed by nuclear magnetic resonance (NMR) and/or mass spectral techniques; electrospray mass spectral data were obtained using a Waters Acquity UPLC coupled to a Waters single quadrupole mass spectrometer acquiring both positive and negative ion data, and generally, only ions relating to the parent structure are reported, the error inherent to the instrument is ±0.3 Da and masses were recorded as observed; proton NMR chemical shift values were measured on the delta scale using either a Bruker AV500 spectrometer operating at a field strength of 500 MHz, a Bruker AV400 operating at 400 MHz or a Bruker AV300 operating at 300 MHz. Unless otherwise stated, NMR spectra were obtained at 500 MHz in d6-dimethylsulfoxide. The following abbreviations have been used: s, singlet; d, doublet; t, triplet; q, quartet; m, multiplet; br, broad; qn, quintet; electrospray high resolution mass spectrometry data were obtained using a Waters Acquity UPLC coupled to a Bruker micrOTOF-Q II quadrupole time-of-flight mass spectrometer acquiring positive ion data or equivalent;
(vi) Unless stated otherwise compounds containing an asymmetric carbon and/or sulfur atom were not resolved;
(vii) Intermediates were not necessarily fully purified but their structures and purity were assessed by TLC, analytical HPLC/UPLC, and/or NMR analysis and/or mass spectrometry;
(viii) unless otherwise stated, flash column chromatography was performed on Merck Kieselgel silica (Art. 9385) or on reversed phase silica (Fluka silica gel 90 C18) or on Silicycle cartridges (40-63 μm silica, 4 to 330 g weight) or on Grace resolv cartridges (4-120 g) or on RediSep Rf 1.5 Flash columns or on RediSep Rf high performance Gold Flash columns (150-415 g weight) or on RediSep Rf Gold C18 Reversed-phase columns (20-40 μm silica) either manually or automated using a Teledyne Isco CombiFlash Companion, Teledyne Isco Combiflash Rf or Teledyne Isco Rf Lumen system or similar system;
(ix) Preparative reverse phase HPLC (RP HPLC) was performed on C18 reversed-phase silica typically using a Waters XSelect CSH C18 OBD column (5 μm silica, 30 mm diameter, 100 mm length) using decreasingly polar mixtures as eluent, for example utilising water as solvent A and acetonitrile as solvent B [with additional modifier stream to provide a mobile phase containing 0.1-5% formic acid or 0.1-5% aqueous ammonium hydroxide (d=0.91)]; a typical procedure would be as follows: a solvent gradient over 10-20 minutes, at 40-50 mL per minute, from a 95:5 mixture of solvents A and B respectively to a 5:95 mixture of solvents A and B (or alternative ratio as appropriate).
(x) The following analytical UPLC methods were used; in general, reverse-phase C18 silica was used with a flow rate of 1 mL/minute and detection was by Electrospray Mass Spectrometry and by UV absorbance recording a wavelength range of 220-320 nm. Analytical UPLC was performed on CSH C18 reverse-phase silica, using a Waters XSelect CSH C18 column with dimensions 2.1×50 mm and particle size 1.7 micron). Gradient analysis was employed using decreasingly polar mixtures as eluent, for example decreasingly polar mixtures of water (containing 0.1% formic acid or 0.1% ammonia) as solvent A and acetonitrile as solvent B. A typical 2 minute analytical UPLC method would employ a solvent gradient over 1.3 minutes, at approximately 1 mL per minute, from a 97:3 mixture of solvents A and B respectively to a 3:97 mixture of solvents A and B.
(xi) Where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;
(xii) Where reactions refer to the use of a microwave, one of the following microwave reactors were used: Biotage Initiator, Personal Chemistry Emrys Optimizer, Personal Chemistry Smithcreator or CEM Explorer;
(xiii) Compounds were purified by strong cation exchange (SCX) chromatography using Isolute SPE flash SCX-2 or SCX-3 columns (International Sorbent Technology Limited, Mid Glamorgan, UK);
(xiv) the following preparative chiral HPLC methods were carried out using a Gilson GX-281 HPLC and a DAICEL CHIRALPAK IC (2×25 cm, 5 um) or DAICEL CHIRALPAK IF (2×25 cm, 5 um); in general a flow rate of between 10-350 mL/minute and detection was by UV absorbance at a typical wavelength of 254 nm. A sample concentration of about 1-100 mg/mL was used in a suitable solvent mixture with an injection volume of between 0.5-10 mL and run time of between 10-150 minutes and a typical oven temperature of 25-35° C.; (xv) the following analytical chiral HPLC methods were carried out using Shimadzu UFLC and a Daicel CHIRALPAK IC-3 (50×4.6 mm 3 um) or Daicel CHIRALPAK IF-3 (50×4.6 mm 3 um); in general a flow rate of 1 mL/minute and detection was by UV absorbance at a typical wavelength of 254 nm. A sample concentration of about 1 mg/mL was used in a suitable solvent such as EtOH with an injection volume of about 10 μL and run time of between 10-60 minutes and a typical oven temperature of 25-35° C.;
(xvi) the following preparative chiral supercritical fluid chromatography (SFC) methods were used; in general a flow rate of about 70 mL/minute and detection was by UV absorbance at a typical wavelength of 254 nm. A sample concentration of about 100 mg/mL was used in a suitable solvent such as MeOH with an injection volume of about 0.5 mL and run time of between 10-150 minutes and a typical oven temperature of 25-35° C.;
(xvii) in general Examples and intermediate compounds were named using ACD Name, “Structure to Name” part of ChemDraw Ultra (CambridgeSoft) or Biovia Draw 2016;
(xviii) In addition to the ones mentioned above, the following abbreviations have been used:

| | | | |
|---|---|---|---|
| AcOH | acetic acid | aq. | Aqueous |
| DCM | dichloromethane | DIPEA | N,N-diisopropylethylamine |
| Boc | tert-butyloxycarbonyl | BPR | back pressure regulator |
| Cbz | carboxybenzyl | CDCl₃ | deuterated chloroform |
| DIAD | diisopropyl azodicarboxylate | DEA | diethanolamine |
| DMF | N,N-dimethylformamide | DMSO | Dimethyl sulfoxide |
| eq. | equivalents | ESI-HRMS | electrospray ionisation - high |
| | | | resolution mass spectrometry |
| Et₂O | diethyl ether | EtOAc | ethyl acetate |
| EtOH | ethanol | HATU | 2-(3H-[1,2,3]Triazolo[4,5- |
| | | | b]pyridin-3-y1)-1,1,3,3- |
| | | | tetramethylisouronium |
| | | | hexafluorophosphate(V) |
| HPLC | high-performance liquid | IPA | isopropyl alcohol |
| | chromatography | | |
| MeCN | acetonitrile | MeOD | d₄-methanol |
| MeOH | methanol | m/z | mass spectrometry peak(s) |
| MgSO₄ | magnesium sulfate | NaHCO₃ | sodium bicarbonate |
| NH₄OH | ammonium hydroxide | Pd-PEPPSI- | dichloro[1,3-bis(2,6-di-4- |
| | | IHept^{Cl} | heptylphenypimidazol-2- |
| | | | yldiene(3- |
| | | | chloropyridyl)palladium(II) |
| RockPhos | [(2-Di-tert-butylphosphino-3- | RT | room temperature |
| Pd G3 | methoxy-6-methy1-2′,4′,6′- | | |
| | triisopropyl-1,1′-biphenyl)-2-(2- | | |
| | aminobiphenyl)]palladium(II) | | |
| | methanesulfonate | | |
| TBAF | tetra n-butylammonium fluoride | THF | tetrahydrofuran |
| Sat. | saturated | scCO₂ | Supercritical carbon dioxide |
| SCX | Strong cation exchange | SFC | Supercritical fluid |
| | | | chromatography |

### Intermediate 1a: (1R,3R)-1-(2-Chloropyrimidin-5-yl)-2-(2-fluoro-2-methylpropyl)-3-methyl-2,3,4,9-tetrahydro-1H-pyrido[3,4-b]indole

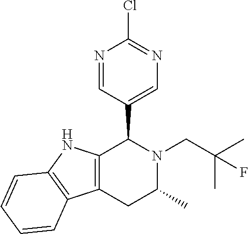 [SMILES: C[C@@H]1Cc2c([nH]c3ccccc23)[C@@H](c2cnc(Cl)nc2)N1CC(C)(C)F]

A solution of (R)—N-(1-(1H-indol-3-yl)propan-2-yl)-2-fluoro-2-methylpropan-1-amine (14.56 g, 58.65 mmol) and 2-chloropyrimidine-5-carbaldehyde (8.36 g, 58.7 mmol) in toluene (285 mL) and acetic acid (29 mL) was stirred at 90° C. for 4 h. The reaction mixture was allowed to cool to RT, concentrated and diluted with DCM (250 mL), and washed with sat. NaHCO₃ (2×200 mL) and sat. brine (150 mL). The organic layer was dried with a phase separating cartridge, filtered and evaporated. The crude product was purified by flash silica chromatography, elution gradient 0 to 50% EtOAc in heptane to afford the title compound (16.0 g, 73%) as a cream solid; ¹H NMR (400 MHz, CDCl₃, 30° C.) 1.12 (31H, d), 1.31 (31H, d), 1.51 (31H, d), 2.5-2.64 (21H, in), 2.64-2.77 (21H, in), 3.05 (1H, ddd), 5.22 (1H, d), 7.15 (1H, td), 7.22 (1H, td), 7.34 (1H, d), 7.55 (1H, d), 7.79 (1H, s), 8.53 (2H, d); m/z: ES+ [M+H]⁺ 373.0.

### Intermediate 1b: Benzyl 4-(dimethoxymethyl)piperidine-1-carboxylate

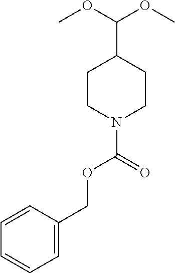 [SMILES: COC(OC)C1CCN(C(=O)OCc2ccccc2)CC1]

4-Formyl-N-Cbz-piperidine (5.00 g, 20.22 mmol) was dissolved in MeOH (11.4 mL) at 0° C. under N₂ and a solution of titanium(IV) chloride (0.11 mL, 1.01 mmol) in DCM (1.1 mL) was then added and after 15 minutes triethylamine (0.338 mL, 2.43 mmol). The resulting solution was stirred at 20° C. for 30 minutes. The reaction mixture was diluted with DCM (50 mL) and water (20 mL) and stirred at RT for 30 minutes. The layers were separated, the organic layer dried over a hydrophobic frit and concentrated. The product was purified by flash silica chromatography, elution gradient 0 to 50% EtOAc in heptane to afford the title compound (5.16 g, 87%) as a colourless oil; ¹H NMR (400 MHz, CDCl₃, 30° C.) 1.14-1.33 (2H, m), 1.63-1.82 (3H, m), 2.63-2.84 (2H, m), 3.35 (6H, s), 4.02 (1H, d), 4.13-4.3 (2H, m), 5.12 (2H, s), 7.3-7.44 (5H, m).

### Intermediate 1c: 4-(Dimethoxymethyl)piperidine

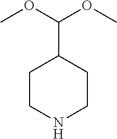

Dihydroxypalladium 10 wt % (0.73 g, 0.52 mmol) was added to benzyl 4-(dimethoxymethyl)piperidine-1-carboxylate (7.60 g, 25.9 mmol) in MeOH (60 mL) at 20° C. under N₂ in a steel pressured reactor. The resulting suspension was purged with N₂ and H₂ and stirred at 20° C. at 4 atm for 2 days. The reaction mixture was filtered over celite and washed with MeOH (500 mL). The filtrate was concentrated to afford the title compound (4.0 g, 97%) as a colorless oil; ¹H NMR (400 MHz, CDCl₃, 30° C.) 1.19-1.41 (2H, m), 1.69-1.86 (3H, m), 2.61 (2H, td), 3.15 (2H, d), 3.35 (6H, s), 4.03 (1H, d), 4.47 (1H, s).

### Intermediate 1d: (1R,3R)-1-(2-(4-(Dimethoxymethyl)piperidin-1-yl)pyrimidin-5-yl)-2-(2-fluoro-2-methylpropyl)-3-methyl-2,3,4,9-tetrahydro-1H-pyrido[3,4-b]indole

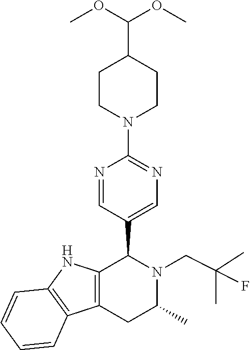 [SMILES: COC(OC)C1CCN(c2ncc([C@@H]3c4[nH]c5ccccc5c4C[C@@H](C)N3CC(C)(C)F)cn2)CC1]

(1R,3R)-1-(2-Chloropyrimidin-5-yl)-2-(2-fluoro-2-methylpropyl)-3-methyl-2,3,4,9-tetrahydro-1H-pyrido[3,4-b]indole (250 mg, 0.67 mmol), 4-(dimethoxymethyl)piperidine (107 mg, 0.67 mmol) and DIPEA (0.35 mL, 2.01 mmol) were stirred in DMF (5 mL) at 90° C. for 4 h. The reaction mixture was cooled to RT and diluted with EtOAc (25 mL) and water (25 mL). The organics were separated and washed with brine (25 mL), dried over anhydrous Na₂SO₄, filtered and concentrated under reduced pressure. The crude product was purified by flash silica chromatography, elution gradient 0 to 100% EtOAc in heptane to afford the title compound (268 mg, 81%) as a pale yellow oil; ¹H NMR (400 MHz, CDCl₃, 30° C.) 1.09 (3H, d), 1.31 (3H, d), 1.46 (3H, d), 1.55 (2H, d), 1.81 (2H, d), 1.88 (1H, ddd), 2.48-2.61 (2H, m), 2.67 (2H, d), 2.76-2.86 (2H, m), 3.24 (1H, d), 3.36 (6H, s), 4.03 (1H, d), 4.76 (2H, d), 4.98 (1H, s), 7.06-7.19 (2H, m), 7.27 (1H, d), 7.51 (1H, d), 7.73 (1H, d), 8.17 (2H, d); m/z: ES+ [M+H]⁺ 496.4.

### Intermediate 1e: 1-(5-((1R,3R)-2-(2-Fluoro-2-methylpropyl)-3-methyl-2,3,4,9-tetrahydro-1H-pyrido[3,4-b]indol-1-yl)pyrimidin-2-yl)piperidine-4-carbaldehyde

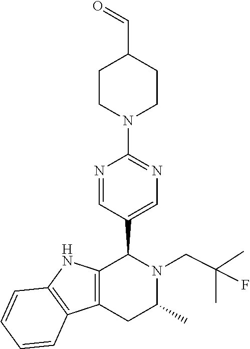 [SMILES: C[C@@H]1Cc2c([nH]c3ccccc23)[C@@H](c2cnc(N3CCC(C=O)CC3)nc2)N1CC(C)(C)F]

Sulfuric acid (2M) (2.70 mL, 5.41 mmol) was added dropwise to (1R,3R)-1-(2-(4-(dimethoxymethyl)piperidin-1-yl)pyrimidin-5-yl)-2-(2-fluoro-2-methylpropyl)-3-methyl-2,3,4,9-tetrahydro-1H-pyrido[3,4-b]indole (134 mg, 0.27 mmol) in THF (5 mL) at RT. The solution was stirred for 20 mins at RT then diluted with water (20 mL) and EtOAc (20 mL). The organics were separated and the aqueous neutralised with NaHCO₃ solution (pH 7-8) and extracted with EtOAc (2×20 mL). The combined organics were washed with sat. NaCl solution, dried over anhydrous Na₂SO₄, filtered and concentrated under vacuum to afford the title compound as a yellow oil (used directly in the next step without purification); m/z: ES+ [M+H]⁺ 450.4.

### Intermediate 1f: tert-Butyl 4-(1-oxo-1,3-dihydroisobenzofuran-5-yl)piperazine-1-carboxylate

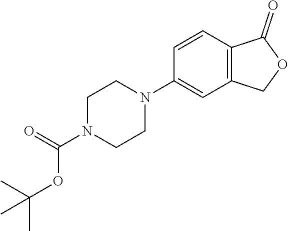 [SMILES: CC(C)(C)OC(=O)N1CCN(c2ccc3c(c2)COC3=O)CC1]

To a solution of 5-bromoisobenzofuran-1(3H)-one (9.0 g, 42.3 mmol) and tert-butyl piperazine-1-carboxylate (7.87 g, 42.3 mmol) in 1,4-dioxane (100 mL) was added Pd₂(dba)₃ (3.87 g, 4.22 mmol) and (9,9-dimethyl-9H-xanthene-4,5-diyl)bis(diphenylphosphane) (2.45 g, 4.22 mmol) and potassium phosphate (17.94 g, 84.50 mmol). The mixture was stirred at 100° C. for 18 h under N₂. The mixture was cooled to RT and filtered through a pad of celite, washed with EtOAc (100 mL). The filtrate was concentrated under reduced pressure. The residue was triturated in EtOAc:heptane (100 mL, v/v=1:1), filtered, washed with Et₂O (200 mL) and dried to afford the title compound (10.6 g, 79%) as an orange solid; ¹H NMR (400 MHz, CDCl₃, 30° C.) 1.49 (9H, s), 3.31-3.42 (4H, m), 3.55-3.67 (4H, m), 5.21 (2H, s), 6.80 (1H, s), 6.98 (1H, dd), 7.76 (1H, d); m/z: ES+ [M+H]⁺ 319.3.

### Intermediate 12: 4-(4-(tert-Butoxycarbonyl)piperazin-1-yl)-2-(hydroxymethyl)benzoic acid

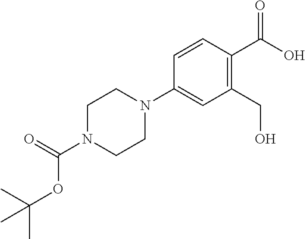 [SMILES: CC(C)(C)OC(=O)N1CCN(c2ccc(C(=O)O)c(CO)c2)CC1]

Sodium hydroxide (5.33 g, 133.2 mmol) was added portionwise to a solution of tert-butyl 4-(1-oxo-1,3-dihydroisobenzofuran-5-yl)piperazine-1-carboxylate (10.6 g, 33.3 mmol) in MeOH (25 mL), THF (25 mL) and water (25 mL) and stirred at RT for 1 h. The solution was adjusted to pH4-5 with HCl (2M) and extracted into EtOAc (250 mL×3). The organic layers were washed with brine (100 mL), dried over anhydrous Na₂SO₄ and concentrated under reduced pressure. The crude material was triturated with Et₂O (100 mL) and collected by vacuum filtration to afford the title compound (8.23 g, 74%) as a yellow solid; ¹H NMR (400 MHz, DMSO, 30° C.) 1.43 (9H, s), 3.28 (4H, s), 3.41-3.57 (4H, m), 4.80 (2H, s), 5.08 (1H, s), 6.82 (1H, dd), 7.22 (1H, d), 7.79 (1H, d), 12.24 (1H, s); m/z: ES+ [M+H]⁺ 337.0

### Intermediate 1h: tert-Butyl 4-(3-(hydroxymethyl)-4-(methoxycarbonyl)phenyl)piperazine-1-carboxylate

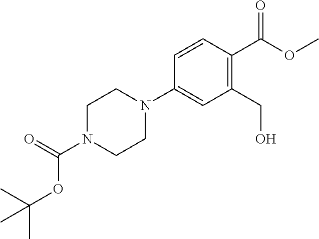 [SMILES: COC(=O)c1ccc(N2CCN(C(=O)OC(C)(C)C)CC2)cc1CO]

To a solution of 4-(4-(tert-butoxycarbonyl)piperazin-1-yl)-2-(hydroxymethyl)benzoic acid (3.25 g, 9.66 mmol) in MeOH (20 mL) and EtOAc (20 mL) at −10° C., was added TMS-diazomethane (2M in hexane, 14.5 mL, 30.0 mmol) dropwise. The solution was stirred at −10° C. for 1 h and then diluted with water (100 mL) and extracted with EtOAc (100 mL×3). The organics were dried over anhydrous Na₂SO₄, filtered and concentrated under reduced pressure to afford the title compound as an oil (assumed quant); m/z: ES+ [M+H]⁺ 351.0

### Intermediate 1i: tert-Butyl 4-(3-(bromomethyl)-4-(methoxycarbonyl)phenyl)piperazine-1-carboxylate

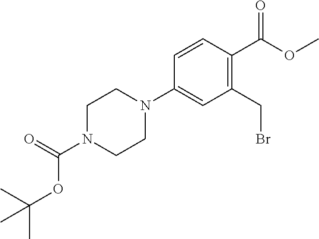 [SMILES: COC(=O)c1ccc(N2CCN(C(=O)OC(C)(C)C)CC2)cc1CBr]

To a solution of tert-butyl 4-(3-(hydroxymethyl)-4-(methoxycarbonyl)phenyl)piperazine-1-carboxylate (3.39 g, 9.66 mmol) in THF (10 mL) was added triphenylphosphine (3.80 g, 14.5 mmol) and perbromomethane (4.81 g, 14.5 mmol). The solution was stirred at 25° C. for 1 h, quenched with water (200 mL) and extracted with EtOAc (100 mL×2). The organic layer was dried over Na₂SO₄, filtered and concentrated under vacuum. The product was purified by flash silica chromatography, elution gradient 0 to 50% EtOAc in heptane to afford the title compound (2.2 g, 55%) as a white solid; ¹H NMR (400 MHz, CDCl₃, 30° C.) 1.49 (9H, s), 3.24-3.38 (4H, m), 3.54-3.62 (4H, m), 3.89 (3H, s), 4.96 (2H, s), 6.78 (1H, dd), 6.88 (1H, d), 7.93 (1H, d).

### Intermediate 1j: tert-Butyl-4-(2-(2,6-dioxopiperidin-3-yl)-1-oxoisoindolin-5-yl)piperazine-1-carboxylate

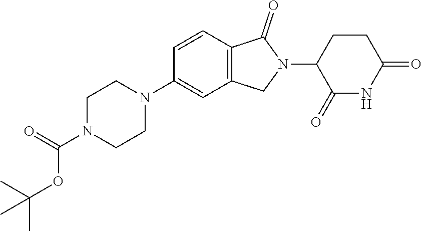 [SMILES: CC(C)(C)OC(=O)N1CCN(c2ccc3c(c2)CN(C2CCC(=O)NC2=O)C3=O)CC1]

To a solution of tert-butyl 4-(3-(bromomethyl)-4-(methoxycarbonyl)phenyl)piperazine-1-carboxylate (2.20 g, 5.32 mmol) in MeCN (30 mL) was added 3-aminopiperidine-2,6-dione, HCl (1.31 g, 7.98 mmol) and DIPEA (2.8 mL, 16.0 mmol). The solution was stirred at 80° C. for 4 h then stirred at RT for 72 h. The reaction mixture was warmed to 80° C. for 24 h. The reaction mixture was cooled to RT and concentrated under reduced pressure. The residue was triturated with Et₂O (50 mL) then filtered. The filter cake was washed with Et₂O (50 mL) and MeCN (50 mL) then dried under vacuum to afford the title compound (1.50 g, 66%) as an off grey solid; ¹H NMR (400 MHz, DMSO, 30° C.) 1.43 (9H, s), 1.93-2 (1H, m), 2.38 (1H, dd), 2.61 (1H, s), 2.85-2.95 (1H, m), 3.27 (4H, s), 3.43-3.54 (4H, m), 4.34 (2H, d), 5.05 (1H, dd), 7.08 (2H, d), 7.54 (1H, d), 10.92 (1H, s); m/z: ES+ [M+H]⁺ 429.3.

### Intermediate 1k: 3-(1-Oxo-5-(piperazin-1-yl)isoindolin-2-yl)piperidine-2,6-dione, hydrochloride

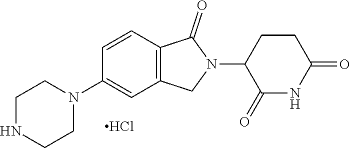 [SMILES: O=C1CCC(N2Cc3cc(N4CCNCC4)ccc3C2=O)C(=O)N1 ; Cl]

4M HCl in dioxane (8.75 mL, 35.0 mmol) was added to tert-butyl-4-(2-(2,6-dioxopiperidin-3-yl)-1-oxoisoindolin-5-yl)piperazine-1-carboxylate (1.50 g, 3.50 mmol) in 1,4-dioxane (2 mL) at RT and the reaction stirred for 1 h. EtOAc (5 mL) was added and the reaction mixture stirred for 10 mins. The resulting precipitate was collected by filtration and the solid washed with EtOAc (2×5 mL) and then dried under vacuum to afford the title compound (1.08 g, 85%) as a dark grey solid (HCl salt); ¹H NMR (400 MHz, DMSO, 30° C.) 1.97 (1H, dd), 2.36-2.44 (1H, m), 2.60 (1H, d), 2.84-2.99 (1H, m), 3.23 (4H, s), 3.5-3.57 (4H, m), 4.27 (1H, s), 4.34 (1H, s), 5.06 (1H, dd), 7.11-7.18 (2H, m), 7.59 (1H, d), 9.17 (2H, s), 10.93 (1H, s); m/z: ES+ [M+H]⁺ 329.0.

### Example 1: 3-[5-[4-[[1-[5-[(1R,3R)-2-(2-Fluoro-2-methyl-propyl)-3-methyl-1,3,4,9-tetrahydropyrido[3,4-b]indol-1-yl]pyrimidin-2-yl]-4-piperidyl]methyl]piperazin-1-yl]-1-oxo-isoindolin-2-yl]piperidine-2,6-dione

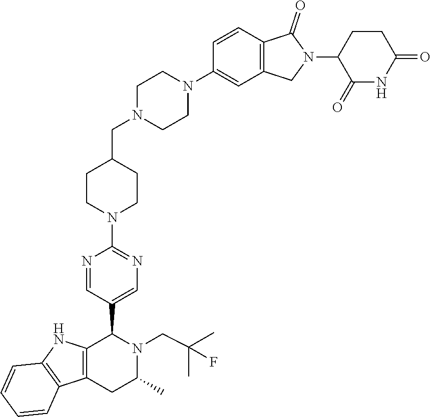 [SMILES: C[C@@H]1Cc2c([nH]c3ccccc23)[C@@H](c2cnc(N3CCC(CN4CCN(c5ccc6c(c5)CN(C5CCC(=O)NC5=O)C6=O)CC4)CC3)nc2)N1CC(C)(C)F]

1-(5-((1R,3R)-2-(2-Fluoro-2-methylpropyl)-3-methyl-2,3,4,9-tetrahydro-1H-pyrido[3,4-b]indol-1-yl)pyrimidin-2-yl)piperidine-4-carbaldehyde (58 mg, 0.13 mmol), sodium acetate (32 mg, 0.39 mmol) and 3-(1-oxo-5-(piperazin-1-yl)isoindolin-2-yl)piperidine-2,6-dione, HCl (47.4 mg, 0.13 mmol) were dissolved in DCM (5 mL) and MeOH (1 mL) and stirred for 10 mins. Sodium cyanotrihydroborate (24 mg, 0.39 mmol) was added and the reaction stirred for 30 mins at RT. The reaction mixture was diluted with water (20 mL) and EtOAc (50 mL). The organics were separated, washed with brine (20 mL), dried over anhydrous Na₂SO₄, filtered and concentrated. The crude product was purified by preparative HPLC (Waters XSelect CSH C18 ODB column, 5p silica, 30 mm diameter, 100 mm length), using decreasingly polar mixtures of water (containing 0.1% formic acid) and MeCN as eluents to the title compound (9 mg, 9%) as a pale yellow solid; ¹H NMR (400 MHz, CDCl₃, 30° C.) 1.10 (3H, d), 1.20 (2H, dd), 1.30 (3H, d), 1.47 (3H, d), 1.86 (4H, d), 2.19 (1H, dtd), 2.27 (2H, d), 2.29-2.38 (1H, m), 2.48-2.56 (1H, m), 2.58-2.63 (4H, m), 2.65 (2H, s), 2.76-2.94 (4H, m), 3.27 (1H, s), 3.29-3.38 (4H, m), 4.25 (1H, d), 4.41 (1H, d), 4.73 (2H, d), 5.00 (1H, s), 5.19 (1H, dd), 6.87 (1H, s), 6.99 (1H, dd), 7.14 (2H, dtd), 7.29 (1H, s), 7.48-7.54 (1H, m), 7.64-7.75 (2H, m), 7.95 (1H, d), 8.18 (2H, s); m/z: ES+ [M+H]⁺ 762.3.

### Intermediate 2a: Benzyl 4-(2-hydroxyethyl)piperidine-1-carboxylate

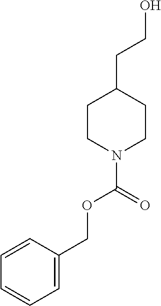 [SMILES: O=C(OCc1ccccc1)N1CCC(CCO)CC1]

To a solution of 2-(piperidin-4-yl)ethan-1-ol (5.00 g, 38.7 mmol) in DCM (100 mL) was added sodium carbonate (18.46 g, 174.1 mmol) in water (100 mL) at 0° C. and benzyl carbonochloridate (6.08 mL, 42.6 mmol) was added dropwise. The mixture was stirred for 6 h at RT and then diluted with water (100 mL) and extracted with DCM (2×100 mL). The combined organic layers were washed with brine (100 mL), dried over anhydrous Na₂SO₄, filtered and concentrated under reduced pressure. The crude product was purified by flash silica chromatography, elution gradient 0 to 70% EtOAc in heptane to afford the title compound (8.34 g, 82%) as a pale yellow oil; ¹H NMR (400 MHz, CDCl₃, 30° C.) 1.15 (2H, qd), 1.43 (1H, t), 1.52 (2H, q), 1.62 (1H, dddd), 1.69 (2H, t), 2.78 (2H, t), 3.69 (2H, q), 4.14 (2H, dd), 5.12 (2H, s), 7.28-7.45 (5H, m); m/z: ES+ [M+H]⁺ 264.3.

### Intermediate 2b: Benzyl 4-(2-oxoethyl)piperidine-1-carboxylate

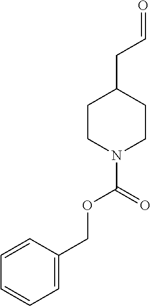 [SMILES: O=CCC1CCN(C(=O)OCc2ccccc2)CC1]

To a solution of benzyl 4-(2-hydroxyethyl)piperidine-1-carboxylate (8.34 g, 31.7 mmol) in DCM (150 mL) at 0° C. was added 3-oxo-115-benzo[d][1,2]iodaoxole-1,1,1(3H)-triyl triacetate (14.78 g, 34.84 mmol). The reaction was stirred at RT for 3 h and quenched by the addition of sat. NaHCO₃ solution (50 mL) and filtered to remove solid residue. The solid residue was washed with DCM (50 mL). The organic layer was separated and washed with brine (20 mL×2), dried over Na₂SO₄, filtered and concentrated. The crude product was purified by flash silica chromatography, elution gradient 0 to 50% EtOAc in heptane to afford the title compound (5.20 g, 63%) as a colourless oil; ¹H NMR (400 MHz, CDCl₃, 30° C.) 1.20 (2H, q), 1.71 (2H, d), 2.07 (1H, tq), 2.38 (2H, dd), 2.82 (2H, t), 4.16 (2H, s), 5.12 (2H, s), 7.31-7.43 (5H, m), 9.77 (1H, t); m/z: ES+ [M+H]⁺ 262.2.

### Intermediate 2c: Benzyl 4-(2,2-dimethoxyethyl)piperidine-1-carboxylate

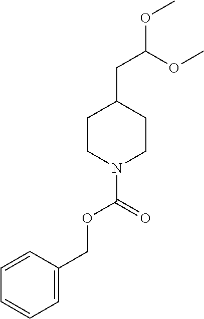 [SMILES: COC(CC1CCN(C(=O)OCc2ccccc2)CC1)OC]

To a solution of benzyl 4-(2-oxoethyl)piperidine-1-carboxylate (5.20 g, 19.9 mmol) in MeOH (60 mL) was added trimethoxymethane (10.9 mL, 99.5 mmol) and 4-methylbenzenesulfonic acid (0.17 g, 0.99 mmol) at 15° C. The mixture was stirred at this temperature for 1 h. The reaction was quenched by addition of water (50 mL) and diluted with DCM (100 mL). The organic layer was washed with brine (20 mL×3), dried over Na₂SO₄, filtered and concentrated under reduced pressure to afford the title compound (5.90 g, 96%) as a colourless oil; ¹H NMR (400 MHz, CDCl₃, 30° C.) 1.04-1.25 (2H, m), 1.5-1.57 (2H, m), 1.57-1.63 (1H, m), 1.68 (2H, t), 2.78 (2H, t), 3.31 (6H, s), 4.15 (2H, d), 4.46 (1H, t), 5.12 (2H, s), 7.27-7.37 (5H, m).

### Intermediate 2d: 4-(2,2-Dimethoxyethyl)piperidine

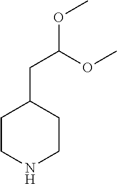

Dihydroxypalladium 10 wt % (0.540 g, 0.38 mmol) was added to benzyl 4-(2,2-dimethoxyethyl)piperidine-1-carboxylate (5.90 g, 19.2 mmol) in MeOH (60 mL) at 20° C. under N₂ in a steel pressured reactor. The resulting suspension was purged with N₂ and H₂ and stirred at 20° C. at 4 atm for 3 days. The reaction mixture was filtered over celite and the cake washed with MeOH (250 mL). The filtrate was concentrated to afford the title compound (3.14 g, 94%) as a colorless oil; ¹H NMR (400 MHz, CDCl₃, 30° C.) 1.15-1.28 (2H, m), 1.53-1.56 (2H, m), 1.72 (2H, d), 2.63 (2H, td), 3.04-3.14 (2H, m), 3.31 (8H, s), 4.47 (1H, t).

### Intermediate 2e: (1R,3R)-1-(2-(4-(2,2-Dimethoxyethyl)piperidin-1-yl)pyrimidin-5-yl)-2-(2-fluoro-2-methylpropyl)-3-methyl-2,3,4,9-tetrahydro-1H-pyrido[3,4-b]indole

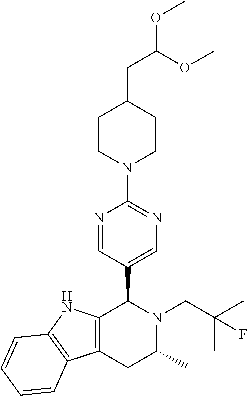 [SMILES: COC(CC1CCN(c2ncc([C@@H]3c4[nH]c5ccccc5c4C[C@@H](C)N3CC(C)(C)F)cn2)CC1)OC]

(1R,3R)-1-(2-Chloropyrimidin-5-yl)-2-(2-fluoro-2-methylpropyl)-3-methyl-2,3,4,9-tetrahydro-1H-pyrido[3,4-b]indole (2.5 g, 6.7 mmol), 4-(2,2-dimethoxyethyl)piperidine (1.2 g, 6.7 mmol) and DIPEA (3.5 mL, 20.1 mmol) were stirred in DMF (50 mL) at 90° C. for 4 h. The reaction mixture was cooled to RT and diluted with EtOAc (25 mL) and water (25 mL). The organics were separated and washed with brine (25 mL), dried over anhydrous Na₂SO₄, filtered and concentrated under reduced pressure. The crude product was purified by flash silica chromatography, elution gradient 0 to 100% EtOAc in heptane to afford the title compound (2.15 g, 63%) as a white solid; ¹H NMR (400 MHz, CDCl₃, 30° C.) 1.09 (3H, d), 1.19 (2H, td), 1.29 (3H, d), 1.46 (3H, d), 1.56 (2H, d), 1.63-1.73 (1H, m), 1.77 (2H, d), 2.48-2.62 (2H, m), 2.68 (2H, d), 2.86 (2H, td), 3.27 (1H, s), 3.32 (6H, s), 4.50 (1H, t), 4.70 (2H, d), 4.99 (1H, s), 7.08-7.19 (2H, m), 7.27 (1H, d), 7.51 (1H, d), 7.65 (1H, s), 8.17 (2H, s); m/z: ES+ [M+H]⁺ 510.2.

### Intermediate 2f: 2-(1-(5-((1R,3R)-2-(2-Fluoro-2-methylpropyl)-3-methyl-2,3,4,9-tetrahydro-1H-pyrido[3,4-b]indol-1-yl)pyrimidin-2-yl)piperidin-4-yl)acetaldehyde

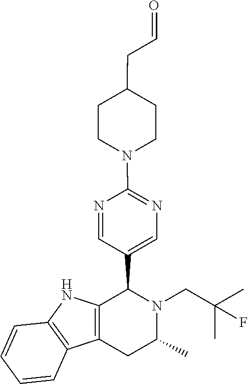 [SMILES: C[C@@H]1Cc2c([nH]c3ccccc23)[C@@H](c2cnc(N3CCC(CC=O)CC3)nc2)N1CC(C)(C)F]

(1R,3R)-1-(2-(4-(2,2-Dimethoxyethyl)piperidin-1-yl)pyrimidin-5-yl)-2-(2-fluoro-2-methylpropyl)-3-methyl-2,3,4,9-tetrahydro-1H-pyrido[3,4-b]indole (2.0 g, 3.9 mmol) was dissolved in 1,4-dioxane (30 mL) and formic acid (20 mL) and warmed to 45° C. for 1.5 h. The solvent was removed under reduced pressure. The crude product was purified by preparative HPLC (Waters XSelect CSH C18 ODB column, 5p silica, 30 mm diameter, 100 mm length), using decreasingly polar mixtures of water (containing 0.1% NH3) and MeCN as eluents to afford the title compound (1.06 g, 58%) as a pale yellow solid; ¹H NMR (400 MHz, CDCl₃, 30° C.) 1.10 (3H, d), 1.19-1.34 (5H, m), 1.47 (3H, d), 1.79 (2H, d), 2.17 (1H, ddt), 2.40 (2H, dd), 2.46-2.62 (2H, m), 2.68 (2H, d), 2.91 (2H, td), 3.26 (1H, s), 4.72 (2H, d), 5.00 (1H, s), 7.14 (2H, dtd), 7.28 (1H, d), 7.51 (1H, d), 7.66 (1H, s), 8.18 (2H, s), 9.80 (1H, t); m/z: ES+ [M+H]⁺ 464.0.

### Example 2: 3-[5-[4-[2-[1-[5-[(1R,3R)-2-(2-Fluoro-2-methyl-propyl)-3-methyl-1,3,4,9-tetrahydropyrido[3,4-b]indol-1-yl]pyrimidin-2-yl]-4-piperidyl]ethyl]piperazin-1-yl]-1-oxo-isoindolin-2-yl]piperidine-2,6-dione

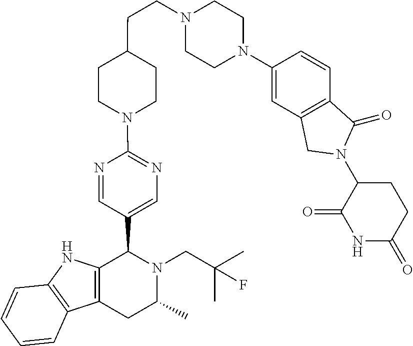 [SMILES: C[C@@H]1Cc2c([nH]c3ccccc23)[C@@H](c2cnc(N3CCC(CCN4CCN(c5ccc6c(c5)CN(C5CCC(=O)NC5=O)C6=O)CC4)CC3)nc2)N1CC(C)(C)F]

2-(1-(5-((1R,3R)-2-(2-Fluoro-2-methylpropyl)-3-methyl-2,3,4,9-tetrahydro-1H-pyrido[3,4-b]indol-1-yl)pyrimidin-2-yl)piperidin-4-yl)acetaldehyde (1.05 g, 2.26 mmol), sodium acetate (0.557 g, 6.79 mmol) and 3-(1-oxo-5-(piperazin-1-yl)isoindolin-2-yl)piperidine-2,6-dione, HCl (0.826 g, 2.26 mmol) were dissolved in DCM (25 mL) and MeOH (5 mL) and stirred for 1 h. Sodium cyanotrihydroborate (0.427 g, 6.79 mmol) was added and the reaction stirred for 30 mins at RT. The reaction mixture was diluted with water (20 mL) and EtOAc (50 mL). The organics were separated, washed with brine (20 mL), dried over anhydrous Na₂SO₄, filtered and concentrated. The crude product was purified by preparative HPLC (Waters XSelect CSH C18 ODB column, 5p silica, 30 mm diameter, 100 mm length), using decreasingly polar mixtures of water (containing 0.1% NH3) and MeCN as eluents. The HPLC fractions were extracted with DCM (500 mL). The combined organics were washed with brine, passed through a phase separating cartridge and concentrated. The product was further purified by flash silica chromatography, elution gradient 0 to 100% EtOAc in heptane, then 20% EtOH in EtOAc to afford the title compound (0.326 g, 19%) as a white solid; ¹H NMR (400 MHz, CDCl₃, 30° C.) 0.94 (0H, t), 1.10 (3H, d), 1.15-1.26 (2H, m), 1.30 (3H, d), 1.41-1.53 (5H, m), 1.58 (1H, s), 1.77 (2H, d), 2.04 (0H, s), 2.19 (1H, dtd), 2.32 (1H, qd), 2.41-2.49 (2H, m), 2.48-2.63 (6H, m), 2.68 (2H, d), 2.84 (4H, tdd), 3.19-3.39 (5H, m), 4.12 (0H, q), 4.25 (1H, d), 4.41 (1H, d), 4.71 (2H, d), 4.99 (1H, s), 5.18 (1H, dd), 5.30 (0H, s), 6.87 (1H, s), 6.99 (1H, dd), 7.13 (2H, dtd), 7.27 (1H, d), 7.51 (1H, d), 7.72 (2H, t), 7.92 (1H, s), 8.17 (2H, s); m/z: ES+ [M+H]⁺ 776.5.

### Intermediate 3a: tert-Butyl 4-(2-(2,6-dioxopiperidin-3-yl)-1,3-dioxoisoindolin-5-yl)piperazine-1-carboxylate

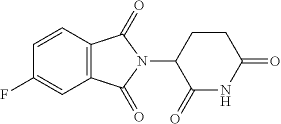 [SMILES: O=C1CCC(N2C(=O)c3ccc(F)cc3C2=O)C(=O)N1]

To a solution of 5-fluoroisobenzofuran-1,3-dione (7.50 g, 45.2 mmol) in acetic acid (100 mL) was added sodium acetate (7.41 g, 90.3 mmol) and 3-aminopiperidine-2,6-dione hydrochloride (7.43 g, 45.2 mmol). The mixture was stirred at 120° C. for 18 h. The reaction mixture was concentrated under reduced pressure. The residue was poured into water (200 mL) and stirred for 10 mins. The mixture was filtered, washed with water (2×50 mL) and dried under vacuum to afford the title compound (11.8 g, 94%) as a white solid; ¹H NMR (400 MHz, DMSO, 30° C.) 2.03-2.12 (1H, m), 2.52-2.66 (2H, m), 2.90 (1H, ddd), 5.17 (1H, dd), 7.73 (1H, ddd), 7.85 (1H, dd), 8.01 (1H, dd), 11.12 (1H, s); m/z: ES− [M−H]⁻ 275.1.

### Intermediate 3b: tert-Butyl 4-(2-(2,6-dioxopiperidin-3-yl)-1,3-dioxoisoindolin-5-yl)piperazine-1-carboxylate

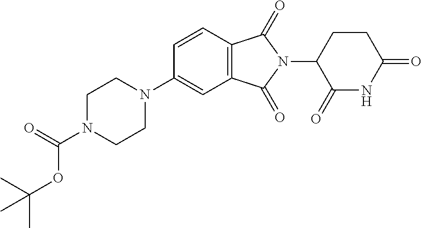 [SMILES: CC(C)(C)OC(=O)N1CCN(c2ccc3c(c2)C(=O)N(C2CCC(=O)NC2=O)C3=O)CC1]

tert-Butyl piperazine-1-carboxylate (2.97 g, 15.9 mmol), 2-(2,6-dioxopiperidin-3-yl)-5-fluoroisoindoline-1,3-dione (4.00 g, 14.5 mmol), DIPEA (7.80 mL, 43.4 mmol) and NMP (60 mL) were heated in a microwave reactor at 140° C. for 2 h. The reaction mixture was cooled to RT, diluted with water (100 mL) and extracted with EtOAc (2×100 mL). The combined organics were washed with brine (2×50 mL), dried over anhydrous Na₂SO₄, filtered and concentrated. The crude product was purified by flash silica chromatography, elution gradient 0 to 100% EtOAc in heptane to afford the title compound (3.95 g, 62%) as a yellow solid; ¹H NMR (400 MHz, DMSO, 30° C.) 1.43 (9H, s), 2.03 (1H, ddd), 2.53-2.65 (2H, m), 2.77-2.97 (1H, m), 3.48 (8H, s), 5.08 (1H, dd), 7.25 (1H, dd), 7.35 (1H, d), 7.70 (1H, d), 11.06 (1H, s).

### Intermediate 3c: 2-(2,6-Dioxopiperidin-3-yl)-5-(piperazin-1-yl)isoindoline-1,3-dione, hydrochloride

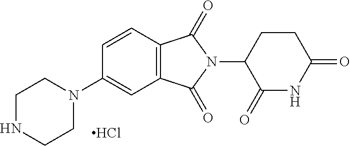 [SMILES: O=C1CCC(N2C(=O)c3ccc(N4CCNCC4)cc3C2=O)C(=O)N1 ; Cl]

4M HCl in dioxane (22.3 mL, 89.3 mmol) was added to tert-butyl 4-(2-(2,6-dioxopiperidin-3-yl)-1,3-dioxoisoindolin-5-yl)piperazine-1-carboxylate (3.95 g, 8.93 mmol) in DCM (100 mL) at RT. The reaction was stirred at RT for 18 h. The solvents were removed under reduced pressure to afford the title compound (3.40 g, 100%) as a pale yellow solid (HCl salt); ¹H NMR (400 MHz, DMSO, 30° C.) 2.04 (1H, ddd), 2.55-2.65 (2H, m), 2.90 (1H, ddd), 3.22 (4H, s), 3.67-3.73 (4H, m), 5.09 (1H, dd), 7.33 (1H, dd), 7.46 (1H, d), 7.75 (1H, d), 9.22 (2H, s), 11.07 (1H, s); m/z: ES+ [M+H]⁺ 343.2.

### Example 3: 2-[2,6-Dioxo3-piperidyl]-5-[4-[[1-[5-[(1R,3R)-2-(2-fluoro-2-methyl-propyl)-3-methyl-1,3,4,9-tetrahydropyrido[3,4-b]indol-1-yl]pyrimidin-2-yl]-4-piperidyl]methyl]piperazin-1-yl]isoindoline-1,3-dione. Formic Acid Salt

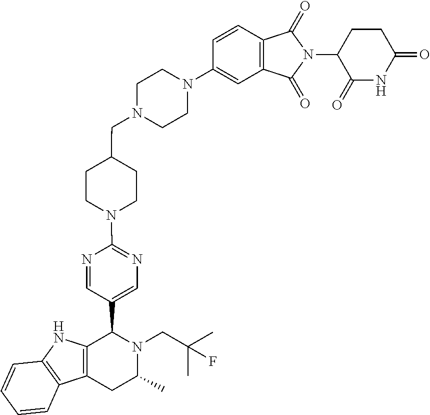 [SMILES: C[C@@H]1Cc2c([nH]c3ccccc23)[C@@H](c2cnc(N3CCC(CN4CCN(c5ccc6c(c5)C(=O)N(C5CCC(=O)NC5=O)C6=O)CC4)CC3)nc2)N1CC(C)(C)F]

1-(5-((1R,3R)-2-(2-Fluoro-2-methylpropyl)-3-methyl-2,3,4,9-tetrahydro-1H-pyrido[3,4-b]indol-1-yl)pyrimidin-2-yl)piperidine-4-carbaldehyde (0.022 g, 0.050 mmol), sodium acetate (0.012 g, 0.15 mmol) and 2-(2,6-dioxopiperidin-3-yl)-5-(piperazin-1-yl)isoindoline-1,3-dione, HCl (0.019 g, 0.050 mmol) were dissolved in DCM (2 mL) and MeOH (0.5 mL) and stirred for 10 mins. Sodium cyanotrihydroborate (9.2 mg, 0.15 mmol) was added and the reaction stirred for 30 mins at RT. The reaction mixture was diluted with water (20 mL) and EtOAc (50 mL). The organics were separated, washed with brine (20 mL), dried over anhydrous Na₂SO₄, filtered and concentrated. The product was purified by preparative HPLC (Waters XSelect CSH C18 ODB column, 5p silica, 30 mm diameter, 100 mm length), using decreasingly polar mixtures of water (containing 0.1% formic acid) and MeCN as eluents to afford the title compound (0.012 g, 30%) as a pale yellow solid (formate salt); ¹H NMR (400 MHz, CDCl₃, 30° C.) 1.10 (3H, d), 1.16-1.23 (2H, m), 1.30 (3H, d), 1.47 (3H, d), 1.86 (4H, d), 2.08-2.18 (1H, m), 2.27 (2H, d), 2.49-2.56 (1H, m), 2.56-2.6 (4H, m), 2.67 (2H, d), 2.7-2.77 (1H, m), 2.79 (1H, dd), 2.88 (3H, t), 3.26 (1H, s), 3.4-3.45 (4H, m), 4.74 (2H, d), 4.93 (1H, dd), 5.00 (1H, s), 7.05 (1H, dd), 7.11 (1H, td), 7.14-7.19 (1H, m), 7.27 (2H, dd), 7.5-7.53 (1H, m), 7.63 (1H, s), 7.69 (1H, d), 7.93 (1H, s), 8.02 (1H, s), 8.19 (2H, s); m/z: ES+ [M+H]⁺ 776.2.

### Intermediate 4a: 2-((1-(5-((1R,3R)-2-(2-Fluoro-2-methylpropyl)-3-methyl-2,3,4,9-tetrahydro-1H-pyrido[3,4-b]indol-1-yl)pyrimidin-2-yl)piperidin-4-yl)oxy)ethan-1-ol

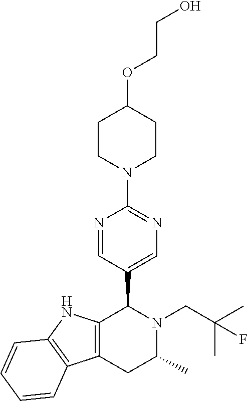 [SMILES: C[C@@H]1Cc2c([nH]c3ccccc23)[C@@H](c2cnc(N3CCC(OCCO)CC3)nc2)N1CC(C)(C)F]

2-(Piperidin-4-yloxy)ethan-1-ol (193 mg, 1.33 mmol), DIPEA (0.580 mL, 3.33 mmol) and (1R,3R)-1-(2-chloropyrimidin-5-yl)-2-(2-fluoro-2-methylpropyl)-3-methyl-2,3,4,9-tetrahydro-1H-pyrido[3,4-b]indole (414 mg, 1.11 mmol) were dissolved in DMF (3.1 mL) and sealed into a microwave tube. The reaction was heated to 120° C. for 15 minutes in the microwave reactor. The temperature was increased to 140° C. and the reaction mixture was stirred for a further 7 minutes. The reaction mixture was diluted with MeOH (1 mL) and was purified by preparative HPLC (Waters XSelect CSH C18 ODB column, 5p silica, 30 mm diameter, 100 mm length), using decreasingly polar mixtures of water (containing 1% by volume NH30H (28-30% in H₂O)) and MeCN as eluents to afford the title compound (500 mg, 94%) as a yellow dry film. ¹H NMR (400 MHz, CDCl₃, 30° C.) 1.10 (3H, d), 1.29 (3H, d), 1.47 (3H, d), 1.54-1.66 (2H, m), 1.94 (2H, dq), 1.98-2.03 (1H, m), 2.46-2.74 (4H, m), 3.19--3.31 (1H, m), 3.37 (2H, ddd), 3.54-3.64 (3H, m), 3.69-3.79 (2H, m), 4.29 (2H, dt), 4.99 (1H, s), 7.13 (2H, dtd), 7.26 (1H, d), 7.51 (1H, d), 7.74 (1H, s), 8.18 (2H, s); m/z: ES− [M−H]⁻ 480.3.

### Example 4: 3-[5-[4-[2-[[1-[5-[(1R,3R)-2-(2-Fluoro-2-methyl-propyl)-3-methyl-1,3,4,9-tetrahydropyrido[3,4-b]indol-1-yl]pyrimidin-2-yl]-4-piperidyl]oxy]ethyl]piperazin-1-yl]-1-oxo-isoindolin-2-yl]piperidine-2,6-dione

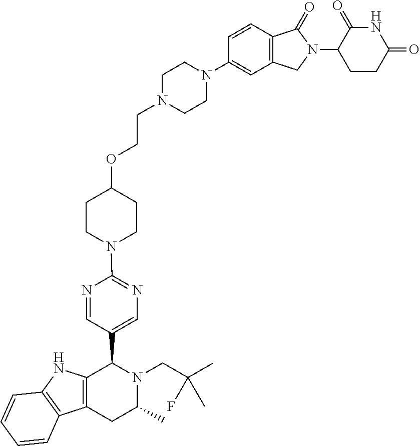 [SMILES: C[C@@H]1Cc2c([nH]c3ccccc23)[C@@H](c2cnc(N3CCC(OCCN4CCN(c5ccc6c(c5)CN(C5CCC(=O)NC5=O)C6=O)CC4)CC3)nc2)N1CC(C)(C)F]

SO₃-pyridine complex (136 mg, 0.86 mmol) was added to a solution of 2-((1-(5-((1R,3R)-2-(2-fluoro-2-methylpropyl)-3-methyl-2,3,4,9-tetrahydro-1H-pyrido[3,4-b]indol-1-yl)pyrimidin-2-yl)piperidin-4-yl)oxy)ethan-1-ol (206 mg, 0.43 mmol) and triethylamine (0.119 mL, 0.86 mmol) in DCM (1.0 mL)-DMSO (1.0 mL) at 0° C. The reaction was allowed to warm to RT for 18 hours. The reaction was diluted with DCM (20 mL) and water (20 mL) and the layers were separated. The organic layer was washed with brine (20 mL), dried and evaporated to afford crude aldehyde product that dissolved in in DCM (2.8 mL) and MeOH (1.4 mL). 3-(1-Oxo-5-(piperazin-1-yl)isoindolin-2-yl)piperidine-2,6-dione, 2HCl (184 mg, 0.46 mmol), sodium acetate (103 mg, 1.25 mmol) were then added and the resulting mixture was stirred at room temperature under N₂ for 45 minutes. Sodium cyanotrihydroborate (79 mg, 1.25 mmol) was added and the resulting mixture was stirred at 20° C. for 1 h. The reaction mixture was diluted with methanol (2 mL), filtered and purified by preparative HPLC (Waters CSH C18 OBD column, 30×100 mm id, 5 micron particle size), using decreasingly polar mixtures of water (containing 0.1% NH₃aq) and MeCN as eluents. Fractions containing the desired compound were extracted with DCM (4×30 mL). The combined organic phase were dried over a phase separator and concentrated to afford the title compound (91 mg, 28%) as a grey solid; ¹H NMR (400 MHz, CDCl₃, 30° C.) 1.10 (3H, d), 1.30 (3H, d), 1.47 (3H, d), 1.56-1.66 (2H, m), 1.85-1.98 (2H, m), 2.11-2.22 (1H, m), 2.22-2.42 (1H, m), 2.47-2.63 (2H, m), 2.68 (7H, dq), 2.74-2.97 (2H, m), 3.2-3.29 (1H, m), 3.3-3.35 (4H, m), 3.39 (2H, ddd), 3.47-3.51 (1H, m), 3.52-3.61 (1H, m), 3.68 (2H, t), 4.19-4.31 (3H, m), 4.40 (1H, d), 5.00 (1H, s), 5.18 (1H, ddd), 6.86 (1H, s), 6.98 (1H, dd), 7.08-7.2 (2H, m), 7.28 (1H, d), 7.48-7.55 (1H, m), 7.66-7.75 (2H, m), 7.89 (1H, s), 8.18 (2H, s); m/z: ES+ [M+H]⁺ 792.7.

### Intermediate 5a: 5-(3,5-Difluoro-4-((1R,3R)-2-(2-fluoro-2-methylpropyl)-3-methyl-2,3,4,9-tetrahydro-1H-pyrido[3,4-b]indol-1-yl)phenoxy)pentan-1-ol

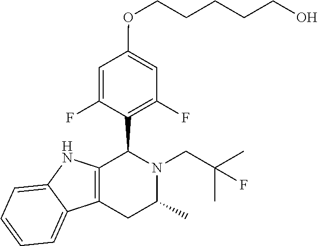 [SMILES: C[C@@H]1Cc2c([nH]c3ccccc23)[C@@H](c2c(F)cc(OCCCCCO)cc2F)N1CC(C)(C)F]

Rock Phos Pd G3 (0.086 g, 0.10 mmol) was added in one portion to a degassed mixture of pentane-1,5-diol (1.29 mL, 12.3 mmol), (1R,3R)-1-(4-bromo-2,6-difluorophenyl)-2-(2-fluoro-2-methylpropyl)-3-methyl-2,3,4,9-tetrahydro-1H-pyrido[3,4-b]indole (1.00 g, 2.05 mmol) and cesium carbonate (2.34 g, 7.18 mmol) in toluene (10 mL) at 20° C. under N₂. The resulting mixture was stirred at 80° C. for 18 h. The reaction was allowed to cool to RT and diluted with EtOAc (50 mL) and water (15 mL). The organic layer was collected and washed with sat. brine solution (20 mL), dried over MgSO₄, filtered and evaporated to afford crude product as an orange gum. The crude product was purified by flash silica chromatography, elution gradient 0 to 80% EtOAc in heptane to afford the title compound (0.53 g, 55%) as a white solid; ¹H NMR (400 MHz, CDCl₃, 30° C.) 1.10 (3H, d), 1.14-1.33 (7H, m), 1.49-1.59 (2H, m), 1.59-1.69 (2H, m), 1.81 (2H, dt), 2.39 (1H, dd), 2.60 (1H, dd), 2.86 (1H, dd), 3.09 (1H, dd), 3.68 (3H, q), 3.92 (2H, t), 5.18 (1H, s), 6.35-6.43 (2H, m), 7.05-7.14 (2H, m), 7.19-7.24 (1H, m), 7.40 (1H, s), 7.47-7.55 (1H, m); m/z: ES− [M−H]⁻ 473.3.

### Intermediate 5b: 5-(3,5-Difluoro-4-((1R,3R)-2-(2-Fluoro-2-methylpropyl)-3-methyl-2,3,4,9-tetrahydro-1H-pyrido[3,4-b]indol-1-yl)phenoxy)pentanal

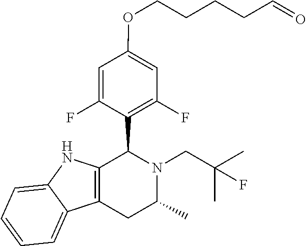 [SMILES: C[C@@H]1Cc2c([nH]c3ccccc23)[C@@H](c2c(F)cc(OCCCCC=O)cc2F)N1CC(C)(C)F]

SO₃-pyridine complex (344 mg, 2.16 mmol) was added to a solution of 5-(3,5-difluoro-4-((1R,3R)-2-(2-fluoro-2-methylpropyl)-3-methyl-2,3,4,9-tetrahydro-1H-pyrido[3,4-b]indol-1-yl)phenoxy)pentan-1-ol (455 mg, 0.96 mmol) and triethylamine (0.334 mL, 2.40 mmol) in DCM (1.6 mL)-DMSO (1.6 mL) at 0° C. The reaction was allowed to warm to RT for 18 h. A second addition of SO₃-pyridine complex (344 mg, 2.16 mmol) was added to the reaction. The reaction was diluted with DCM (20 mL) and water (20 mL), then the layers were separated. The organic layer was washed with brine, then dried and evaporated to afford crude product. The crude product was purified by flash silica chromatography, elution gradient 0 to 60% EtOAc in heptane to afford the title compound (186 mg, 41%) as a colourless oil; m/z: ES+ [M+H]+ 473.2.

### Example 5: 3-[5-[4-[5-[3,5-Difluoro-4-[(1R,3R)-2-(2-fluoro-2-methyl-propyl)-3-methyl-1,3,4,9-tetrahydropyrido[3,4-b]indol-1-yl]phenoxy]pentyl]piperazin-1-yl]-1-oxo-isoindolin-2-yl]piperidine-2,6-dione

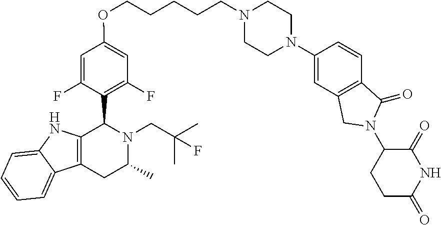 [SMILES: C[C@@H]1Cc2c([nH]c3ccccc23)[C@@H](c2c(F)cc(OCCCCCN3CCN(c4ccc5c(c4)CN(C4CCC(=O)NC4=O)C5=O)CC3)cc2F)N1CC(C)(C)F]

A solution of 3-(1-oxo-5-(piperazin-1-yl)isoindolin-2-yl)piperidine-2,6-dione, HCl (81 mg, 0.19 mmol), 5-(3,5-difluoro-4-((1R,3R)-2-(2-fluoro-2-methylpropyl)-3-methyl-2,3,4,9-tetrahydro-1H-pyrido[3,4-b]indol-1-yl)phenoxy)pentanal (100 mg, 0.15 mmol) and sodium acetate (37 mg, 0.44 mmol) in DCM (2 mL) and MeOH (1 mL) was stirred at RT under N₂ for 20 mins. Sodium cyanotrihydroborate (26 mg, 0.42 mmol) was added and the resulting solution was stirred at RT for 2 days. The reaction mixture was diluted with MeOH (3 mL), filtered and purified by preparative HPLC (Waters CSH C18 OBD column, 30×100 mm id, 5 micron particle size), using decreasingly polar mixtures of water (containing 0.1% NH₃aq) and MeCN as eluents. Fractions containing the desired compound were extracted with DCM (2×30 mL). The combined organic phase were dried over a phase separator and concentrated afford the title compound (108 mg, 93%) as a yellow dry film; ¹H NMR (400 MHz, CDCl₃, 30° C.) 1.10 (3H, d), 1.20 (6H, dd), 1.54 (2H, s), 1.60 (2H, s), 1.75-1.86 (2H, m), 2.15-2.24 (1H, m), 2.25-2.46 (4H, m), 2.56-2.64 (5H, m), 2.76-2.95 (3H, m), 3.09 (1H, d), 3.29-3.37 (4H, m), 3.68 (1H, s), 3.92 (2H, t), 4.25 (1H, d), 4.41 (1H, d), 5.14-5.23 (2H, m), 6.39 (2H, d), 6.87 (1H, s), 6.95-7.02 (1H, m), 7.05-7.14 (2H, m), 7.19-7.23 (1H, m), 7.39 (1H, s), 7.48-7.54 (1H, m), 7.73 (1H, d), 7.86 (1H, s); m/z: ES+ [M+H]⁺ 785.4.

### Intermediate 6a: tert-Butyl 4-((1-((benzyloxy)carbonyl)piperidin-4-yl)methyl)piperazine-1-carboxylate

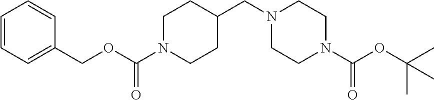 [SMILES: CC(C)(C)OC(=O)N1CCN(CC2CCN(C(=O)OCc3ccccc3)CC2)CC1]

Sodium triacetoxyborohydride (6.8 g, 32 mmol) was added in one portion to 1-Boc-piperazine (4.0 g, 21 mmol), 4-formyl-N-Cbz-piperidine (6.4 g, 26 mmol) and acetic acid (1.5 ml, 26 mmol) in dichloromethane (50 mL) at 20° C. under air. The resulting suspension was stirred at 20° C. for 2 hours. The reaction mixture was diluted with saturated aq. NaHCO₃ (60 mL), the layers were separated, and the aqueous layer was extracted with dichloromethane (3×40 mL). The combined organic layers were dried with MgSO₄, filtered and evaporated. The crude product was purified by flash silica chromatography, eluting with 50 to 70% EtOAc in heptane to afford the title compound (8.83 g, 98%) as a colourless oil; ¹H NMR (400 MHz, DMSO, 30° C.) 0.91-1.05 (2H, m), 1.40 (9H, s), 1.6-1.77 (3H, m), 2.12 (2H, d), 2.22-2.32 (4H, m), 2.79 (2H, s), 3.26-3.33 (4H, m), 3.99 (2H, d), 5.07 (2H, s), 7.28-7.44 (5H, m); m/z ES+ [M+H]+ 418.3.

### Intermediate 6b: tert-Butyl 4-(piperidin-4-ylmethyl)piperazine-1-carboxylate

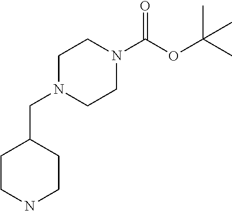

tert-Butyl 4-((1-((benzyloxy)carbonyl)piperidin-4-yl)methyl)piperazine-1-carboxylate (9.5 g, 23 mmol) and 10% palladium hydroxide on activated charcoal (3.20 g, 2.28 mmol) in ethanol (40 mL) were stirred under an atmosphere of hydrogen at 1 atm and 20° C. for 18 hours. The reaction mixture was filtered through celite and the solids washed through with EtOH. The filtrate was evaporated to dryness, dissolved in EtOH (40 mL) and 10% palladium hydroxide on activated charcoal (3.20 g, 2.28 mmol) added. The suspension was stirred under an atmosphere of hydrogen at 1 atm and 20° C. for 3 days. The reaction mixture was filtered through celite and the solids washed with EtOH (100 mL). The filtrate was evaporated to dryness to afford the title compound (5.61 g, 87%) as a grey solid; ¹H NMR (400 MHz, DMSO, 30° C.) 1.03-1.2 (2H, m), 1.40 (9H, s), 1.61-1.79 (3H, m), 2.11 (2H, d), 2.2-2.31 (4H, m), 2.63 (2H, td), 3.07 (2H, d), 3.22-3.36 (4H, m), exchangeable proton not observed; m/z: ES+ [M+H]+ 284.2.

### Intermediate 6c: tert-Butyl 4-((1-(5-((1R,3R)-2-(2-fluoro-2-methylpropyl)-3-methyl-2,3,4,9-tetrahydro-1H-pyrido[3,4-b]indol-1-yl)pyrimidin-2-yl)piperidin-4-yl)methyl)piperazine-1-carboxylate

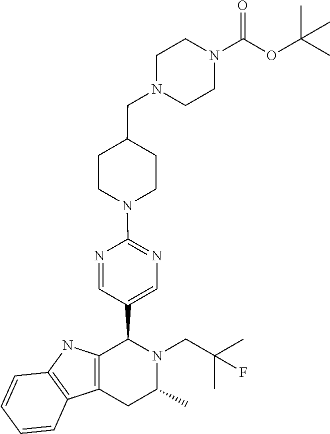 [SMILES: C[C@@H]1Cc2c([nH]c3ccccc23)[C@@H](c2cnc(N3CCC(CN4CCN(C(=O)OC(C)(C)C)CC4)CC3)nc2)N1CC(C)(C)F]

DIPEA (2.80 ml, 16.1 mmol) was added to (1R,3R)-1-(2-chloropyrimidin-5-yl)-2-(2-fluoro-2-methylpropyl)-3-methyl-2,3,4,9-tetrahydro-1H-pyrido[3,4-b]indole (3.0 g, 8.1 mmol) and tert-butyl 4-(piperidin-4-ylmethyl)piperazine-1-carboxylate (2.75 g, 9.70 mmol) in DMSO (25 mL) at 20° C. under air. The resulting suspension was stirred at 50° C. for 20 hours. The reaction was incomplete and further DIPEA (2.80 mL, 16.1 mmol) was added and the suspension was stirred at 50° C. for a further 8 hours. The reaction mixture was diluted with EtOAc (200 mL), and washed sequentially with water (4×50 mL) and saturated brine (20 mL). The organic layer was dried with MgSO₄, filtered and evaporated to afford crude product. The crude product was purified by flash silica chromatography, elution gradient 30 to 70% EtOAc in heptane to afford the title compound (3.52 g, 71%) as a white solid; ¹H NMR (400 MHz, DMSO, 30° C.) 1.00 (2H, d), 1.08 (3H, d), 1.28 (3H, d), 1.40 (12H, s), 1.75 (3H, d), 2.13 (2H, d), 2.26-2.32 (4H, m), 2.44-2.49 (1H, m), 2.6-2.9 (4H, m), 3.15 (1H, s), 3.30 (5H, s), 4.61 (2H, d), 4.91 (1H, s), 6.98 (1H, td), 7.06 (1H, td), 7.27 (1H, d), 7.43 (1H, d), 8.10 (2H, s), 10.71 (1H, s); m/z: ES+ [M+H]+ 620.5.

### Intermediate 6d: Benzyl 4-(dibutoxymethyl)piperidine-1-carboxylate

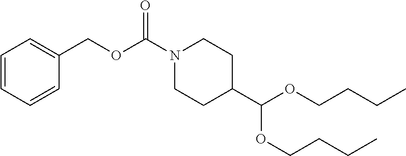 [SMILES: CCCCOC(OCCCC)C1CCN(C(=O)OCc2ccccc2)CC1]

4-Methylbenzenesulfonic acid, hydrate (0.1 g, 0.53 mmol) was added to benzyl 4-formylpiperidine-1-carboxylate (20 g, 81 mmol) in n-butanol (40 mL) at 20° C. under air. The resulting solution was stirred at 50° C. for 1 hour. The reaction was incomplete and magnesium sulfate (10.6 g, 88.1 mmol) was added and the suspension was stirred at 50° C. for a further 1 hour. The reaction was incomplete so the temperature was increased to 70° C. and the reaction mixture was stirred for a further 1 day. The reaction mixture was filtered and the filtrate collected into a vessel containing 2M aq. potassium carbonate (40 mL). The solids were washed with EtOAc (200 mL). The aqueous layer was removed and the organic layer washed sequentially with 2 M aq. potassium carbonate (2×40 mL) and saturated brine (2×20 mL). The organic layer was dried with MgSO₄, filtered and evaporated to afford crude product. The crude product was purified by flash silica chromatography, elution gradient 0 to 30% EtOAc in heptane to afford the title compound (20.5 g, 67%) as a colourless liquid; ¹H NMR (400 MHz, DMSO, 30° C.) 0.88 (6H, t), 1.11 (2H, qd), 1.27-1.4 (4H, m), 1.47 (4H, dq), 1.65 (2H, d), 1.73 (1H, dtt), 2.75 (2H, s), 3.37 (2H, dt), 3.54 (2H, dt), 3.95-4.06 (2H, m), 4.16 (1H, d), 5.07 (2H, s), 7.25-7.44 (5H, m); m/z: ES− M− 377.1.

### Intermediate 6e: 4-(Dibutoxymethyl)piperidine

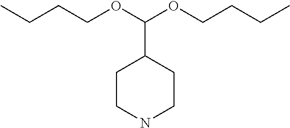 [SMILES: CCCCOC(OCCCC)C1CCNCC1]

Benzyl 4-(dibutoxymethyl)piperidine-1-carboxylate (20.5 g, 54.30 mmol) and 10% palladium hydroxide on activated charcoal (3.8 g, 2.71 mmol) in ethanol (120 mL) were stirred under an atmosphere of hydrogen at 1 atm and 20° C. for 3 days. The reaction mixture was filtered through celite rinsing the solids with EtOH (200 mL). The filtrate was evaporated to dryness to afford the title compound (13.1 g, 99%) as a colourless oil; ¹H NMR (400 MHz, DMSO, 30° C.) 0.88 (6H, t), 1.07 (2H, qd), 1.26-1.4 (4H, m), 1.41-1.51 (4H, m), 1.51-1.65 (3H, m), 2.05 (1H, s), 2.31-2.46 (2H, m), 2.90 (2H, d), 3.36 (2H, dt), 3.52 (2H, dt), 4.10 (1H, d).

### Intermediate 6f: 3-(5-Bromo-1-oxoisoindolin-2-yl)piperidine-2,6-dione

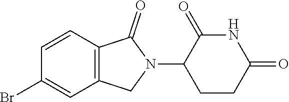 [SMILES: O=C1CCC(N2Cc3cc(Br)ccc3C2=O)C(=O)N1]

DIPEA (8.0 mL, 45.00 mmol) was added in one portion to a stirred solution of methyl 4-bromo-2-(bromomethyl)benzoate (4.62 g, 15 mmol) and 3-aminopiperidine-2,6-dione hydrochloride (3.70 g, 22.50 mmol) in acetonitrile (67 mL) at 20° C. under air. The resulting solution was stirred at 80° C. for 16 hours. The reaction mixture was cooled to 20° C. and filtered. The solid was washed with MeCN (20 mL) and diethyl ether (2×20 mL) to afford the title compound (3.80 g, 78%) as a blue solid; ¹H NMR (400 MHz, DMSO, 30° C.) 1.95-2.08 (1H, m), 2.34-2.46 (1H, m), 2.57-2.65 (1H, m), 2.91 (1H, ddd), 4.35 (1H, d), 4.48 (1H, d), 5.11 (1H, dd), 7.67 (1H, d), 7.72 (1H, dd), 7.83-7.96 (1H, m), 10.98 (1H, s); m/z: ES+ [M+H]+ 324.9.

### Intermediate 62: 3-[5-[4-(dibutoxymethyl)-1-piperidyl]-1-oxo-isoindolin-2-yl]piperidine-2,6-dione

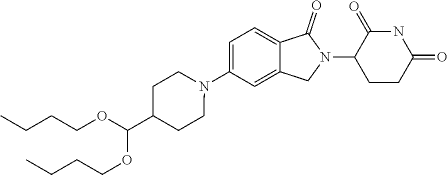 [SMILES: CCCCOC(OCCCC)C1CCN(c2ccc3c(c2)CN(C2CCC(=O)NC2=O)C3=O)CC1]

Pd-PEPPSI-IHept^{Cl} (1.13 g, 1.16 mmol) was added to a degassed mixture of 3-(5-bromo-1-oxo-isoindolin-2-yl)piperidine-2,6-dione (7.5 g, 23 mmol), 4-(dibutoxymethyl)piperidine (7.5 g, 31 mmol) and cesium carbonate (22.7 g, 69.6 mmol) in 1,4-dioxane (230 mL) at 40° C. under nitrogen. The resulting mixture was vacuum degassed, backfilling with nitrogen and stirred at 100° C. for 3 hours. The reaction mixture was cooled to room temperature, diluted with DCM (375 mL) and 10% aq. AcOH (250 mL), the layers were separated, and the aqueous layer was extracted with DCM (375 mL). The combined organic layers were washed sequentially with saturated NaHCO₃ (250 mL) and water (100 mL). Brine was added (100 mL). The mixture was filtered through celite and evaporated to dryness. The residue was diluted with DCM (150 mL), water (100 mL) and saturated brine (50 mL), the layers were separated, and the aqueous layer was extracted with DCM (2×125 mL). The combined organic layers were dried with MgSO₄, filtered and evaporated to afford crude product. The crude solid was triturated with EtOAc (75 mL) to give a solid which was collected by filtration, washed sequentially with EtOAc (2×25 mL), EtOAc:Et₂O (1:1; 20 mL), and Et₂O (20 mL) and dried under vacuum to afford the title compound (7.21 g, 64%) as a pale grey solid; ¹H NMR (400 MHz, DMSO, 30° C.) 0.89 (6H, t), 1.25-1.42 (6H, m), 1.43-1.55 (4H, m), 1.67-1.88 (3H, m), 1.97 (1H, ddt), 2.28-2.44 (1H, m), 2.55-2.65 (1H, m), 2.71-2.84 (2H, m), 2.90 (1H, ddd), 3.40 (2H, dt), 3.56 (2H, dt), 3.89 (2H, d), 4.18 (1H, s), 4.21 (1H, d), 4.32 (1H, d), 5.04 (1H, dd), 6.98-7.09 (2H, m), 7.50 (1H, d), 10.91 (1H, s); ES+ [M+H]+ 486.3.

### Example 6: 3-{5-[4-({4-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]piperazin-1-yl}methyl)piperazin-1-yl]-1-oxo-1,3-dihydro-2H-isoindol-2-yl}piperidine-2,6-dione

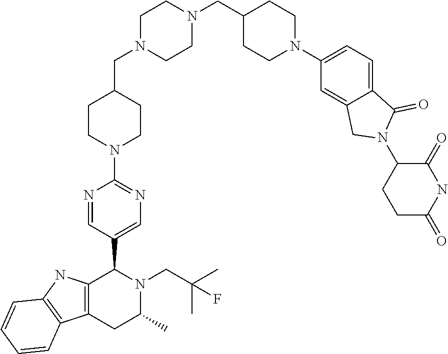 [SMILES: C[C@@H]1Cc2c([nH]c3ccccc23)[C@@H](c2cnc(N3CCC(CN4CCN(CC5CCN(c6ccc7c(c6)CN(C6CCC(=O)NC6=O)C7=O)CC5)CC4)CC3)nc2)N1CC(C)(C)F]

Formic acid (40 mL) was added to tert-butyl 4-((1-(5-((1R,3R)-2-(2-fluoro-2-methylpropyl)-3-methyl-2,3,4,9-tetrahydro-1H-pyrido[3,4-b]indol-1-yl)pyrimidin-2-yl)piperidin-4-yl)methyl)piperazine-1-carboxylate (3.43 g, 5.53 mmol) and 3-[5-[4-(dibutoxymethyl)-1-piperidyl]-1-oxo-isoindolin-2-yl]piperidine-2,6-dione (3.58 g, 6.64 mmol) at 20° C. under air. The resulting solution was stirred at 50° C. for 2.5 hours. The reaction mixture was evaporated to dryness, DCM added (50 ml), evaporated to dryness again and dissolved in IPA (20 mL) and DCM (40 mL). Sodium triacetoxyborohydride (3.52 g, 16.6 mmol) was added and the mixture stirred for 30 min. The reaction mixture was diluted with DCM (170 mL) and saturated NaHCO₃ (170 mL), the layers were separated, and the aqueous layer was extracted with DCM (100 mL). The combined organic layers were dried with MgSO₄, filtered and evaporated to afford crude product. The residue was dissolved in DCM, absorbed on to alumina and evaporated to dryness. The residue was purified by flash amino-silica chromatography, elution gradient 0 to 2% MeOH in DCM. Pure fractions were evaporated to dryness. The residue was dissolved in 18 mL of DMSO/IPA (1:1) and purified on Sepiatec SFC system using the following SFC conditions: Column: Thar 2-EP 30×250 mm, 5 micron Mobile phase: A=2-propanol+0.1% DEA/B=scCO2 Gradient 35-45% A over 5 minutes; Flow rate: 90 ml/min; BPR: 120 bar; Temperature: 40 deg C.; 210 nm. The product containing fractions were evaporated to dryness, redissolved in MeCN, sonicated for 5 min and evaporated to dryness to afford the title compound (1.65 g, 1.92 mmol, 35%) as a white solid; ¹H NMR (400 MHz, DMSO, 30° C.) 0.99 (2H, q), 1.09 (3H, d), 1.17 (2H, d), 1.28 (3H, d), 1.42 (3H, d), 1.75 (6H, t), 1.89-2.02 (1H, m), 2.13 (4H, t), 2.22-2.49 (11H, m), 2.53-2.66 (2H, m), 2.68-3 (6H, m), 3.15 (1H, s), 3.86 (2H, d), 4.20 (1H, d), 4.32 (1H, d), 4.61 (2H, d), 4.91 (1H, s), 5.04 (1H, dd), 6.93-7.1 (4H, m), 7.28 (1H, d), 7.43 (1H, d), 7.50 (1H, d), 8.10 (2H, s), 10.70 (1H, s), 10.91 (1H, s); m/z: ES+ [M+H]+ 859.6.

### Intermediate 7a: tert-butyl 9-[2-(2,6-dioxo-3-piperidyl)-1-oxo-isoindolin-5-yl]-3,9-diazaspiro[5.5]undecane-3-carboxylate

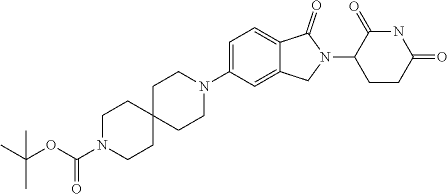 [SMILES: CC(C)(C)OC(=O)N1CCC2(CC1)CCN(c1ccc3c(c1)CN(C1CCC(=O)NC1=O)C3=O)CC2]

Pd-PEPPSI-IHept^{Cl} (0.53 g, 0.54 mmol) was added to a degassed mixture of 3-(5-Bromo-1-oxoisoindolin-2-yl)piperidine-2,6-dione (3.5 g, 10.8 mmol), 3-Boc-3,9-diazaspiro[5.5]undecane (3.6 g, 14.2 mmol) and cesium carbonate (10.6 g, 32.5 mmol) in 1,4-dioxane (100 mL) at 40° C. under nitrogen. The resulting mixture was vacuum degassed, backfilling with nitrogen and stirred at 100° C. for 3 hours. The reaction mixture was diluted with DCM (150 mL) and 10% aq. AcOH (100 mL), the layers were separated, and the aqueous layer was extracted with DCM (150 mL). The combined organic layers were dried with MgSO₄, filtered and evaporated to afford crude product. The crude solid was triturated with EtOAc (35 mL) to give a solid which was collected by filtration, washed sequentially with EtOAc (2×15 mL) and EtOAc:Et₂O (1:1; 20 mL) and dried under vacuum to give the title compound (4.14 g, 77%) as a pale blue solid; ¹H NMR (400 MHz, DMSO, 30° C.) 1.40 (13H, s), 1.51-1.64 (4H, m), 1.96 (1H, ddd), 2.27-2.44 (1H, m), 2.59 (1H, dt), 2.90 (1H, ddd), 3.32 (8H, dd), 4.20 (1H, d), 4.32 (1H, d), 5.04 (1H, dd), 7.04 (2H, d), 7.50 (1H, d), 10.91 (1H, s); m/z: ES+ [M+H]+ 497.3.

### Intermediate 7b: (1R,3R)-1-(2-(4-(Dibutoxymethyl)piperidin-1-yl)pyrimidin-5-yl)-2-(2-fluoro-2-methylpropyl)-3-methyl-2,3,4,9-tetrahydro-1H-pyrido[3,4-b]indole

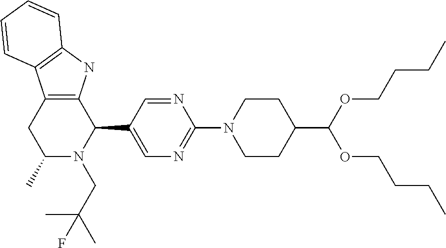 [SMILES: CCCCOC(OCCCC)C1CCN(c2ncc([C@@H]3c4[nH]c5ccccc5c4C[C@@H](C)N3CC(C)(C)F)cn2)CC1]

4-(Dibutoxymethyl)piperidine (2.15 g, 8.85 mmol), DIPEA (4.30 ml, 24.1 mmol) and (1R,3R)-1-(2-chloropyrimidin-5-yl)-2-(2-fluoro-2-methylpropyl)-3-methyl-2,3,4,9-tetrahydro-1H-pyrido[3,4-b]indole (3 g, 8.05 mmol) were dissolved in DMSO (25 mL) and heated to 90° C. for 2 hours and 30 minutes and cooled to RT. The reaction mixture was diluted with ethyl acetate (500 mL) and washed sequentially with water (3×100 mL) and brine (100 mL). The organic phase was dried over MgSO₄, filtered and concentrated. The crude product was purified by flash silica chromatography, elution gradient 0 to 100% EtOAc in heptane. Pure fractions were evaporated to dryness to afford the title compound (4.00 g, 86%) as a colourless gum; ¹H NMR (400 MHz, CDCl₃, 30° C.) 0.85-0.96 (6H, m), 1.09 (3H, d), 1.21-1.42 (11H, m), 1.46 (3H, d), 1.51-1.6 (2H, m), 1.79-1.94 (3H, m), 2.46-2.75 (4H, m), 2.75-2.89 (2H, m), 3.18-3.35 (1H, m), 3.42 (2H, dt), 3.61 (2H, dt), 4.14 (1H, d), 4.75 (2H, d), 4.99 (1H, s), 7.11 (1H, td), 7.16 (1H, td), 7.26-7.3 (1H, m), 7.51 (1H, d), 7.63 (1H, s), 8.17 (2H, s); m/z: ES+ [M+H]+ 581.5

### Example 7: 3-(5-{9-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]-3,9-diazaspiro[5.5]undecan-3-yl}-1-oxo-1,3-dihydro-2H-isoindol-2-yl)piperidine-2,6-dione

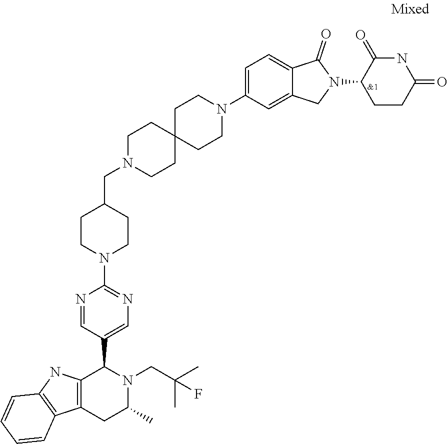 [SMILES: C[C@@H]1Cc2c([nH]c3ccccc23)[C@@H](c2cnc(N3CCC(CN4CCC5(CC4)CCN(c4ccc6c(c4)CN([C@H]4CCC(=O)NC4=O)C6=O)CC5)CC3)nc2)N1CC(C)(C)F]

Formic acid (40 mL) was added to (1R,3R)-1-(2-(4-(Dibutoxymethyl)piperidin-1-yl)pyrimidin-5-yl)-2-(2-fluoro-2-methylpropyl)-3-methyl-2,3,4,9-tetrahydro-1H-pyrido[3,4-b]indole (3.5 g, 6.04 mmol) and tert-butyl 9-[2-(2,6-dioxo-3-piperidyl)-1-oxo-isoindolin-5-yl]-3,9-diazaspiro[5.5]undecane-3-carboxylate (3.60 g, 7.24 mmol) at 20° C. under air. The resulting solution was stirred at 50° C. for 2.5 hours. The reaction mixture was evaporated to dryness, DCM added (50 mL), evaporated to dryness again and dissolved in IPA (20 mL) and DCM (40 mL). sodium triacetoxyborohydride (3.84 g, 18.1 mmol) was added and the mixture stirred for 30 min (mild effervescence). The reaction mixture was diluted with DCM (170 mL) and saturated NaHCO₃ (170 mL), the layers were separated, and the aqueous layer was extracted with DCM (170 mL). The combined organic layers were dried with MgSO₄, filtered and evaporated to afford crude product. The residue was dissolved in DCM, absorbed on to alumina and evaporated to dryness. The crude product was purified by flash amino-silica chromatography, elution gradient 0 to 2.5% MeOH in DCM. The product containing fractions were evaporated to dryness. The residue was dissolved in 10.0 ml of DMSO/IPA 1:1 and purified on Sepiatec SFC system using the following SFC conditions: Column: Thar 2-EP 30×250 mm, 5 micron Mobile phase: A=2-propanol+0.1% DEA/B=scCO2 Gradient 35-45% A over 5 minutes; Flow rate: 90 ml/min BPR: 120 bar Temperature: 40 deg C. UV max 210 nm. The product containing fractions were evaporated to dryness, suspended in MeCN (50 mL), sonicated for 5 min and evaporated to dryness to afford the title compound (2.1 g, 42%) as a white solid; ¹H NMR (400 MHz, DMSO, 30° C.) 0.89-1.06 (2H, m), 1.09 (3H, d), 1.28 (3H, d), 1.35-1.59 (11H, m), 1.66-1.87 (3H, m), 1.9-2.01 (1H, m), 2.14 (2H, d), 2.27-2.49 (7H, m), 2.54-2.65 (2H, m), 2.68-2.98 (4H, m), 3.15 (1H, s), 3.30 (4H, s), 4.20 (1H, d), 4.32 (1H, d), 4.61 (2H, d), 4.91 (1H, s), 5.04 (1H, dd), 6.98 (1H, td), 7.01-7.1 (3H, m), 7.28 (1H, d), 7.43 (1H, d), 7.50 (1H, d), 8.10 (2H, s), 10.71 (1H, s), 10.91 (1H, s); m/z: ES+ [M+H]+ 830.6.

### Intermediate 8a: 3-(1-(5-((1R,3R)-2-(2-fluoro-2-methylpropyl)-3-methyl-2,3,4,9-tetrahydro-1H-pyrido[3,4-b]indol-1-yl)pyrimidin-2-yl)piperidin-4-yl)propan-1-ol

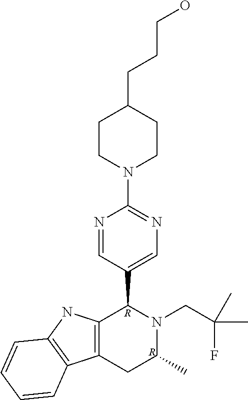

3-(Piperidin-4-yl)propan-1-ol (230 mg, 1.61 mmol), DIPEA (0.70 mL, 4.02 mmol) and (1R,3R)-1-(2-chloropyrimidin-5-yl)-2-(2-fluoro-2-methylpropyl)-3-methyl-2,3,4,9-tetrahydro-1H-pyrido[3,4-b]indole (500 mg, 1.34 mmol) were dissolved in DMF (3.8 mL) and sealed into a microwave tube. The reaction was heated to 140° C. for 30 minutes in the microwave reactor. The reaction mixture was diluted with methanol (1 mL) and was purified by preparative HPLC (Waters XSelect CSH C18 ODB column, 5p silica, 30 mm diameter, 100 mm length), using decreasingly polar mixtures of water (containing 1% by volume NH₃OH (28-30% in H₂O)) and MeCN as eluents to afford the title compound (400 mg, 62%) as a light pink foam; ¹H NMR (400 MHz, CDCl₃, 30° C.) 1.06-1.22 (5H, m), 1.24-1.37 (5H, m), 1.41-1.66 (7H, m), 1.76 (2H, d), 2.43-2.76 (4H, m), 2.84 (2H, t), 3.18-3.33 (1H, m), 3.64 (2H, q), 4.71 (2H, d), 4.97 (1H, s), 7.13 (2H, dtd), 7.27 (1H, d), 7.51 (1H, d), 7.81 (1H, s), 8.16 (2H, s); m/z: ES+ [M+H]+ 480.3.

### Intermediate 8b: 3-(1-(5-((1R,3R)-2-(2-fluoro-2-methylpropyl)-3-methyl-2,3,4,9-tetrahydro-1H-pyrido[3,4-b]indol-1-yl)pyrimidin-2-yl)piperidin-4-yl)propanal

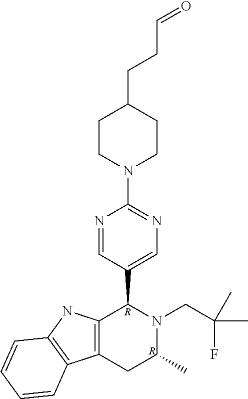

SO₃-pyridine complex (143 mg, 0.90 mmol) was added to a solution of 3-(1-(5-((1R,3R)-2-(2-fluoro-2-methylpropyl)-3-methyl-2,3,4,9-tetrahydro-1H-pyrido[3,4-b]indol-1-yl)pyrimidin-2-yl)piperidin-4-yl)propan-1-ol (215 mg, 0.45 mmol) and triethylamine (0.125 mL, 0.90 mmol) in DCM (1 mL)-DMSO (1 mL) at 20° C. The reaction was allowed to warm to rt for 1 hour. The reaction was diluted with DCM (20 mL) and water (20 mL), the layers were separated. The organic was washed with brine (20 mL), dried over a phase separator and evaporated to afford crude product that was used in the next step without further purification; m/z: ES+ [M+H]+ 540.3.

### Example 8: 3-(5-{4-[3-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)propyl]piperazin-1-yl}-1-oxo-1,3-dihydro-2H-isoindol-2-yl)piperidine-2,6-dione

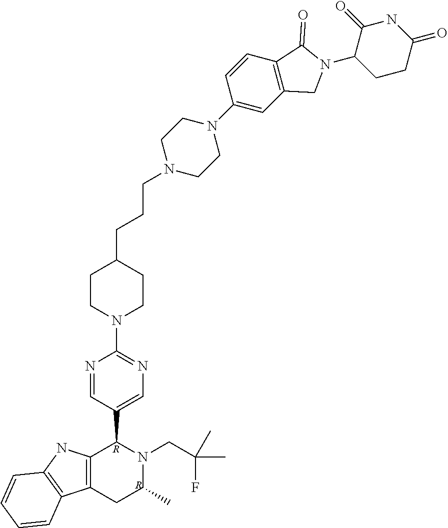

A solution of 3-(1-oxo-5-(piperazin-1-yl)isoindolin-2-yl)piperidine-2,6-dione, HCl (185 mg, 0.46 mmol), 3-(1-(5-((1R,3R)-2-(2-fluoro-2-methylpropyl)-3-methyl-2,3,4,9-tetrahydro-1H-pyrido[3,4-b]indol-1-yl)pyrimidin-2-yl)piperidin-4-yl)propanal (200 mg, 0.42 mmol) and sodium acetate (103 mg, 1.26 mmol) in DCM (2.8 mL) and MeOH (1.4 mL) was stirred at rt under nitrogen for 45 minutes. Sodium cyanotrihydroborate (79 mg, 1.26 mmol) was added and the resulting solution was stirred at rt for 1 hour. The reaction mixture was diluted with methanol (2 mL), filtered and purified by preparative HPLC (Waters CSH C18 OBD column, 30×100 mm id, 5 micron particle size), using decreasingly polar mixtures of water (containing 0.1% formic acid) and MeCN as eluents. Fractions containing the desired compound were saturated with sodium chloride and extracted with chloroform (3×30 mL). The combined organic phase were dried over a phase separator and concentrated. The crude product was purified by flash silica chromatography, elution gradient 0 to 10% EtOH in EtOAc to afford the title compound (61 mg, 18%) as a white solid; ¹H NMR (500 MHz, CDCl₃, 27° C.) 1.12 (3H, d), 1.14-1.23 (2H, m), 1.26-1.36 (5H, m), 1.39-1.71 (6H, m), 1.79 (2H, d), 2.11-2.76 (12H, m), 2.77-2.98 (4H, m), 3.17-3.51 (5H, m), 4.28 (1H, d), 4.43 (1H, d), 4.74 (2H, d), 5.02 (1H, s), 5.21 (1H, dd), 6.90 (1H, s), 7.01 (1H, d), 7.11-7.16 (1H, m), 7.16-7.21 (1H, m), 7.30 (1H, d), 7.48-7.58 (1H, m), 7.68-7.83 (2H, m), 7.98 (1H, s), 8.20 (2H, s); m/z: ES+ [M+H]+ 790.4.

### Intermediate 9a: tert-Butyl 9-(5-((1R,3R)-2-(2-fluoro-2-methylpropyl)-3-methyl-2,3,4,9-tetrahydro-1H-pyrido[3,4-b]indol-1-yl)pyrimidin-2-yl)-3,9-diazaspiro[5.5]undecane-3-carboxylate

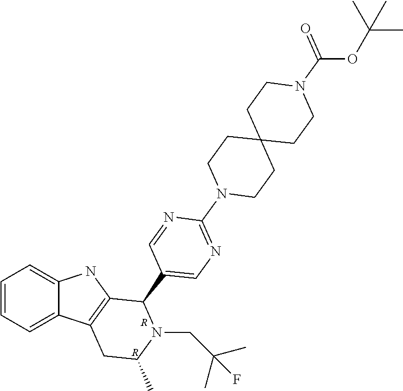

tert-Butyl 3,9-diazaspiro[5.5]undecane-3-carboxylate hydrochloride (0.772 g, 2.66 mmol), (1R,3R)-1-(2-chloropyrimidin-5-yl)-2-(2-fluoro-2-methylpropyl)-3-methyl-2,3,4,9-tetrahydro-1H-pyrido[3,4-b]indole (0.9 g, 2.41 mmol) and DIPEA (1.26 mL, 7.24 mmol) were stirred in DMF (10 mL) under nitrogen and the mixture was heated to 90° C. for 3 hours. The mixture was partitioned between ethyl acetate (100 mL) and saturated sodium bicarbonate solution (100 mL). The organic phase was dried over MgSO₄, filtered, evaporated then purified by flash silica chromatography, elution gradient 0 to 100% EtOAc in heptane to afford the title compound (1.05 g, 74%) as a white solid; ¹H NMR (400 MHz, CDCl₃, 30° C.) 1.09 (3H, d), 1.29 (6H, d), 1.42-1.55 (17H, m), 2.44-2.63 (2H, m), 2.67 (2H, d), 3.25 (1H, s), 3.34-3.44 (4H, m), 3.68-3.84 (4H, m), 4.99 (1H, s), 7.10 (1H, td), 7.16 (1H, td), 7.26-7.3 (1H, m), 7.51 (1H, d), 7.84 (1H, s), 8.17 (2H, s); m/z: ES− [M−H]⁻ 589.1.

### Example 9: 3-(5-{4-[(9-{5-[(1R,3R)-2-(2-fluoro-2-methylpropyl)-3-methyl-2,3,4,9-tetrahydro-1H-beta-carbolin-1-yl]pyrimidin-2-yl}-3,9-diazaspiro[5.5]undecan-3-yl)methyl]piperidin-1-yl}-1-oxo-1,3-dihydro-2H-isoindol-2-yl)piperidine-2,6-dione

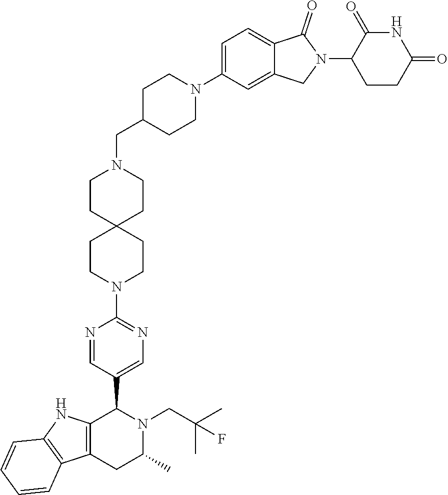 [SMILES: C[C@@H]1Cc2c([nH]c3ccccc23)[C@@H](c2cnc(N3CCC4(CCN(CC5CCN(c6ccc7c(c6)CN(C6CCC(=O)NC6=O)C7=O)CC5)CC4)CC3)nc2)N1CC(C)(C)F]

Formic acid (40 mL) was added to tert-Butyl 9-(5-((1R,3R)-2-(2-fluoro-2-methylpropyl)-3-methyl-2,3,4,9-tetrahydro-1H-pyrido[3,4-b]indol-1-yl)pyrimidin-2-yl)-3,9-diazaspiro[5.5]undecane-3-carboxylate (2.5 g, 4.23 mmol) and 3-[5-[4-(dibutoxymethyl)-1-piperidyl]-1-oxo-isoindolin-2-yl]piperidine-2,6-dione (2.4 g, 4.45 mmol) at 20° C. under air. The resulting solution was stirred at 50° C. for 1.5 hours. The reaction mixture was evaporated to dryness, DCM added (50 ml), evaporated to dryness again and dissolved in IPA (20 mL) and DCM (40 mL). Sodium triacetoxyborohydride (2.7 g, 12.74 mmol) was added and the mixture stirred for 30 min. The reaction mixture was diluted with DCM (170 mL) and saturated NaHCO₃ (170 mL), the layers were separated, and the aqueous layer was extracted with DCM (100 mL). The combined organic layers were dried with MgSO₄, filtered and evaporated to afford crude product. The residue was dissolved in DCM, absorbed on to alumina and evaporated to dryness. The crude product was purified by flash amino-silica chromatography, elution gradient 0 to 2% MeOH in DCM. The product containing fractions were evaporated to afford the title compound (3.35 g, 95%) as a white solid; ¹H NMR (400 MHz, DMSO, 30° C.) 1.08 (3H, d), 1.11-1.22 (2H, m), 1.28 (3H, d), 1.34-1.55 (11H, m), 1.77 (3H, d), 1.85-2.01 (1H, m), 2.15 (2H, d), 2.27-2.44 (5H, m), 2.44-2.49 (1H, m), 2.52-2.64 (3H, m), 2.67-2.98 (4H, m), 3.14 (1H, s), 3.63-3.75 (4H, m), 3.85 (2H, d), 4.20 (1H, d), 4.32 (1H, d), 4.92 (1H, s), 5.04 (1H, dd), 6.92-7.11 (4H, m), 7.28 (1H, d), 7.43 (1H, d), 7.50 (1H, d), 8.10 (2H, s), 10.71 (1H, s), 10.91 (1H, s); m/z: ES+ [M+H]⁺ 830.5.

### Intermediate 10a: 3-(5-(4-(2,2-dimethoxyethyl)piperidin-1-yl)-1-oxoisoindolin-2-yl)piperidine-2,6-dione

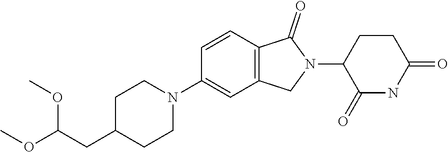 [SMILES: COC(CC1CCN(c2ccc3c(c2)CN(C2CCC(=O)NC2=O)C3=O)CC1)OC]

Pd-PEPPSI-IHept^{Cl} (0.602 g, 0.62 mmol) was added to 3-(5-bromo-1-oxoisoindolin-2-yl)piperidine-2,6-dione (4.0 g, 12 mmol), cesium carbonate (12.1 g, 37.1 mmol) and 4-(2,2-dimethoxyethyl)piperidine (2.25 g, 13.0 mmol) in 1,4-dioxane (45 mL) at 20° C. under nitrogen. The resulting suspension was stirred at 105° C. for 2 hours. The reaction mixture was diluted with DCM (200 mL), and washed sequentially with 5% AcOH in water (100 mL) and saturated brine (100 mL). The organic layer was dried with MgSO₄, filtered and evaporated to afford crude dark blue product. The crude powder was triturated with EtOAc (30 mL) to give a solid which was collected by filtration, washed with EtOAc: ether (1:1; 30 mL) and dried under vacuum to give the title compound (3.90 g, 76%) as a grey powder; ¹H NMR (400 MHz, DMSO, 30° C.) 1.25 (2H, qd), 1.49 (2H, t), 1.53-1.68 (1H, m), 1.76 (2H, d), 1.97 (1H, ddq), 2.29-2.43 (1H, m), 2.54-2.64 (1H, m), 2.75-2.85 (2H, m), 2.90 (1H, ddd), 3.23 (6H, s), 3.85 (2H, d), 4.20 (1H, d), 4.32 (1H, d), 4.48 (1H, t), 5.04 (1H, dd), 7.03 (2H, d), 7.45-7.54 (1H, m), 10.91 (1H, s); m/z: ES+ [M+H]+ 416.3.

### Example 10: 3-(5-{4-[2-(9-{5-[(1R,3R)-2-(2-fluoro-2-methylpropyl)-3-methyl-2,3,4,9-tetrahydro-1H-beta-carbolin-1-yl]pyrimidin-2-yl}-3,9-diazaspiro[5.5]undecan-3-yl)ethyl]piperidin-1-yl}-1-oxo-1,3-dihydro-2H-isoindol-2-yl)piperidine-2,6-dione

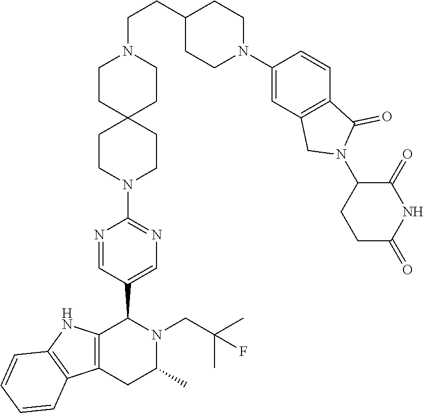 [SMILES: C[C@@H]1Cc2c([nH]c3ccccc23)[C@@H](c2cnc(N3CCC4(CCN(CCC5CCN(c6ccc7c(c6)CN(C6CCC(=O)NC6=O)C7=O)CC5)CC4)CC3)nc2)N1CC(C)(C)F]

Formic acid (3 mL) was added to tert-butyl 9-(5-((1R,3R)-2-(2-fluoro-2-methylpropyl)-3-methyl-2,3,4,9-tetrahydro-1H-pyrido[3,4-b]indol-1-yl)pyrimidin-2-yl)-3,9-diazaspiro[5.5]undecane-3-carboxylate (60 mg, 0.10 mmol) and 3-(5-(4-(2,2-dimethoxyethyl)piperidin-1-yl)-1-oxoisoindolin-2-yl)piperidine-2,6-dione (50 mg, 0.12 mmol) at rt under air. The resulting solution was stirred at 40° C. for 1 hour. The resulting mixture was evaporated to dryness. The mixture was redissolved in DCM (2 mL) and IPA (1 mL) and sodium triacetoxyborohydride (60 mg, 0.28 mmol) added at 20° C. The resulting suspension was stirred for 30 minutes under air at rt. The reaction was incomplete and further sodium triacetoxyborohydride (60 mg, 0.28 mmol) was added and the suspension was stirred at 20° C. for a further 30 minutes. The reaction mixture was diluted with DCM (20 mL), water (10 mL) and sat. aq. NaHCO₃ (10 mL), the layers were separated, and the aqueous layer was extracted with (DCM) (3×20 mL). The combined organic layers were dried with MgSO₄, filtered and evaporated. The crude product was purified by preparative HPLC (Waters XSelect CSH C18 column, 5p silica, 30 mm diameter, 100 mm length), using decreasingly polar mixtures of water (containing 1% formic acid) and MeCN as eluents. Fractions containing the desired compound were partially evaporated to remove the MeCN, basified with sat. aq. NaHCO₃ to pH 8 and extracted with DCM (3×20 mL) The combined organic portions were dried over MgSO₄ and evaporated to dryness to afford impure product. The solid was further purified by flash amino-silica chromatography, elution gradient 0 to 5% MeOH in DCM to afford the title compound (34 mg, 40%) as a white solid; ¹H NMR (400 MHz, DMSO, 30° C.) 1.08 (3H, d), 1.19-1.33 (5H, m), 1.33-1.56 (14H, m), 1.74 (2H, d), 1.91-1.99 (1H, m), 2.26-2.42 (7H, m), 2.44-2.48 (1H, m), 2.54-2.7 (3H, m), 2.71-2.85 (3H, m), 2.90 (1H, ddd), 3.14 (1H, s), 3.58-3.75 (4H, m), 3.85 (2H, d), 4.20 (1H, d), 4.32 (1H, d), 4.92 (1H, s), 5.04 (1H, dd), 6.94-7.09 (4H, m), 7.28 (1H, d), 7.43 (1H, d), 7.49 (1H, d), 8.10 (2H, s), 10.71 (1H, s), 10.92 (1H, s); m z: ES+ [M+H]+ 844.6.

### Example 11: 3-(5-{9-[2-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl]-3,9-diazaspiro[5.5]undecan-3-yl}-1-oxo-1,3-dihydro-2H-isoindol-2-yl)piperidine-2,6-dione

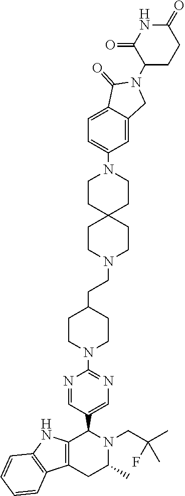 [SMILES: C[C@@H]1Cc2c([nH]c3ccccc23)[C@@H](c2cnc(N3CCC(CCN4CCC5(CC4)CCN(c4ccc6c(c4)CN(C4CCC(=O)NC4=O)C6=O)CC5)CC3)nc2)N1CC(C)(C)F]

A slurry of 3-(1-oxo-5-(3,9-diazaspiro[5.5]undecan-3-yl)isoindolin-2-yl)piperidine-2,6-dione, HCl (392 mg, 0.91 mmol), 2-(1-(5-((1R,3R)-2-(2-fluoro-2-methylpropyl)-3-methyl-2,3,4,9-tetrahydro-1H-pyrido[3,4-b]indol-1-yl)pyrimidin-2-yl)piperidin-4-yl)acetaldehyde (350 mg, 0.75 mmol) in DCM (1 mL) and 2-propanol (1 mL) was stirred at room temperature under nitrogen for 15 minutes. Sodium triacetoxyhydroborate (480 mg, 2.26 mmol) was added portionwise and the resulting solution was stirred at RT for 2 days. The reaction mixture was evaporated, diluted with DCM (20 mL) and water (20 mL). The layers were separated and the aqueous phase extracted with DCM (2×20 mL). The combined organic phases were washed with brine (20 mL). The organics were dried over a phase separator and concentrated. The crude product was purified by preparative HPLC (Waters XSelect CSH C18 ODB column, 5p silica, 30 mm diameter, 100 mm length), using decreasingly polar mixtures of water (containing 0.1% formic acid) and MeCN as eluents. Fractions containing the desired compound were combined, evaporated cold to a minimum amount of solvent, basified with saturated NaHCO₃. The aqueous phase was extracted with DCM (4×30 mL). The combined organic phases were washed with water (20 mL), dried over a phase separator and evaporated cold to dryness to afford the title compound (44 mg, 7%) as a pale beige solid; ¹H NMR (400 MHz, CDCl₃, 30° C.) 1.10 (3H, d), 1.14-1.36 (7H, m), 1.41-1.67 (12H, m), 1.76 (2H, d), 2.00 (0H, s), 2.15-2.23 (1H, m), 2.25-2.46 (7H, m), 2.48-2.74 (4H, m), 2.76-3.01 (4H, m), 3.14-3.39 (5H, m), 4.24 (1H, d), 4.40 (1H, d), 4.70 (2H, d), 4.99 (1H, s), 5.19 (1H, dd), 5.30 (0H, s), 6.86 (1H, s), 6.97 (1H, dd), 7.11 (1H, td), 7.16 (1H, td), 7.26-7.29 (1H, m), 7.49-7.55 (1H, m), 7.56-7.67 (1H, m), 7.71 (1H, d), 7.76-8 (1H, m), 8.17 (2H, s), 8.30 (0H, s); m/z: ES− [M−H]⁻ 842.1.

### Intermediate 12a: 5-Fluoro-7-methoxyisobenzofuran-1(3H)-one

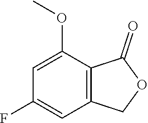

Palladium(II) acetate (1.06 g, 4.7 mmol) was added in one portion to 4-fluoro-2-methoxybenzoic acid (8 g, 47 mmol), dibromomethane (10 mL, 143 mmol) and potassium phosphate, dibasic (24.57 g, 141 mmol) in dioxane (5 mL) at 25° C. under nitrogen. The resulting solution was stirred at 140° C. for 3 days. The reaction mixture was filtered through celite. The filtrate was concentrated and purified by flash silica chromatography, elution gradient 0 to 20% EtOAc in petroleum ether to afford the title compound (3.52 g, 41%) as a white solid; ¹H NMR (400 MHz, CDCl₃, 24° C.) 4.00 (3H, s), 5.23 (2H, s), 6.63-6.77 (2H, m); m/z: ES+ [M+H]+ 183.1.

### Intermediate 12b: tert-Butyl 4-(7-methoxy-1-oxo-1,3-dihydroisobenzofuran-5-yl)piperazine-1-carboxylate

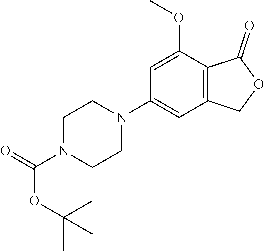 [SMILES: COc1cc(N2CCN(C(=O)OC(C)(C)C)CC2)cc2c1C(=O)OC2]

tert-Butyl piperazine-1-carboxylate (4.65 g, 25.0 mmol) was added to 5-fluoro-7-methoxyisobenzofuran-1(3H)-one (3.5 g, 19 mmol) in DMSO (30 mL). The resulting solution was stirred at 120° C. for 50 hours. The reaction mixture was diluted with water (150 mL), filtered. The filter cake was washed with water (3×25 mL), concentrated and purified by flash silica chromatography, elution gradient 0 to 60% EtOAc in DCM to afford the title compound (4.30 g, 64%) as a white solid; ¹H NMR (300 MHz, DMSO, 24° C.) 1.43 (9H, s), 3.36-3.52 (8H, m), 3.87 (3H, s), 5.13 (2H, s), 6.48 (1H, d), 6.57 (1H, d); m/z: ES+ [M+H]+ 349.1.

### Intermediate 12c: 4-(4-(tert-butoxycarbonyl)piperazin-1-yl)-2-(hydroxymethyl)-6-methoxybenzoic acid

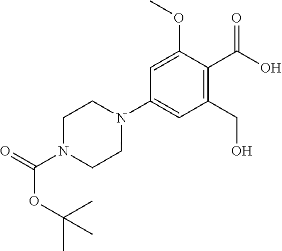 [SMILES: COc1cc(N2CCN(C(=O)OC(C)(C)C)CC2)cc(CO)c1C(=O)O]

Sodium hydroxide (0.046 g, 1.15 mmol) was added to tert-butyl 4-(7-methoxy-1-oxo-1,3-dihydroisobenzofuran-5-yl)piperazine-1-carboxylate (0.1 g, 0.29 mmol) in MeOH (40 mL), THF (40 mL) and water (40 mL). The resulting solution was stirred at RT for 4 hours. The reaction mixture was diluted with water (100 mL) and washed sequentially with EtOAc (4×200 mL) and saturated brine (2×100 mL), The organic layer was dried over MgSO₄, filtered and evaporated to afford the title compound (5.1 g, 97%) as a white solid that was used in the next step directly without further purification; ¹H NMR (300 MHz, DMSO, 24° C.) 1.43 (9H, s), 3.10-3.28 (4H, m), 3.30-3.55 (4H, m), 3.76 (3H, s), 4.45 (2H, s), 5.11 (1H, s), 6.47 (1H, d), 6.66 (1H, d), 12.40 (1H, s); m/z: ES+ [M+H]+ 367.1.

### Intermediate 12d: tert-Butyl 4-(3-(hydroxymethyl)-5-methoxy-4-(methoxycarbonyl)phenyl)piperazine-1-carboxylate

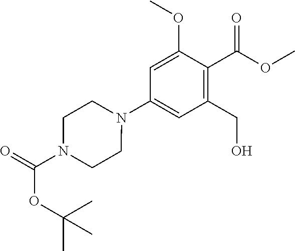 [SMILES: COC(=O)c1c(CO)cc(N2CCN(C(=O)OC(C)(C)C)CC2)cc1OC]

Trimethylsilyl-diazomethane (20.47 mL, 40.94 mmol) was added dropwise to 4-(4-(tert-butoxycarbonyl)piperazin-1-yl)-2-(hydroxymethyl)-6-methoxybenzoic acid (5 g, 13.65 mmol) in MeOH (40 mL) and EtOAc (40 mL) at −10° C. The resulting solution was stirred at −10° C. for 2 hours. The reaction mixture was quenched with water (100 mL), extracted with EtOAc (3×300 mL), the organic layer was dried over MgSO₄, filtered and evaporated to afford the title compound (4.0 g, 77%) as a white solid that was used in the next step directly without further purification; ¹H NMR (300 MHz, DMSO, 24° C.) 1.43 (9H, s), 3.18-3.34 (4H, m), 3.36-3.55 (4H, m), 3.65-3.80 (6H, m), 4.32 (1H, s), 4.60 (2H, s), 6.54 (1H, d), 6.68 (1H, d); m/z: ES+ [M+H]+ 381.1.

### Intermediate 12e: tert-Butyl 4-(3-(bromomethyl)-5-methoxy-4-(methoxycarbonyl)phenyl)piperazine-1-carboxylate

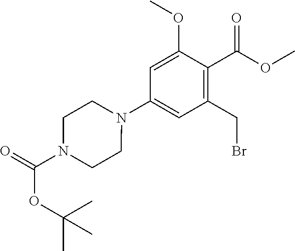 [SMILES: COC(=O)c1c(CBr)cc(N2CCN(C(=O)OC(C)(C)C)CC2)cc1OC]

Triphenylphosphine (3.59 g, 13.7 mmol) was added in one portion to tert-butyl 4-(3-(hydroxymethyl)-5-methoxy-4-(methoxycarbonyl)phenyl)piperazine-1-carboxylate (4.00 g, 10.5 mmol) and carbon tetrabromide (4.53 g, 13.7 mmol) in THF (80 mL) at 25° C. The resulting solution was stirred at 25° C. for 16 hours. The reaction mixture was filtered and the filtrate was concentrated and purified by flash silica chromatography, elution gradient 0 to 8% EtOAc in petroleum ether to afford the title compound (2.5 g, 54%) as a white solid; ¹H NMR (300 MHz, DMSO, 24° C.) 1.43 (9H, s), 3.20-3.32 (4H, m), 3.41-3.48 (4H, m), 3.65-4.00 (6H, m), 4.60 (2H, s), 6.54 (1H, d), 6.68 (1H, d); m/z: ES+ [M+H]+ 443.0.

### Intermediate 12f: tert-Butyl 4-(2-(2,6-dioxopiperidin-3-yl)-7-methoxy-1-oxoisoindolin-5-yl)piperazine-1-carboxylate

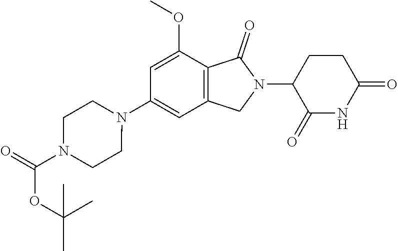 [SMILES: COc1cc(N2CCN(C(=O)OC(C)(C)C)CC2)cc2c1C(=O)N(C1CCC(=O)NC1=O)C2]

DIPEA (2.95 mL, 16.9 mmol) was added in one portion to tert-butyl 4-(3-(bromomethyl)-5-methoxy-4-(methoxycarbonyl)phenyl)piperazine-1-carboxylate (2.5 g, 5.64 mmol) and 3-aminopiperidine-2,6-dione hydrochloride (1.39 g, 8.46 mmol) in acetonitrile (2 mL) at 25° C. The resulting solution was stirred at 80° C. for 16 hours. The reaction mixture was filtered through a glass fiber paper and the cake washed with THF (3×20 mL). The filtrate was concentrated and purified by flash silica chromatography, elution gradient 0 to 50% EtOAc in DCM to afford the title compound (1.43 g, 55%) as a white solid; ¹H NMR (300 MHz, CDCl₃, 24° C.) 1.51 (9H, s), 2.11-2.25 (1H, m), 2.25-2.43 (1H, m), 2.74-2.96 (2H, m), 3.25-3.40 (4H, m), 3.60-3.72 (4H, m), 3.97 (3H, s), 4.23 (1H, d), 4.39 (1H, d), 5.10-5.22 (1H, m), 6.47 (1H, s), 6.55 (1H, s), 8.03 (1H, s); m/z (ES+), [M+H]+=459.1.

### Intermediate 122: 3-(7-Methoxy-1-oxo-5-(piperazin-1-yl)isoindolin-2-yl)piperidine-2,6-dione, bis formate salt

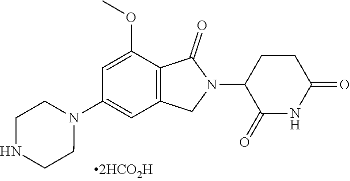 [SMILES: COc1cc(N2CCNCC2)cc2c1C(=O)N(C1CCC(=O)NC1=O)C2]

Formic acid (1.43 g, 31.2 mmol) was added to tert-butyl 4-(2-(2,6-dioxopiperidin-3-yl)-7-methoxy-1-oxoisoindolin-5-yl)piperazine-1-carboxylate (1.43 g, 3.12 mmol). The resulting solution was stirred at RT for 3 hours. The reaction mixture was concentrated and the crude product was purified by flash C18-flash chromatography, elution gradient 0 to 30% MeCN in water (0.1% formic acid) to afford the title compound (1.30 g, 97%) as a black solid that was used in the next step without further purification; ¹H NMR (300 MHz, DMSO, 24° C.) 1.85-1.99 (1H, m), 2.22-2.42 (1H, m), 2.51-2.65 (1H, m), 2.80-2.98 (1H, m), 3.10-3.26 (2H, m), 3.27-3.39 (1H, m), 3.40-3.57 (4H, m), 3.84 (3H, s), 4.06-4.18 (1H, m), 4.19-4.31 (1H, m), 4.91-5.03 (1H, m), 6.51-6.59 (1H, m), 6.61-6.69 (1H, m), 6.78-7.19 (4H, m), 8.19 (2H, d), 10.95 (1H, s); m/z: ES+ [M+H]+ 359.1.

### Example 12: 3-(5-{4-[2-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl]piperazin-1-yl}-7-methoxy-1-oxo-1,3-dihydro-2H-isoindol-2-yl)piperidine-2,6-dione

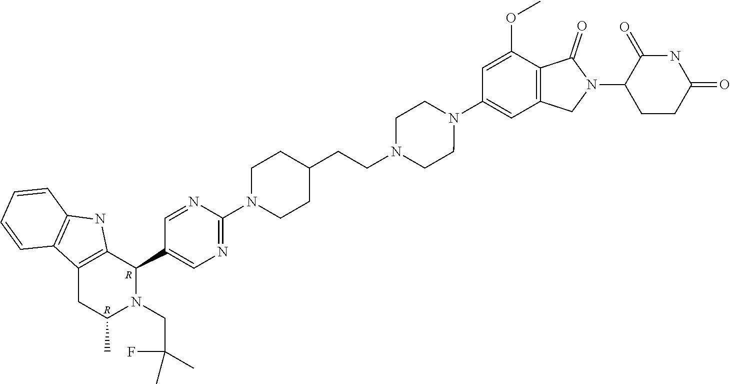

A solution of crude 3-(7-methoxy-1-oxo-5-(piperazin-1-yl)isoindolin-2-yl)piperidine-2,6-dione, bis formate salt (190 mg, 0.42 mmol), 2-(1-(5-((1R,3R)-2-(2-fluoro-2-methylpropyl)-3-methyl-2,3,4,9-tetrahydro-1H-pyrido[3,4-b]indol-1-yl)pyrimidin-2-yl)piperidin-4-yl)acetaldehyde (100 mg, 0.22 mmol) and sodium acetate (53 mg, 0.65 mmol) in DCM (1.4 mL) and MeOH (0.7 mL) was stirred at room temperature under nitrogen for 2 hours. Sodium triacetoxyhydroborate (137 mg, 0.65 mmol) was added and the resulting solution was stirred at 20° C. for 10 mm. The reaction was diluted with brine (200 mL) and extracted with DCM (3×50 mL). The combined organics were dried over MgSO₄, filtered and evaporated to dryness. The crude product was purified by preparative HPLC (Waters XSelect CSH C18 ODB column, 5p silica, 30 mm diameter, 100 mm length), using decreasingly polar mixtures of water (containing by volume 1% NH₄OH (28-30% in H₂O)) and MeCN (50-95% gradient) as eluents. Fractions containing the desired compound were evaporated cold, the resulting mixture was diluted with brine (30 mL) and extracted with DCM (3×20 mL). The combined organics were passed through a phase separation cartridge and concentrated under reduced pressure to afford the title compound (70 mg, 40%) as a white solid; ¹H NMR (400 MHz, DMSO, 30° C.) 1.09 (5H, d), 1.28 (4H, d), 1.37-1.5 (5H, m), 1.60 (1H, s), 1.74 (2H, d), 1.86-1.97 (1H, m), 2.21-2.44 (5H, m), 2.54-2.98 (7H, m), 3.08-3.21 (1H, m), 3.84 (3H, s), 4.11 (1H, d), 4.23 (1H, d), 4.61 (2H, d), 4.85-5.02 (2H, m), 6.48 (1H, s), 6.60 (1H, s), 6.92-7.1 (2H, m), 7.28 (1H, d), 7.44 (1H, d), 8.10 (2H, s), 10.71 (1H, s), 10.88 (1H, s), 6 protons obscured by DMSO and or water peaks; m/z: ES+ [M+H]+ 806.4.

Examples 13 to 41 (table below) were prepared using synthetic methods analogous to those described above.

| Ex No | Structure | Name | 1H NMR | LCMS |
|---|---|---|---|---|
| 13 | 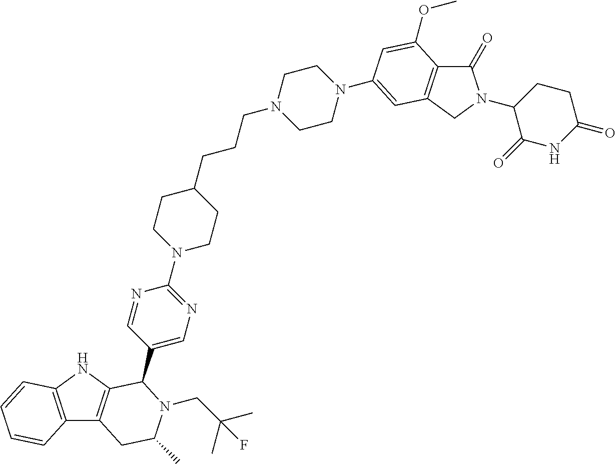 [SMILES: COc1cc(N2CCN(CCCC3CCN(c4ncc([C@@H]5c6[nH]c7ccccc7c6C[C@@H](C)N5CC(C)(C)F)cn4)CC3)CC2)cc2c1C(=O)N(C1CCC(=O)NC1=O)C2] | 3-(5-{4-[3-(1-{5- [(1R,3R)-2-(2- fluoro-2- methylpropyl)-3- methyl-2,3,4,9- tetrahydro-1H-beta- carbolin-1- yl]pyrimidin-2- yl}piperidin-4- yl)propyl]piperazin- 1-yl}-7-methoxy-1- oxo-1,3-dihydro- 2H-isoindol-2- yl)piperidine-2,6- dione | ¹H NMR (400 MHz, DMSO, 30° C.) 0.87-1.03 (2H, m), 1.08 (3H, d), 1.16-1.55 (12H, m), 1.68 (2H, d), 1.73-1.9 (2H, m), 1.9-2 (1H, m), 2.27-2.45 (5H, m), 2.56-2.96 (7H, m), 3.08-3.2 (1H, m), 3.23 (1H, d), 3.36 (1H, d), 3.55 (1H, s), 4.12- 4.37 (2H, m), 4.41 (1H, s), 4.60 (2H, d), 4.91 (1H, s), 5.03 (1H, dd), 6.67 (2H, d), 6.91-7.02 (1H, m), 7.02-7.13 (1H, m), 7.27 (1H, d), 7.45 (2H, dd), 8.08 (2H, s), 10.70 (1H, s), 10.91 (1H, s), 2 × aliphatic CH signals obscured by DMSO peak. | m/z: ES+ [M + H]+ = 820.5 |
| 14 | 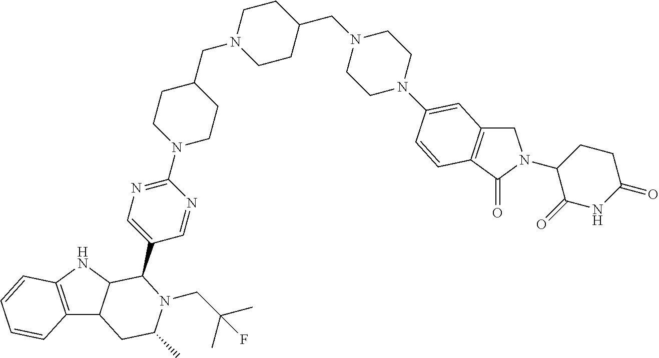 [SMILES: C[C@@H]1CC2c3ccccc3NC2[C@@H](c2cnc(N3CCC(CN4CCC(CN5CCN(c6ccc7c(c6)CN(C6CCC(=O)NC6=O)C7=O)CC5)CC4)CC3)nc2)N1CC(C)(C)F] | 3-{5-[4-({1-[(1-{5- [(1R,3R)-2-(2- fluoro-2- methylpropyl)-3- methyl-2,3,4,9- tetrahydro-1H-beta- carbolin-1- yl)pyrimidin-2- yl}piperidin-4- yl)methyl]piperidin- 4- yl}methyl)piperazin- 1-yl]-1-oxo-1,3- dihydro-2H- isoindol-2- yl}piperidine-2,6- dione | ¹H NMR (400 MHz, CDCl₃, 30 C.) 1.09 (3H, d), 1.11-1.34 (10H, m), 1.46 (3H, d), 1.67- 1.94 (7H, m), 2.11-2.26 (5H, m), 2.26-2.42 (1H, m), 2.46- 2.74 (7H, m), 2.74-2.98 (6H, m), 3.19-3.37 (4H, m), 4.25 (1H, d), 4.41 (1H, d), 4.71 (2H, d), 4.99 (1H, s), 5.19 (1H, dd), 6.87 (1H, s), 6.93-7.02 (1H, m), 7.07-7.19 (2H, m), 7.26- 7.31 (1H, m), 7.51 (1H, d), 7.64 (1H, s), 7.73 (1H, d), 7.77-7.98 (1H, m), 8.17 (2H, s) | m/z: ES+ [M + H]+ = 859.7 |
| 15 | 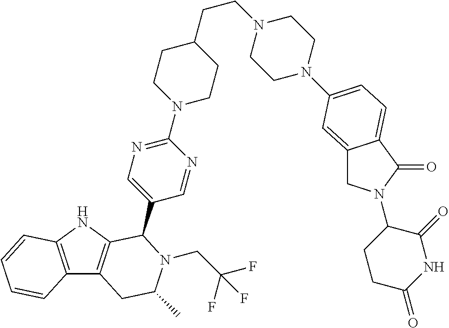 [SMILES: C[C@@H]1Cc2c([nH]c3ccccc23)[C@@H](c2cnc(N3CCC(CCN4CCN(c5ccc6c(c5)CN(C5CCC(=O)NC5=O)C6=O)CC4)CC3)nc2)N1CC(F)(F)F] | 3-(5-{4-[2-(1-{5- [(1R,3R)-3-methyl- 2-(2,2,2- trifluoroethyl)- 2,3,4,9-tetrahydro- 1H-beta-carbolin-1- yl]pyrimidin-2- yl}piperidin-4- yl)ethyl]piperazin- 1-yl}-1-oxo-1,3- dihydro-2H- isoindol-2- yl)piperidine-2,6- dione | ¹H NMR (300 MHz, DMSO, 25° C.) 1.01-1.16 (5H, m), 1.35- 1.49 (2H, m), 1.52-1.67 (1H, m), 1.74 (2H, d), 1.91-2.03 (1H, m), 2.26-2.42 (3H, m), 2.46-2.49 (2H, m), 2.52-2.64 (3H, m), 2.66-2.78 (1H, m), 2.78-2.95 (3H, m), 2.94-3.14 (1H, m), 3.18 (2H, d), 3.23- 3.32 (4H, m), 3.44-3.62 (1H, m), 4.20 (1H, d), 4.33 (1H, d), 4.62 (2H, d), 4.89 (1H, s), 4.99- 5.11 (1H, m), 6.94-7.13 (4H, m), 7.29 (1H, d), 7.45 (1H, d), 7.48-7.57 (1H, m), 8.06 (2H, s), 10.74 (1H, s), 10.95 (1H, s) | m/z (ES+), [M + H]+ = 784.4 |
| 16 | 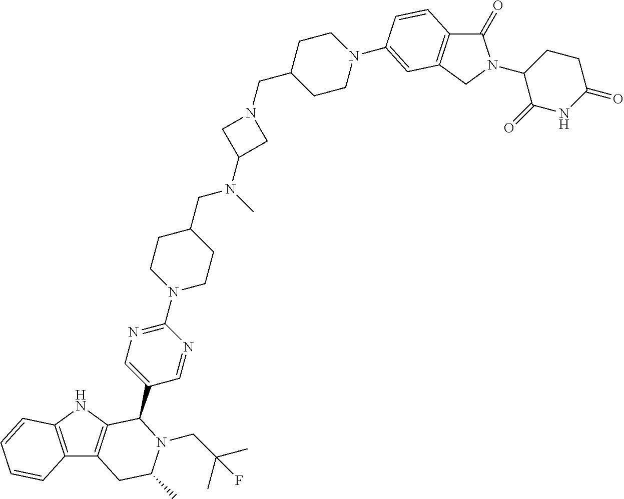 [SMILES: C[C@@H]1Cc2c([nH]c3ccccc23)[C@@H](c2cnc(N3CCC(CN(C)C4CN(CC5CCN(c6ccc7c(c6)CN(C6CCC(=O)NC6=O)C7=O)CC5)C4)CC3)nc2)N1CC(C)(C)F] | 3-(5-{4-[(3-{[(1-{5- [(1R,3R)-2-(2- fluoro-2- methylpropyl)-3- methyl-2,3,4,9- tetrahydro-1H-beta- carbolin-1- yl]pyrimidin-2- yl}piperidin-4- yl)methyl](methyl)a- mino}azetidin-1- yl)methyl]piperidin- 1-yl}-1-oxo-1,3- dihydro-2H- isoindol-2- yl)piperidine-2,6- dione | ¹H NMR (400 MHz, DMSO, 25° C.) 0.84-1.01 (3H, m), 1.08 (3H, d), 1.13-1.32 (6H, m), 1.34-1.51 (5H, m), 1.69-1.74 (5H, m), 1.97 (6H, d), 2.26- 2.49 (3H, m), 2.53-2.97 (12H, m), 3.13 (1H, s), 3.83 (2H, d), 4.18 (1H, d), 4.30 (1H, d), 4.60 (2H, d), 4.91 (1H, s), 4.99-5.08 (1H, m), 6.93-7.09 (4H, m), 7.27 (1H, d), 7.43 (1H, d), 7.48 (1H, d), 8.09 (2H, s), 10.73 (1H, s), 10.94 (1H, s) | m/z (ES+), [M + H]+ = 859.5 |
| 17 | 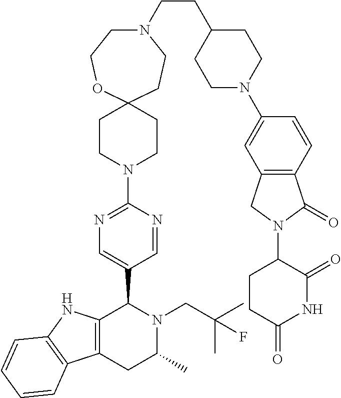 [SMILES: C[C@@H]1Cc2c([nH]c3ccccc23)[C@@H](c2cnc(N3CCC4(CCN(CCC5CCN(c6ccc7c(c6)CN(C6CCC(=O)NC6=O)C7=O)CC5)CCO4)CC3)nc2)N1CC(C)(C)F] | 3-(5-{4-[2-(3-{5- [(1R,3R)-2-(2- fluoro-2- methylpropyl)-3- methyl-2,3,4,9- tetrahydro-1H-beta- carbolin-1- yl]pyrimidin-2-yl}- 7-oxa-3,10- diazaspiro[5.6]dode- can-10- yl)ethyl]piperidin- 1-yl}-1-oxo-1,3- dihydro-2H- isoindol-2- yl)piperidine-2,6- dione | ¹H NMR (300 MHz, DMSO, 26° C.) 1.08 (3H, d), 1.13-1.57 (15H, m), 1.65-1.83 (6H, m), 1.91-2.01 (1H, m), 2.29-2.47 (4H, m), 2.57-2.69 (2H, m), 2.70-3.00 (4H, m), 3.07-3.28 (3H, m), 3.33 (3H, s), 3.57- 3.65 (2H, m), 3.85 (2H, d), 4.13- 4.39 (4H, m), 4.92 (1H, s), 4.98-5.10 (1H, m), 6.92-7.11 (4H, m), 7.28 (1H, d), 7.40- 7.54 (2H, m), 8.09 (2H, s), 10.73 (1H, s), 10.94 (1H, s) | m/z (ES+), [M + H]+ = 860.5 |
| 18 | 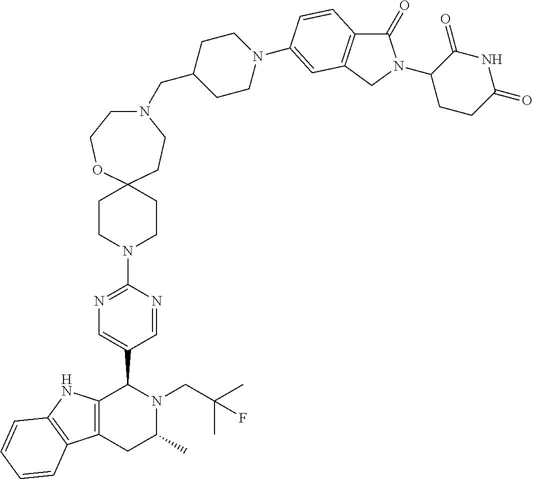 [SMILES: C[C@@H]1Cc2c([nH]c3ccccc23)[C@@H](c2cnc(N3CCC4(CCN(CC5CCN(c6ccc7c(c6)CN(C6CCC(=O)NC6=O)C7=O)CC5)CCO4)CC3)nc2)N1CC(C)(C)F] | 3-(5-{4-[(3-{5- [(1R,3R)-2-(2- fluoro-2- methylpropyl)-3- methyl-2,3,4,9- tetrahydro-1H-beta- carbolin-1- yl]pyrimidin-2-yl}- 7-oxa-3,10- diazaspiro[5.6]dode- can-10- yl)methyl]piperidin- 1-yl}-1-oxo-1,3- dihydro-2H- isoindol-2- yl)piperidine-2,6- dione | ¹H NMR (300 MHz, DMSO, 25° C.) 1.05-1.49 (13H, m), 1.67-1.82 (7H, m), 1.88-2.02 (2H, m), 2.18-2.42 (4H, m), 2.53-2.70 (4H, m), 2.71-3.05 (5H, m), 3.09-3.19 (1H, m), 3.18-3.32 (3H, m), 3.59-3.65 (2H, m), 3.81-3.91 (2H, m), 4.13-4.38 (4H, m), 4.92 (1H, s), 4.99-5.11 (1H, m), 6.92- 7.11 (4H, m), 7.28 (1H, d), 7.39- 7.56 (2H, m), 8.10 (2H, s), 10.74 (1H, s), 10.95 (1H, s) | m/z (ES+), [M + H]+ = 846.5 |
| 19 | 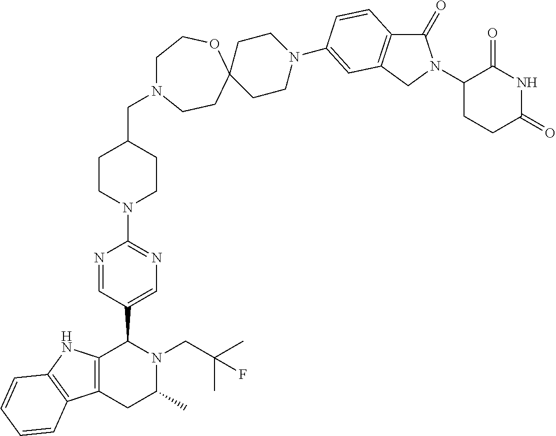 [SMILES: C[C@@H]1Cc2c([nH]c3ccccc23)[C@@H](c2cnc(N3CCC(CN4CCOC5(CC4)CCN(c4ccc6c(c4)CN(C4CCC(=O)NC4=O)C6=O)CC5)CC3)nc2)N1CC(C)(C)F] | 3-(5-{10-[(1-{5- [(1R,3R)-2-(2- fluoro-2- methylpropyl)-3- methyl-2,3,4,9- tetrahydro-1H-beta- carbolin-1- yl]pyrimidin-2- yl}piperidin-4- yl)methyl]-7-oxa- 3,10- diazaspiro[5.6]dode- can-3-yl}-1-oxo- 1,3-dihydro-2H- isoindol-2- yl)piperidine-2,6- dione | ¹H (400 MHz, DMSO, 30° C.) 0.93-1.03 (2H, m), 1.09 (3H, d), 1.28 (3H, d), 1.42 (3H, d), 1.46-1.54 (2H, m), 1.67-1.86 (7H, m), 1.91-2.01 (1H, m), 2.18-2.25 (2H, m), 2.36-2.42 (1H, m), 2.54-2.65 (3H, m), 2.7-2.97 (4H, m), 3.09-3.22 (3H, m), 3.5-3.58 (2H, m), 3.59-3.65 (2H, m), 4.19 (1H, d), 4.32 (1H, d), 4.61 (2H, d), 4.91 (1H, s), 5.03 (1H, dd), 6.94- 7.11 (4H, m), 7.27 (1H, d), 7.46 (2H, dd), 8.09 (2H, s), 10.70 (1H, s), 10.91 (1H, s)., 5 protons obscured by DMSO and/or water peaks | m/z (ES+), [M + H]+ = 846.5 |
| 20 | 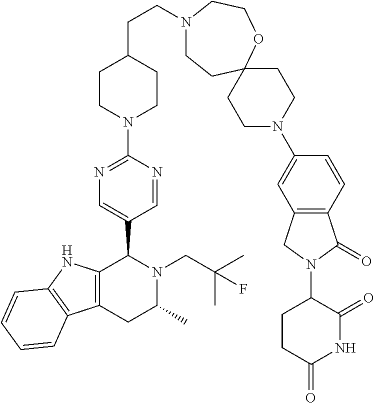 [SMILES: C[C@@H]1Cc2c([nH]c3ccccc23)[C@@H](c2cnc(N3CCC(CCN4CCOC5(CC4)CCN(c4ccc6c(c4)CN(C4CCC(=O)NC4=O)C6=O)CC5)CC3)nc2)N1CC(C)(C)F] | 3-(5-{10-[2-(1-{5- [(1R,3R)-2-(2- fluoro-2- methylpropyl)-3- methyl-2,3,5,9- tetrahydro-1H-beta- carbolin-1- yl]pyrimidin-2- yl}piperidin-4- yl)ethyl]-7-oxa- 3,10- diazaspiro[5.6]dode- can-3-yl}-1-oxo- 1,3-dihydro-2H- isoindol-2- yl)piperidine-2,6- dione | ¹H NMR (400 MHz, CDCl₃, 30° C.) 1.10 (3H, d), 1.13-1.25 (3H, m), 1.30 (3H, d), 1.46 (7H, d), 1.71-1.79 (2H, m), 1.81- 1.91 (4H, m), 2.15-2.23 (1H, m), 2.25-2.39 (1H, m), 2.43- 2.73 (10H, m), 2.75-2.94 (4H, m), 3.19-3.32 (3H, m), 3.47- 3.56 (2H, m), 3.66-3.73 (2H, m), 4.23 (1H, d), 4.39 (1H, d), 4.65-4.74 (2H, m), 4.99 (1H, s), 5.18 (1H, dd), 6.83-6.89 (1H, m), 6.94-7 (1H, m), 7.08- 7.13 (1H, m), 7.14-7.19 (1H, m), 7.28 (1H, s), 7.48-7.54 (1H, m), 7.66 (1H, s), 7.70 (1H, d), 7.88 (1H, s), 8.17 (2H, s) | m/z (ES+), [M + H]+ = 860.6 |
| 21 | 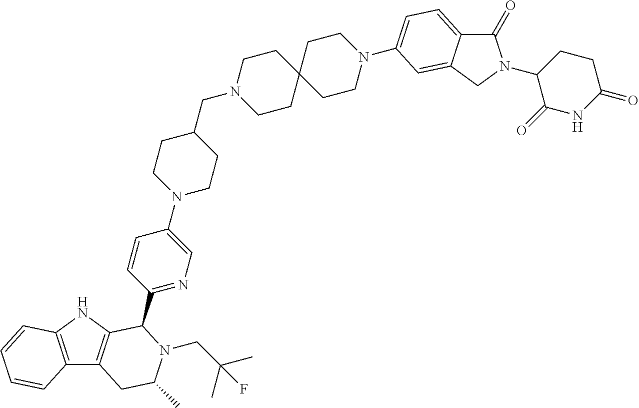 [SMILES: C[C@@H]1Cc2c([nH]c3ccccc23)[C@@H](c2ccc(N3CCC(CN4CCC5(CC4)CCN(c4ccc6c(c4)CN(C4CCC(=O)NC4=O)C6=O)CC5)CC3)cn2)N1CC(C)(C)F] | 3-(5-{9-[(1-{6- [(1S,3R)-2-(2- fluoro-2- methylpropyl)-3- methyl-2,3,4,9- tetrahydro-1H-beta- carbolin-1- yl]pyridin-3- yl}piperidin-4- yl)methyl]-3,9- diazaspiro[5.5]unde- can-3-yl}-1-oxo- 1,3-dihydro-2H- isoindol-2- yl)piperidine-2,6- dione | ¹H NMR (400 MHz, CDCl₃, 30° C.) 1.19-1.44 (9H, m), 1.51- 1.72 (11H, m), 1.86 (2H, d), 2.11-2.45 (8H, m), 2.54-2.99 (8H, m), 3.22-3.34 (4H, m), 3.32-3.48 (1H, m), 3.57-3.71 (2H, m), 4.23 (1H, d), 4.40 (1H, d), 5.03 (1H, s), 5.18 (1H,dd), 6.86 (1H, s), 6.97 (1H, dd), 7.05 (1H, td), 7.11 (1H, td), 7.19 (1H, dd), 7.31 (1H, d), 7.47 (1, d), 7.53 (1H, d), 7.71 (1H, d), 7.9 (1H, s), 8.22 (1H, d), 8.51 (1H, s) | m/z (ES+), [M + H]+ = 829.6 |
| 22 | 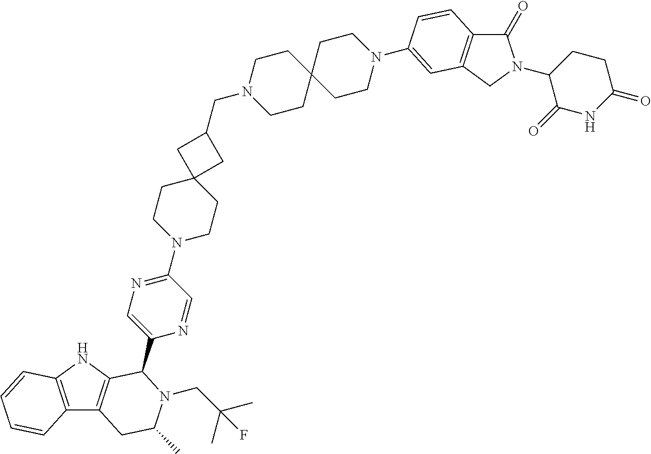 [SMILES: C[C@@H]1Cc2c([nH]c3ccccc23)[C@@H](c2cnc(N3CCC4(CC3)CC(CN3CCC5(CC3)CCN(c3ccc6c(c3)CN(C3CCC(=O)NC3=O)C6=O)CC5)C4)cn2)N1CC(C)(C)F] | 3-(5-{9-[(7-{5- [(1R,3R)-2-(2- fluoro-2- methylpropyl)-3- methyl-2,3,4,9- tetrahydro-1H-beta- carbolin-1- yl]pyrimidin-2-yl}- 7- azaspiro[3.5]nonan- 2-yl)methyl]-3,9- diazaspiro[5.5]unde- can-3-yl}-1-oxo- 1,3-dihydro-2H- isoindol-2- yl)piperidine-2,6- dione | ¹H NMR (300 MHz, DMSO, 23° C.) 1.08 (3H, d), 1.18-1.64 (22H, m), 1.88-2.00 (3H, m), 2.31-2.47 (7H, m), 2.55-2.70 (3H, m), 2.74-3.00 (2H, m), 3.13 (1H, s), 3.25-3.35 (4H, m), 3.55-3.73 (4H, m), 4.19 (1H, d), 4.32 (1H, d), 4.91 (1H, s), 4.98-5.11 (1H, m), 6.92- 7.11 (4H, m), 7.27 (1H, d), 7.39- 7.54 (2H, m), 8.08 (2H, s), 10.74 (1H, s), 10.95 (1H, s) | m/z (ES+), [M + H]+ = 870.6 |
| 23 | 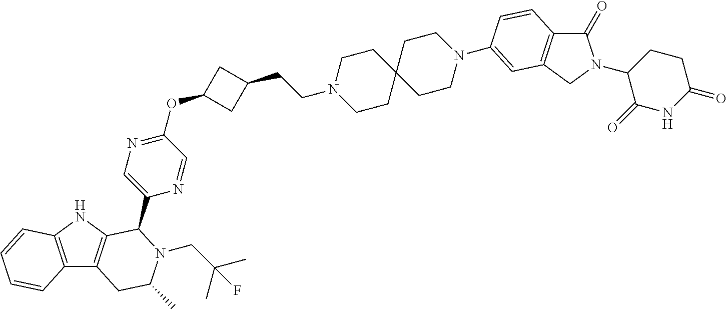 [SMILES: C[C@@H]1Cc2c([nH]c3ccccc23)[C@@H](c2cnc(O[C@H]3C[C@@H](CCN4CCC5(CC4)CCN(c4ccc6c(c4)CN(C4CCC(=O)NC4=O)C6=O)CC5)C3)cn2)N1CC(C)(C)F] | 3-[5-(9-{2-[(1S,3r)- 3-({5-[(1R,3R)-2- (2-fluoro-2- methylpropyl)-3- methyl-2,3,4,9- tetrahydro-1H-beta- carbolin-1- yl]pyrimidin-2- yl}oxy)cyclobutyl]eth- yl}-3,9- diazaspiro[5.5]unde- can-3-yl)-1-oxo- 1,3-dihydro-2H- isoindol-2- yl]piperidine-2,6- dione | ¹H NMR (300 MHz, DMSO, 26° C.) 1.09 (3H, d), 1.18-1.29 (3H, m), 1.31-1.36 (2H, m), 1.38-1.42 (1H, m), 1.44-1.54 (9H, m), 1.56-1.76 (4H, m), 1.82-2.01 (2H, m), 2.25-2.38 (3H, m), 2.36-2.47 (6H, m), 2.52-2.58 (6H, m), 2.59-3.11 (3H, m), 4.14-4.38 (2H, m), 4.87-5.13 (3H, m), 6.94-7.13 (4H, m), 7.30 (1H, d), 7.41- 7.54 (2H, m), 8.24 (1H, s), 8.34 (2H, s), 10.80 (1H, s), 10.94 (1H, s) | m/z (ES+), [M + H]+ = 831.5 |
| 24 | 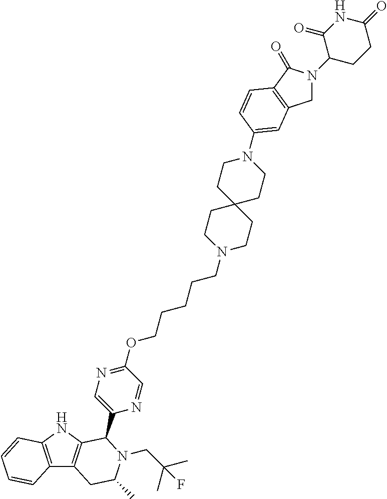 [SMILES: C[C@@H]1Cc2c([nH]c3ccccc23)[C@@H](c2cnc(OCCCCCN3CCC4(CC3)CCN(c3ccc5c(c3)CN(C3CCC(=O)NC3=O)C5=O)CC4)cn2)N1CC(C)(C)F] | 3-(5-{9-[5-({5- [(1R,3R)-2-(2- fluoro-2- methylpropyl)-3- methyl-2,3,4,9- tetrahydro-1H-beta- carbolin-1- yl]pyrimidin-2- yl}oxy)pentyl]-3,9- diazaspiro[5.5]unde- can-3-yl}-1-oxo- 1,3-dihydro-2H- isoindol-2- yl)piperidine-2,6- dione | ¹H NMR (400 MHz, CDCl₃, 30° C.) 1.11 (3H, d), 1.31 (3H, d), 1.42-1.74 (14H, m), 1.82 (2H, p), 2.18 (1H, dtd), 2.25-2.47 (7H, m), 2.5-2.74 (4H, m), 2.75-2.94 (2H, m), 3.1-3.23 (1H, m), 3.25-3.35 (4H, m), 4.24 (1H, d), 4.33 (2H, t), 4.39 (1H, d), 5.1-5.24 (2H, m), 6.85 (1H, d), 6.97 (1H, dd), 7.13 (1H, td), 7.19 (1H, td), 7.28-7.37 (2H, m), 7.54 (1H, d), 7.70 (1H, d), 7.84 (1H, s), 7.9-8.07 (1H, m), 8.38 (2H, s) | m/z (ES+), [M + H]+ = 819.5 |
| 25 | 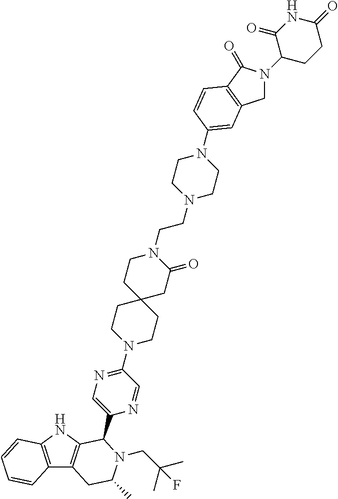 [SMILES: C[C@@H]1Cc2c([nH]c3ccccc23)[C@@H](c2cnc(N3CCC4(CCN(CCN5CCN(c6ccc7c(c6)CN(C6CCC(=O)NC6=O)C7=O)CC5)C(=O)C4)CC3)cn2)N1CC(C)(C)F] | 3-(5-{4-[2-(9-{5- [(1R,3R)-2-(2- fluoro-2- methylpropyl)-3- methyl-2,3,4,9- tetrahydro-1H-beta- carbolin-1- yl]pyrimidin-2-yl}- 2-oxo-3,9- diazaspiro[5.5]unde- can-3- yl)ethyl]piperazin- 1-yl}-1-oxo-1,3- dihydro-2H- isoindol-2- yl)piperidine-2,6- dione | ¹H NMR (400 MHz, DMSO, 30° C.) 1.08 (3H, d), 1.28 (3H, d), 1.34-1.51 (7H, m), 1.73 (2H, t), 1.87-2.01 (1H, m), 2.19 (2H, s), 2.3-2.42 (2H, m), 2.41- 2.49 (4H, m), 2.54-2.66 (5H, m), 2.68-2.81 (1H, m), 2.83- 2.97 (1H, m), 3.14 (1H, s), 3.21- 3.28 (4H, m), 3.36 (2H, t), 3.45 (2H, t), 3.65-3.82 (4H, m), 4.21 (1H, d), 4.33 (1H, d), 4.91 (1H, s), 5.04 (1H, dd), 6.95- 7.02 (1H, m), 7.02-7.09 (3H, m), 7.27 (1H, d), 7.43 (1H, d), 7.52 (1H, d), 8.10 (2H, s), 10.70 (1H, s), 10.92 (1H, s) | m/z (ES+), [M + H]+ = 859.6 |
| 26 | 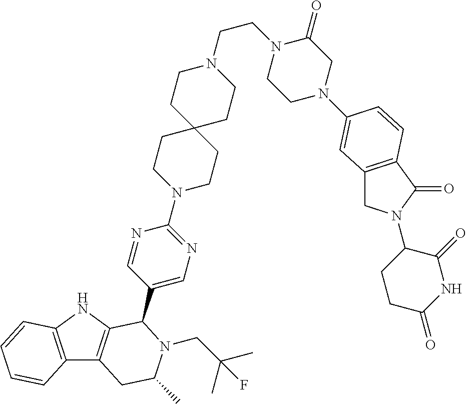 [SMILES: C[C@@H]1Cc2c([nH]c3ccccc23)[C@@H](c2cnc(N3CCC4(CCN(CCN5CCN(c6ccc7c(c6)CN(C6CCC(=O)NC6=O)C7=O)CC5=O)CC4)CC3)nc2)N1CC(C)(C)F] | 3-(5-{4-[2-(9-{5- [(1R,3R)-2-(2- fluoro-2- methylpropyl)-3- methyl-2,3,4,9- tetrahydro-1H-beta- carbolin-1- yl]pyrimidin-2-yl}- 3,9- diazaspiro[5.5]unde- can-3-yl)ethyl]-3- oxopiperazin-1-yl}- 1-oxo-1,3-dihydro- 2H-isoindol-2- yl)piperidine-2,6- dione | ¹H NMR (400 MHz, DMSO, 30° C.) 1.08 (3H, d), 1.28 (4H, d), 1.51-1.32 (m, 11H), 1.87- 2.02 (1H, m), 2.28-2.41 (5H, m), 2.46 (3H, t), 2.54-2.65 (2H, m), 2.75 (1H, dd), 2.82- 2.94 (1H, m), 3.15 (1H, s), 3.47 (2H, t), 3.51-3.57 (2H, m), 3.61 (2H, d), 3.65-3.73 (4H, m), 3.92 (2H, s), 4.23 (1H, d), 4.33 (1H, d), 4.91 (1H, s), 5.02 (1H, dd), 6.92-7.01 (1H, m), 7- 7.08 (3H, m), 7.28 (1H, d), 7.43 (1H, d), 7.55 (1H, d), 8.09 (2H, s), 10.71 (1H, s), 10.90 (1H, s).) | m/z (ES+), [M + H]+ = 859.7 |
| 27 | 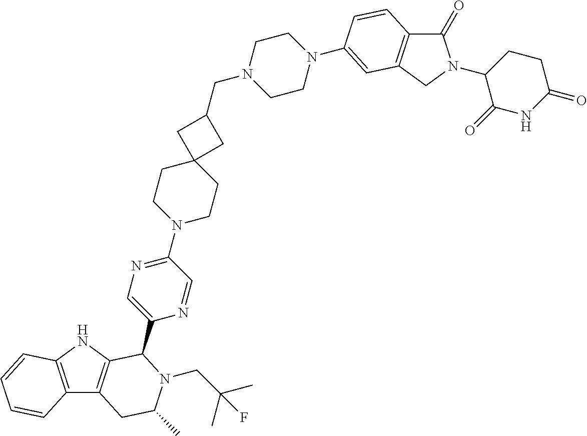 [SMILES: C[C@@H]1Cc2c([nH]c3ccccc23)[C@@H](c2cnc(N3CCC4(CC3)CC(CN3CCN(c5ccc6c(c5)CN(C5CCC(=O)NC5=O)C6=O)CC3)C4)cn2)N1CC(C)(C)F] | 3-(5-{4-[(7-{5- [(1R,3R)-2-(2- fluoro-2- methylpropyl)-3- methyl-2,3,4,9- tetrahydro-1H-beta- carbolin-1- yl]pyrimidin-2-yl}- 7- azaspiro[3.5]nonan- 2- yl)methyl]piperazin- 1-yl}-1-oxo-1,3- dihydro-2H- isoindol-2- yl)piperidine-2,6- dione | ¹H NMR (400 MHz, CDCl₃, 30° C.) 1.10 (3H, d), 1.40-1.25 (6H, m), 1.58-1.74 (8H, m), 2.17-2.27 (3H, m), 2.34 (1H, dd), 2.47-2.73 (4H, m), 2.74- 2.98 (4H, m), 3-3.13 (3H, m), 3.18-3.3 (1H, m), 3.62-3.88 (8H, m), 4.24-4.46 (2H, m), 5.00 (1H, s), 5.18 (1H, dd), 6.88- 6.92 (1H, m), 6.97-7.02 (1H, m), 7.08-7.19 (2H, m), 7.27- 7.3 (1H, m), 7.49-7.54 (1H, m), 7.64 (1H, s), 7.77 (1H, d), 7.86 (1H, s), 8.18 (2H, s) | m/z (ES+), [M + H]+ = 803.0 |
| 28 | 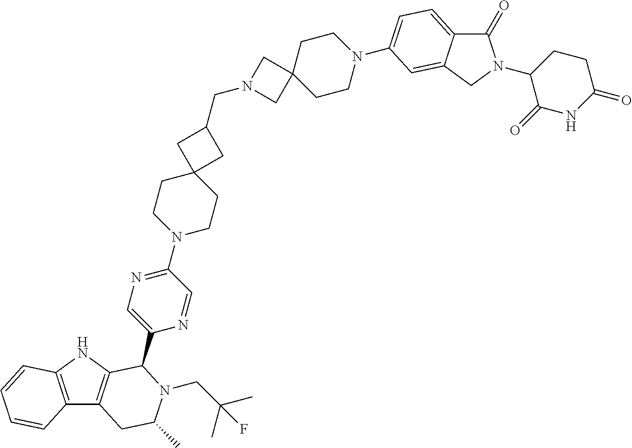 [SMILES: C[C@@H]1Cc2c([nH]c3ccccc23)[C@@H](c2cnc(N3CCC4(CC3)CC(CN3CC5(CCN(c6ccc7c(c6)CN(C6CCC(=O)NC6=O)C7=O)CC5)C3)C4)cn2)N1CC(C)(C)F] | 3-(5-{2-[(7-{5- [(1R,3R)-2-(2- fluoro-2- methylpropyl)-3- methyl-2,3,4,9- tetrahydro-1H-beta- carbolin-1- yl]pyrimidin-2-yl}- 7- azaspiro[3.5]nonan- 2-yl)methyl]-2,7- diazaspiro[3.5]nonan- 7-yl}-1-oxo-1,3- dihydro-2H- isoindol-2- yl)piperidine-2,6- dione | ¹H NMR (300 MHz, DMSO, 23° C.) 1.08 (3H, d), 1.24-1.48 (10H, m), 1.48-1.60 (2H, m), 1.71-1.77 (3H, m), 1.82-2.00 (3H, m), 2.19-2.40 (2H, m), 2.41-2.49 (4H, m), 2.56-2.69 (2H, m), 2.71-3.03 (6H, m), 3.09-3.16 (1H, m), 3.24-3.30 (4H, m), 3.54-3.74 (4H, m), 4.11-4.40 (2H, m), 4.91 (1H, s), 4.98-5.10 (1H, m), 6.92- 7.11 (4H, m), 7.23-7.32 (1H, m), 7.39-7.54 (2H, m), 8.08 (2H, s), 10.74 (1H, s), 10.96 (1H, s) | m/z (ES+), [M + H]+ = 842.5 |
| 29 | 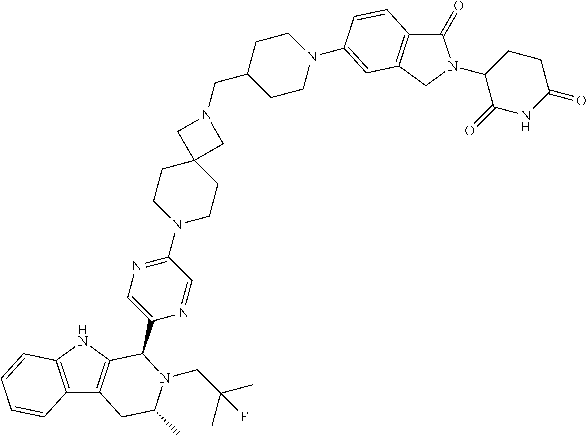 [SMILES: C[C@@H]1Cc2c([nH]c3ccccc23)[C@@H](c2cnc(N3CCC4(CC3)CN(CC3CCN(c5ccc6c(c5)CN(C5CCC(=O)NC5=O)C6=O)CC3)C4)cn2)N1CC(C)(C)F] | 3-(5-{4-[(7-{5- [(1R,3R)-2-(2- fluoro-2- methylpropyl)-3- methyl-2,3,4,9- tetrahydro-1H-beta- carbolin-1- yl]pyrimidin-2-yl}- 2,7- diazaspiro[3.5]nonan- 2- yl)methyl]piperidin- 1-yl}-1-oxo-1,3- dihydro-2H- isoindol-2- yl)piperidine-2,6- dione | ¹H NMR (400 MHz, CDCl₃, 30° C.) 1.08 (3H, d), 1.23-1.52 (8H, m), 1.65-1.85 (2H, m), 1.97 (2H, d), 2.04-2.22 (4H, m), 2.24-2.39 (1H, m), 2.46-2.7 (4H, m), 2.75-2.92 (4H, m), 3.02 (2H, d), 3.10-3.25 (1H, m), 3.45-3.59 (2H, m), 3.63-3.85 (6H, m), 4.17-4.4 (4H, m), 5.01 (1H, s), 5.13 (1H, dd), 6.82 (1H, s), 6.90 (1H, d), 7.10-7.25 (2H, m), 7.30 (1H, d), 7.50 (1H, d), 7.65 (1H, dd), 8.17 (2H, s), 8.22-8.28 (1H, m), 8.28-8.34 (1H, m), 12.72 (1H, s); formate salt | m/z (ES+), [M + H]+ = 802.5 |
| 30 | 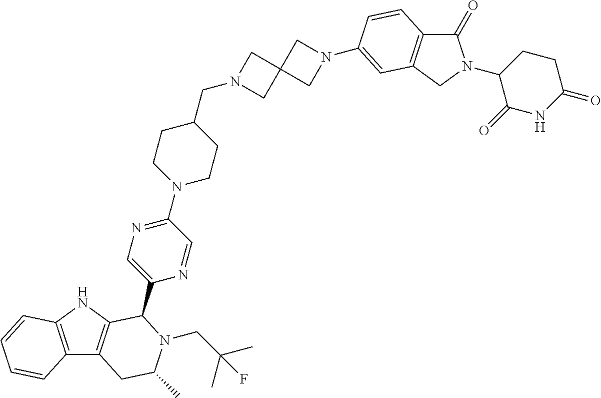 [SMILES: C[C@@H]1Cc2c([nH]c3ccccc23)[C@@H](c2cnc(N3CCC(CN4CC5(C4)CN(c4ccc6c(c4)CN(C4CCC(=O)NC4=O)C6=O)C5)CC3)cn2)N1CC(C)(C)F] | 3-(5-{6-[(1-{5- [(1R,3R)-2-(2- fluoro-2- methylpropyl)-3- methyl-2,3,4,9- tetrahydro-1H-beta- carbolin-1- yl]pyrimidin-2- yl}piperidin-4- yl)methyl]-2,6- diazaspiro[3.3]hept- an-2-yl}-1-oxo-1,3- dihydro-2H- isoindol-2- yl)piperidine-2,6- dione | ¹H NMR (300 MHz, CDCl₃, 26° C.) 1.08-1.39 (6H, m), 1.49 (3H, d), 1.64 (2H, d), 1.78 (2H, d), 2.15-2.45 (5H, m), 2.49- 2.70 (4H, m), 2.73-2.91 (4H, m), 3.26 (1H, s), 3.48 (4H, s), 4.03 (4H, s), 4.23 (1H, d), 4.38 (1H, d), 4.72 (2H, d), 5.00 (1H, s), 5.13-5.25 (1H, m), 6.38 (1H, s), 6.40-6.50 (1H, m), 7.07-7.23 (2H, m), 7.31 (1H, s), 7.53 (1H, d), 7.69 (1H, d), 8.01 (1H, s), 8.18 (2H, s), 8.43 (1H, s) | m/z (ES+), [M + H]+ = 774.4 |
| 31 | 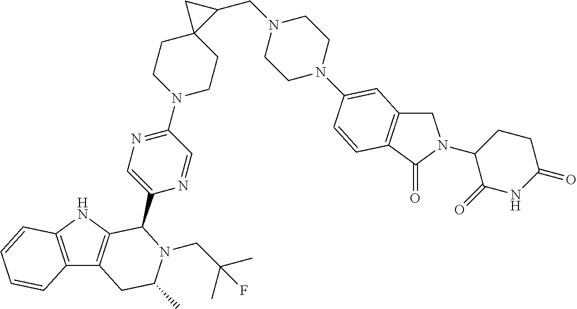 [SMILES: C[C@@H]1Cc2c([nH]c3ccccc23)[C@@H](c2cnc(N3CCC4(CC3)CC4CN3CCN(c4ccc5c(c4)CN(C4CCC(=O)NC4=O)C5=O)CC3)cn2)N1CC(C)(C)F] | 3-(5-{4-[(6-{5- [(1R,3R)-2-(2- fluoro-2- methylpropyl)-3- methyl-2,3,4,9- tetrahydro-1H-beta- carbolin-1- yl]pyrimidin-2-yl}- 6- azaspiro[2.5]octan- 1- yl)methyl]piperazin- 1-yl}-1-oxo-1,3- dihydro-2H- isoindol-2- yl)piperidine-2,6- dione | ¹H NMR (400 MHz, CDCl₃, 30° C.) 0.50-0.64 (1H, m), 0.78- 0.92 (1H, m), 1.08 (3H, d), 1.19-1.52 (10H, m), 1.59- 1.74 (2H, m), 2.06-2.35 (2H, m), 2.46-2.71 (4H, m), 2.75- 3.00 (3H, m), 3.00-3.50 (6H, m), 3.52-3.60 (5H, m), 4.14-4.4 (4H, m), 5.00 (1H, s), 5.04- 5.17 (1H, m), 6.73-6.96 (2H, m), 7.03-7.18 (2H, m), 7.27- 7.35 (1H, m), 7.50 (1H, d), 7.57- 7.71 (1H, m), 8.17 (2H, s), 8.27-8.44 (1H, m), 8.60-8.78 (1H, m) | m/z (ES+), [M + H]+ = 789.0 |
| 32 | 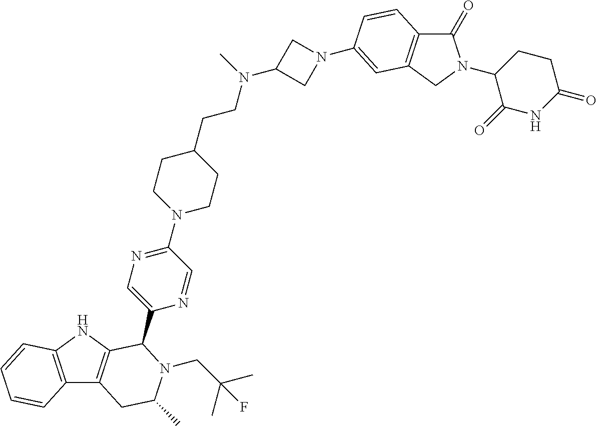 [SMILES: C[C@@H]1Cc2c([nH]c3ccccc23)[C@@H](c2cnc(N3CCC(CCN(C)C4CN(c5ccc6c(c5)CN(C5CCC(=O)NC5=O)C6=O)C4)CC3)cn2)N1CC(C)(C)F] | 3-[5-(3-{[2-(1-{5- [(1R,3R)-2-(2- fluoro-2- methylpropyl)-3- methyl-2,3,4,9- tetrahydro-1H-beta- carbolin-1- yl]pyrimidin-2- yl}piperidin-4- yl)ethyl](methyl)ami- no}azetidin-1-yl)- 1-oxo-1,3-dihydro- 2H-isoindol-2- yl]piperidine-2,6- dione | ¹H NMR (400 MHz, CDCl₃, 30° C.) 1.09 (3H, d), 1.14-1.35 (6H, m), 1.39-1.51 (5H, m), 1.75 (2H, d), 2.14-2.23 (4H, m), 2.23-2.42 (3H, m), 2.47- 2.74 (4H, m), 2.75-2.98 (4H, m), 3.18-3.34 (1H, m), 3.42 (1H, p), 3.75 (2H, t), 4.03 (2H, t), 4.22 (1H, d), 4.38 (1H, d), 4.70 (2H, d), 4.99 (1H, s), 5.17 (1H, dd), 6.38 (1H, s), 6.46 (1H, dd), 7.11 (1H, td), 7.16 (1H, td), 7.25-7.32 (1H, m), 7.48-7.54 (1H, m), 7.61-7.72 (2H, m), 7.79-8.00 (1H, m), 8.17 (2H, s) | m/z (ES+), [M + H]+ = 776.5 |
| 33 | 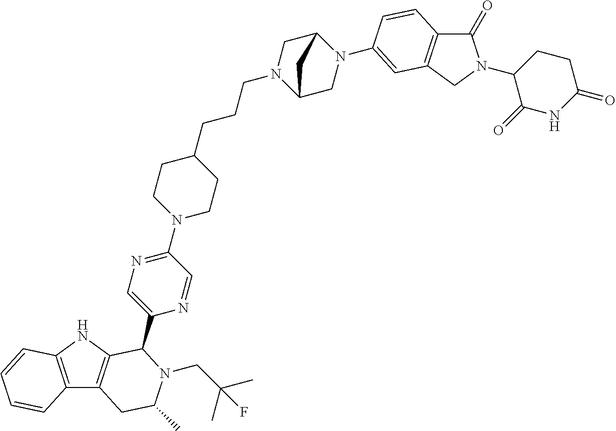 [SMILES: C[C@@H]1Cc2c([nH]c3ccccc23)[C@@H](c2cnc(N3CCC(CCCN4C[C@H]5C[C@@H]4CN5c4ccc5c(c4)CN(C4CCC(=O)NC4=O)C5=O)CC3)cn2)N1CC(C)(C)F] | 3-(5-{(1R,4R)-5-[3- (1-{5-[(1R,3R)-2- (2-fluoro-2- methylpropyl)-3- methyl-2,3,4,9- tetrahydro-1H-beta- carbolin-1- yl]pyrimidin-2- yl}piperidin-4- yl)propyl]-2,5- diazabicyclo[2.2.1] heptan-2-yl}-1-oxo- 1,3-dihydro-2H- isoindol-2- yl)piperidine-2,6- dione | ¹H NMR (400 MHz, DMSO, 30° C.) 0.87-1.03 (2H, m), 1.08 (3H, d), 1.16-1.55 (12H, m), 1.68 (2H, d), 1.73-1.9 (2H, m), 1.9-2.0 (1H, m), 2.27-2.45 (5H, m), 2.56-2.96 (7H, m), 3.08-3.2 (1H, m), 3.23 (1H, d), 3.36 (1H, d), 3.55 (1H, s), 4.12- 4.37 (2H, m), 4.41 (1H, s), 4.60 (2H, d), 4.91 (1H, s), 5.03 (1H, dd), 6.67 (2H, d), 6.91-7.02 (1H, m), 7.02-7.13 (1H, m), 7.27 (1H, d), 7.45 (2H, dd), 8.08 (2H, s), 10.70 (1H, s), 10.91 (1H, s) | m/z (ES+), [M + H]+ = 802.4 |
| 34 | 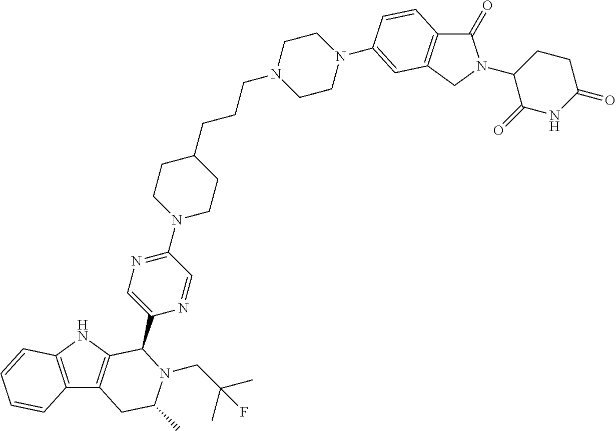 | 3-(5-{4-[3-(1-{5- [(1R,3R)-3-methyl- 2-(2,2,2- trifluoroethyl)- 2,3,4,9-tetrahydro- 1H-beta-carbolin-1- yl]pyrimidin-2- yl}piperidin-4- yl)propyl]piperazin- 1-yl}-1-oxo-1,3- dihydro-2H- isoindol-2- yl)piperidine-2,6- dione | ¹H (400 MHz, DMSO, 30° C.) 0.87-1.09 (2H, m), 1.13 (3H, d), 1.19-1.3 (3H, m), 1.41- 1.62 (3H, m), 1.66-1.8 (2H, m), 1.89-2.04 (1H, m), 2.21- 2.45 (4H, m), 2.55-2.79 (4H, m), 2.79-2.98 (3H, m), 2.98- 3.14 (1H, m), 3.14-3.22 (1H, m), 3.28 (3H, s), 3.44-3.65 (1H, m), 4.21 (1H, d), 4.33 (1H, d), 4.64 (2H, d), 4.88 (1H, s), 5.04 (1H, dd), 6.93-7.03 (1H, m), 7.03-7.12 (3H, m), 7.29 (1H, d), 7.45 (1H, d), 7.52 (1H, d), 8.06 (2H, s), 10.71 (1H, s), 10.92 (1H, s), 2 × aliphatic CH signals obscured by DMSO or water peaks | m/z (ES+), [M + H]+ = 798.5 |
| 35 | 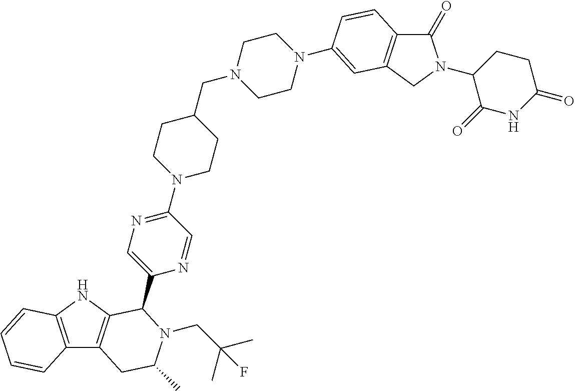 | 3-(5-{4-[(1-{5- [(1R,3R)-3-methyl- 2-(2,2,2- trifluoroethyl)- 2,3,4,9-tetrahydro- 1H-beta-carbolin-1- yl]pyrimidin-2- yl}piperidin-4- yl)methyl]piperazin- 1-yl}-1-oxo-1,3- dihydro-2H- isoindol-2- yl)piperidine-2,6- dione | ¹H NMR (400 MHz, DMSO, 30° C.) 0.93-1.08 (2H, m), 1.13 (3H, d), 1.23 (1H, d), 1.72- 2.02 (4H, m), 2.20 (2H, d), 2.27- 2.44 (2H, m), 2.52-2.77 ( 5H, m), 2.8-2.97 (3H, m), 2.97- 3.24 (2H, m), 3.45-3.66 (1H, m), 4.21 (1H, d), 4.33 (1H, d), 4.64 (2H, d), 4.89 (1H, s), 5.05 (1H, dd), 6.88-7.03 (1H, m), 7.03-7.16 (3H, m), 7.29 (1H, d), 7.49 (2H, dd), 8.07 (2H, s), 10.71 (1H, s), 10.92 (1H, s), 4 × proton obscured by DMSO and/or water peaks | m/z (ES+), [M + H]+ = 770.4 |
| 36 | 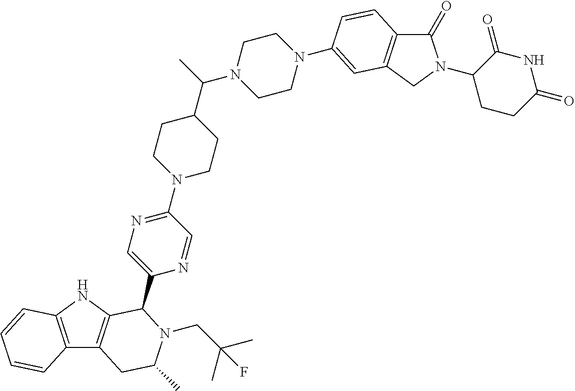 [SMILES: CC(C1CCN(c2cnc([C@@H]3c4[nH]c5ccccc5c4C[C@@H](C)N3CC(C)(C)F)cn2)CC1)N1CCN(c2ccc3c(c2)CN(C2CCC(=O)NC2=O)C3=O)CC1] | 3-(5-{4-[1-(1-{5- [(1R,3R)-2-(2- fluoro-2- methylpropyl)-3- methyl-2,3,4,9- tetrahydro-1H-beta- carbolin-1- yl]pyrimidin-2- yl}piperidin-4- yl)ethyl]piperazin- 1-yl}-1-oxo-1,3- dihydro-2H- isoindol-2- yl)piperidine-2,6- dione | ¹H NMR (400 MHz, DMSO, 30° C.) 0.91 (3H, d), 0.97-1.06 (2H, m), 1.08 (3H, d), 1.22- 1.32 (4H, m), 1.41 (3H, d), 1.57- 1.77 (2H, m), 1.91-2.01 (1H, m), 2.07 (1H, s), 2.19-2.48 (6H, m), 2.52-2.96 (8H, m), 3.08-3.28 (4H, m), 4.20 (1H, d), 4.32 (1H, d), 4.65 (2H, s), 4.91 (1H, s), 5.04 (1H, dd), 6.93- 7 (1H, m), 7.01-7.09 (3H, m), 7.27 (1H, d), 7.43 (1H, d), 7.51 (1H, d), 8.09 (2H, s), 10.70 (1H, s), 10.91 (1H, s) | m/z (ES+), [M + H]+ = 776.5 |
| 37 | 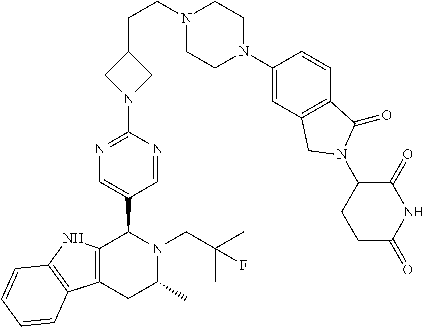 [SMILES: C[C@@H]1Cc2c([nH]c3ccccc23)[C@@H](c2cnc(N3CC(CCN4CCN(c5ccc6c(c5)CN(C5CCC(=O)NC5=O)C6=O)CC4)C3)nc2)N1CC(C)(C)F] | 3-(5-{4-[2-(1-{5- [(1R,3R)-2-(2- fluoro-2- methylpropyl)-3- methyl-2,3,4,9- tetrahydro-1H-beta- carbolin-1- yl]pyrimidin-2- yl}azetidin-3- yl)ethyl]piperazin- 1-yl}-1-oxo-1,3- dihydro-2H- isoindol-2- yl)piperidine-2,6- dione | ¹H NMR (400 MHz, CDCl3, 30° C.) 1.09 (3H, d), 1.26-1.32 (4H, m), 1.46 (3H, d), 1.89 (2H, q), 2.20 (1H, ddq), 2.25-2.38 (1H, m), 2.38-2.45 (2H, m), 2.53 (1H, dd), 2.57-2.63 (4H, m), 2.65 (1H, s), 2.69 (1H, d), 2.78 (1H, dd), 2.84 (1H, dd), 2.90 (1H, ddd), 3.24 (1H, s), 3.29-3.34 (4H, m), 3.75-3.81 (2H, m), 4.2-4.28 (3H, m), 4.41 (1H, d), 5.00 (1H, s), 5.19 (1H, dd), 6.87 (1H, s), 6.98 (1H, dd), 7.11 (1H, td), 7.16 (1H, td), 7.28 (1H, d), 7.42-7.55 (1H, m), 7.70 (1H, s), 7.73 (1H, d), 7.86 (1H, s), 8.20 (2H, s). | m/z (ES+), [M + H]+ = 748.4 |
| 38 | 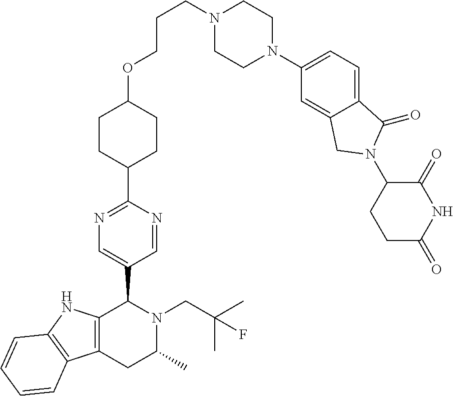 [SMILES: C[C@@H]1Cc2c([nH]c3ccccc23)[C@@H](c2cnc(C3CCC(OCCCN4CCN(c5ccc6c(c5)CN(C5CCC(=O)NC5=O)C6=O)CC4)CC3)nc2)N1CC(C)(C)F] | 3-[5-(4-{3-[(1-{5- [(1R,3R)-2-(2- fluoro-2- methylpropyl)-3- methyl-2,3,4,9- tetrahydro-1H-beta- carbolin-1- yl]pyrimidin-2- yl}piperidin-4- yl)oxy]propyl}piper- azin-1-yl)-1-oxo- 1,3-dihydro-2H- isoindol-2- yl]piperidine-2,6- dione | ¹H NMR (400 MHz, DMSO, 30° C.) 1.09 (3H, d), 1.28 (3H, d), 1.41 (5H, m), 1.63-1.76 (2H, m), 1.83 (2H, m), 1.91-2.03 (1H, m), 2.28-2.44 (4H, m), 2.55 (4H, m), 2.55-2.59 (1H, m), 2.59-2.71 (3H, m), 2.72- 2.83 (1H, m), 2.84-2.97 (1H, m), 3.15 (1H, m), 3.27 (3H, m), 3.32-3.41 (2H, m), 3.51 (3H, m), 4.1-4.37 (4H, m), 4.92 (1H, s), 5.04 (1H, dd), 6.95- 7.02 (1H, m), 7.02-7.11 (3H, m), 7.28 (1H, d), 7.44 (1H, d), 7.52 (1H, d), 8.11 (2H, s), 10.71 (1H, s), 10.92 (1H, s) | m/z (ES+), [M + H]+ = 806.4 |
| 39 | 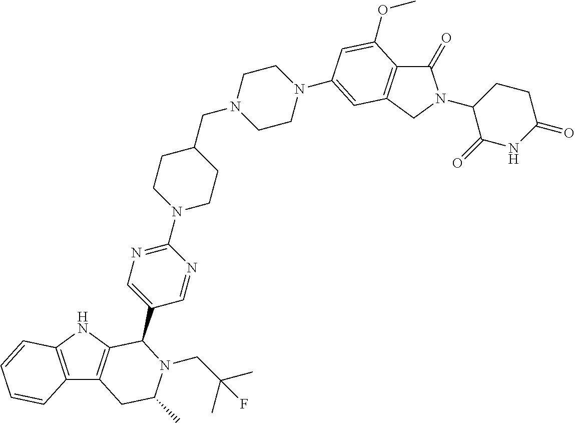 [SMILES: COc1cc(N2CCN(CC3CCN(c4ncc([C@@H]5c6[nH]c7ccccc7c6C[C@@H](C)N5CC(C)(C)F)cn4)CC3)CC2)cc2c1C(=O)N(C1CCC(=O)NC1=O)C2] | 3-(5-{4-[(1-{5- [(1R,3R)-2-(2- fluoro-2- methylpropyl)-3- methyl-2,3,4,9- tetrahydro-1H-beta- carbolin-1- yl]pyrimidin-2- yl}piperidin-4- yl)methyl]piperazin- 1-yl}-7-methoxy- 1-oxo-1,3-dihydro- 2H-isoindol-2- yl)piperidine-2,6- dione | ¹H NMR (400 MHz, DMSO, 26° C.) 0.82-1.14 (6H, m), 1.20- 1.35 (4H, m), 1.41 (3H, d), 1.74-1.96 (4H, m), 2.18 (2H, d), 2.24-2.39 (1H, m), 2.49- 2.51 (4H, m), 2.53-2.72 (2H, m), 2.67-2.96 (4H, m), 3.14 (1H, s), 3.30-3.34 (4H, m), 3.83 (3H, s), 4.10 (1H, d), 4.23 (1H, d), 4.63 (2H, d), 4.86-5.11 (2H, m), 6.45-6.50 (1H, m), 6.60 (1H, s), 6.93-7.01 (1H, m), 7.01-7.09 (1H, m), 7.27 (1H, d), 7.43 (1H, d), 8.10 (2H, s), 10.72 (1H, s), 10.89 (1H, s) | m/z (ES+), [M + H]+ = 792.5 |
| 40 | 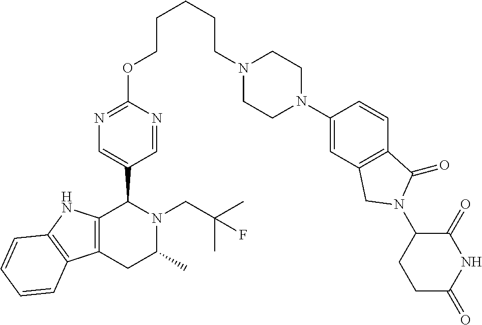 [SMILES: C[C@@H]1Cc2c([nH]c3ccccc23)[C@@H](c2cnc(OCCCCCN3CCN(c4ccc5c(c4)CN(C4CCC(=O)NC4=O)C5=O)CC3)nc2)N1CC(C)(C)F] | 3-(5-{4-[5-({5- [(1R,3R)-2-(2- fluoro-2- methylpropyl)-3- methyl-2,3,4,9- tetrahydro-1H-beta- carbolin-1- yl]pyrimidin-2- yl}oxy)pentyl]piper- azin-1-yl}-1-oxo- 1,3-dihydro-2H- isoindol-2- yl)piperidine-2,6- dione | ¹H NMR (400 MHz, CDCl₃, 30° C.) 1.10 (3H, d), 1.2-1.37 (9H, m), 1.41-1.57 (2H, m), 1.75-1.97 (3H, m), 2.11-2.38 (2H, m), 2.51-2.73 (4H, m), 2.77-3.02 (4H, m), 3.1-3.35 (4H, m), 3.58-3.72 (3H, m), 4.16-4.43 (4H, m), 5.07-5.2 (2H, m), 6.83-6.98 (2H, m), 7.04-7.18 (2H, m), 7.33 (1H, d), 7.52 (1H, d), 7.67 (1H, d), 8.38 (2H, s), 8.48-8.77 (2H, m) | m/z (ES+), [M + H]+ = 751.4 |
| 41 | 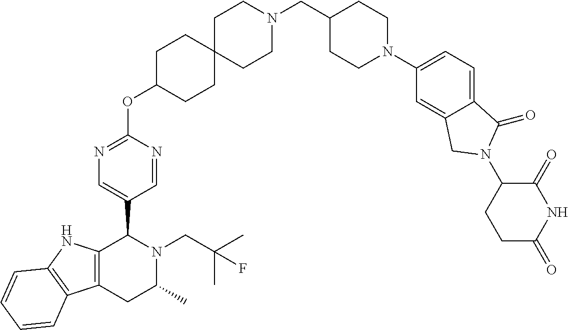 [SMILES: C[C@@H]1Cc2c([nH]c3ccccc23)[C@@H](c2cnc(OC3CCC4(CC3)CCN(CC3CCN(c5ccc6c(c5)CN(C5CCC(=O)NC5=O)C6=O)CC3)CC4)nc2)N1CC(C)(C)F] | 3-[5-(4-{[9-({5- [(1R,3R)-2-(2- fluoro-2- methylpropyl)-3- methyl-2,3,4,9- tetrahydro-1H-beta- carbolin-1- yl]pyrimidin-2- yl}oxy)-3- azaspiro[5.5]undecan- 3- yl]methyl}piperidin- 1-yl)-1-oxo-1,3- dihydro-2H- isoindol-2- yl]piperidine-2,6- dione | ¹H NMR (400 MHz, CDCl₃, 30° C.) 1.11 (3H, d), 1.22-1.38 (5H, m), 1.41-1.8 (14H, m), 1.82-1.94 (4H, m), 2.15-2.23 (3H, m), 2.24-2.41 (5H, m), 2.49-2.73 (4H, m), 2.76-2.96 (4H, m), 3.1-3.24 (1H, m), 3.82 (2H, d), 4.23 (1H, d), 4.39 (1H, d), 4.88-5.05 (1H, m), 5.1- 5.23 (2H, m), 6.86 (1H, d), 6.98 (1H, dd), 7.13 (1H, td), 7.19 (1H, td), 7.32 (1H, d), 7.53 (1H, d), 7.70 (1H, d), 7.78 (1H, s), 7.91 (1H, s), 8.36 (2H, s) | m/z (ES+), [M + H]+ = 845.6 |

The above description of illustrative embodiments is intended only to acquaint others skilled in the art with the Applicant's specification, its principles, and its practical application so that others skilled in the art may readily adapt and apply the specification in its numerous forms, as they may be best suited to the requirements of a particular use. This description and its specific examples, while indicating embodiments of this specification, are intended for purposes of illustration only. This specification, therefore, is not limited to the illustrative embodiments described in this specification, and may be variously modified. In addition, it is to be appreciated that various features of the specification that are, for clarity reasons, described in the context of separate embodiments, also may be combined to form a single embodiment. Conversely, various features of the specification that are, for brevity reasons, described in the context of a single embodiment, also may be combined to form sub-combinations thereof.

1. A compound of Formula (I): 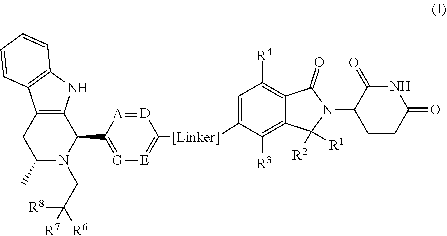
or a pharmaceutically acceptable salt thereof, wherein:
A and G are independently CR⁵ or N;
D and E are independently CH or N;
R¹ is H;
R² is H;
or R¹ and R² together with the carbon to which they are attached form carbonyl;
R³ is H or OMe;
R⁴ is H or OMe;
R⁵ is independently selected from H, F, Cl, CN, Me or OMe;
R⁶ is H, Me or F;
R⁷ is H, Me or F;
or R⁶ and R⁷ taken together with the carbon atom to which they are attached form a cyclopropyl ring or an oxetanyl ring;
R⁸ is H, Me, F, CH₂F, CHF₂, CF₃, CN, CH₂CN, CH₂OMe, CH₂OH, C(O)OH, C(O)OMe or SO₂Me;
Linker is an optionally substituted linking moiety comprising a branched or unbranched, cyclized or uncyclized, saturated or unsaturated chain of 6 to 15 carbon atoms in length, wherein 1 to 6 of the carbon atoms are optionally replaced with a heteroatom independently selected from O, N and S.

2. The compound of Formula (I), or a pharmaceutically acceptable salt thereof, as claimed in claim 1, wherein the Linker is a C₃₋₁₄ alkylene chain wherein one to four —CH₂-units in the alkylene chain may independently optionally be replaced with a group selected from —O—, —NH—, —NMe-, cycloalkyl, heterocycloalkyl, aryl and heteroaryl.

3. The compound of Formula (I), or a pharmaceutically acceptable salt thereof, as claimed in any preceding claim, wherein the Linker is a C₃₋₁₄ alkylene chain wherein one to four —CH₂— units in the alkylene chain are optionally replaced with a group independently selected from —O—, —NMe-, cycloalkyl and heterocycloalkyl.

4. The compound of Formula (I), or a pharmaceutically acceptable salt thereof, as claimed in any preceding claim, wherein Linker is represented by the moiety —X—[W]ₚ-Het¹-, wherein:
X is selected from the group consisting of -Het²-C₁₋₆alkylene-, —C(O)—Het²-C₁₋₆alkylene-, -Het²-C(O)—C₁₋₆ alkylene-, —C₁₋₆alkenylene-, —O-Het²-C₁₋₆alkylene-, —C₁₋₆alkylene- and —O-Cyc-C₁₋₆alkylene, wherein one or two —CH₂— units in the alkylene chain is independently replaced with —O—, —NH— or —NMe-;
W is selected from -Het³-C₁₋₆ alkylene-;
Het¹ is a nitrogen containing monocyclic or bicyclic heterocycloalkyl group;
Het² is a nitrogen containing monocyclic or bicyclic heterocycloalkyl group;
Het³ is a nitrogen containing monocyclic or bicyclic heterocycloalkyl group;
Cyc is C₃₋₆cycloalkyl;
p is 0 or 1;
wherein heterocycloalkyl is optionally substituted with 1 or 2 oxo substituents.

5. The compound of Formula (I), or a pharmaceutically acceptable salt thereof, as claimed in claim 4, wherein Het¹ is selected from the group consisting of piperidin-1-yl, piperazin-1-yl, 3,9-diazaspiro[5.5]undecan-3-yl, 7-oxa-3,10-diazaspiro[5.6]dodecan-3-yl, 3-oxopiperazin-1-yl, 2,7-diazaspiro[3.5]nonan-7-yl, 2,6-diazaspiro[3.3]heptan-2-yl, azetidin-1-yl and 2,5-diazabicyclo[2.2.1]heptan-2-yl.

6. The compound of Formula (I), or a pharmaceutically acceptable salt thereof, as claimed in claim 4 or claim 5, wherein Het² is selected from the group consisting of piperidin-4-yl, 3,9-diazaspiro[5.5]undecan-3-yl, 7-oxa-3,10-diazaspiro[5.6]dodecan-10-yl, 7-azaspiro[3.5]nonan-2-yl, 2-oxo-3,9-diazaspiro[5.5]undecan-3-yl, 2,7-diazaspiro[3.5]nonan-2-yl, 6-azaspiro[2.5]octan-1-yl, azetidin-3-yl and 3-azaspiro[5.5]undecan-3-yl.

7. The compound of Formula (I), or a pharmaceutically acceptable salt thereof, as claimed in any one of claims 4 to 6, wherein Het³ is selected from the group consisting of piperidin-4-yl, piperazin-1-yl and azetidin-1yl.

8. The compound of Formula (I), or a pharmaceutically acceptable salt thereof, as claimed in any one of claims 4 to 7, wherein Cyc is cyclobutyl.

9. The compound of Formula (I), or a pharmaceutically acceptable salt thereof, as claimed in any preceding claim, wherein Linker is selected from the group consisting of:

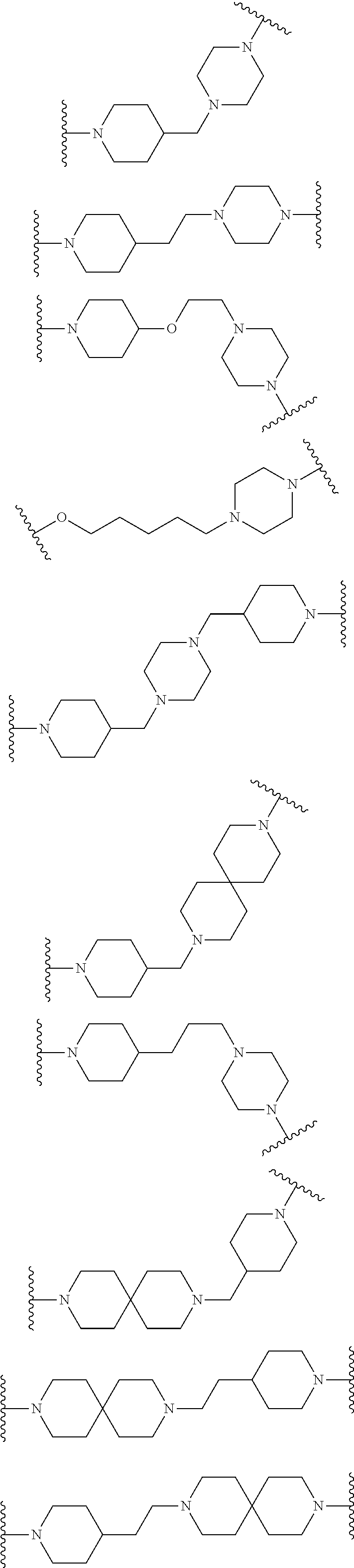

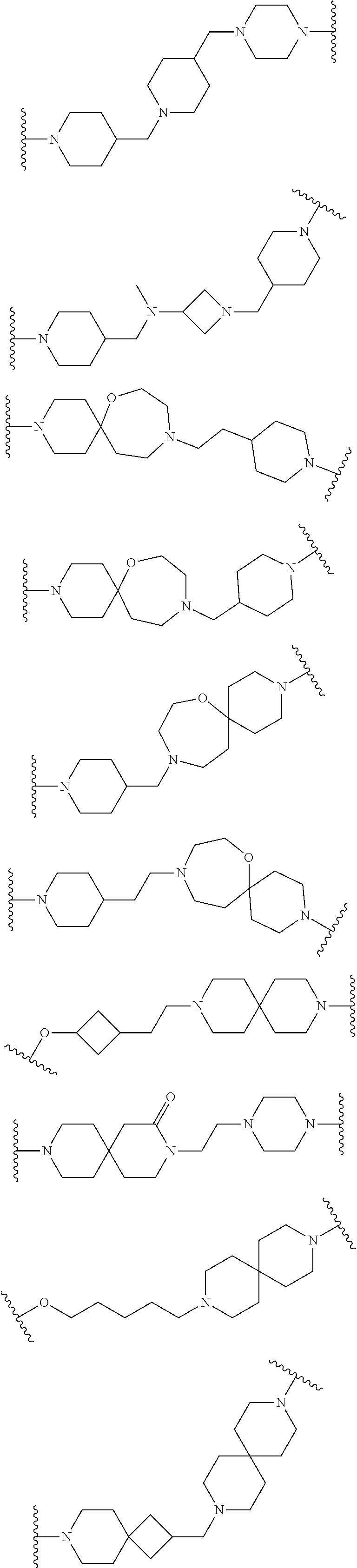

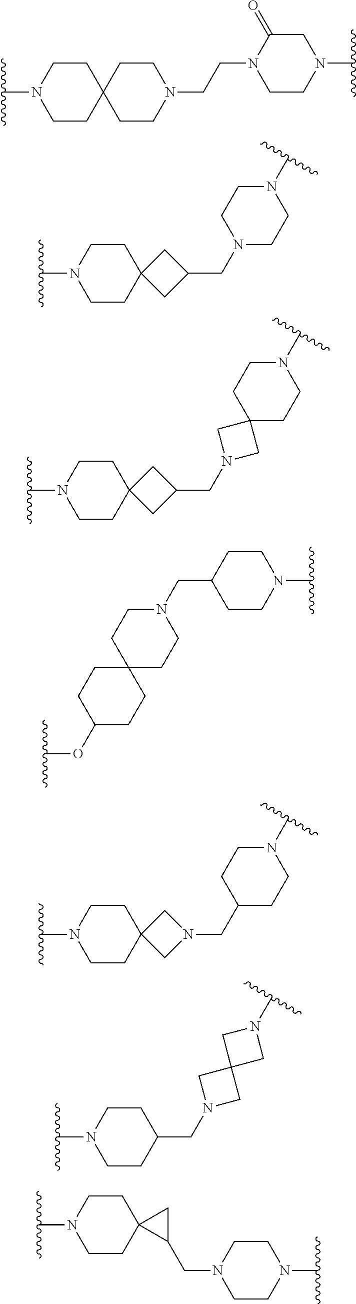

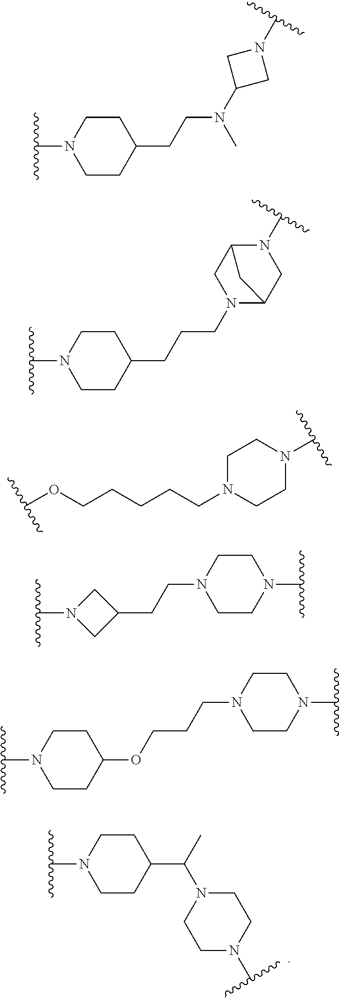 [SMILES: *N1CCC(CCN(C)C2CN(*)C2)CC1 ; *N1CCC(CCCN2CC3CC2CN3*)CC1 ; *OCCCCCN1CCN(*)CC1 ; *N1CCN(CCC2CN(*)C2)CC1 ; *N1CCC(OCCCN2CCN(*)CC2)CC1 ; *N1CCC(C(C)N2CCN(*)CC2)CC1]

10. The compound of Formula (I), or a pharmaceutically acceptable salt thereof, as claimed in any preceding claim, wherein the moiety: 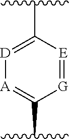
is selected from the group consisting of

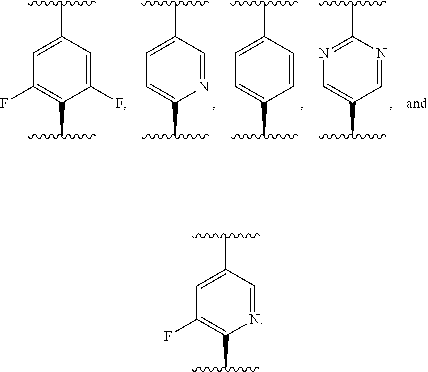

11. The compound of Formula (I), or a pharmaceutically acceptable salt thereof, as claimed in any preceding claim, wherein the moiety: 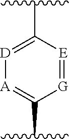
is selected from the group consisting of

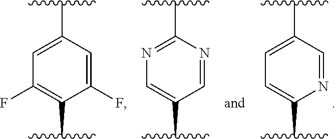

12. The compound of Formula (I), or a pharmaceutically acceptable salt thereof, as claimed in any preceding claim, wherein the group —CH₂—C(R⁶)(R⁷)(R⁸) is selected from the group consisting of:

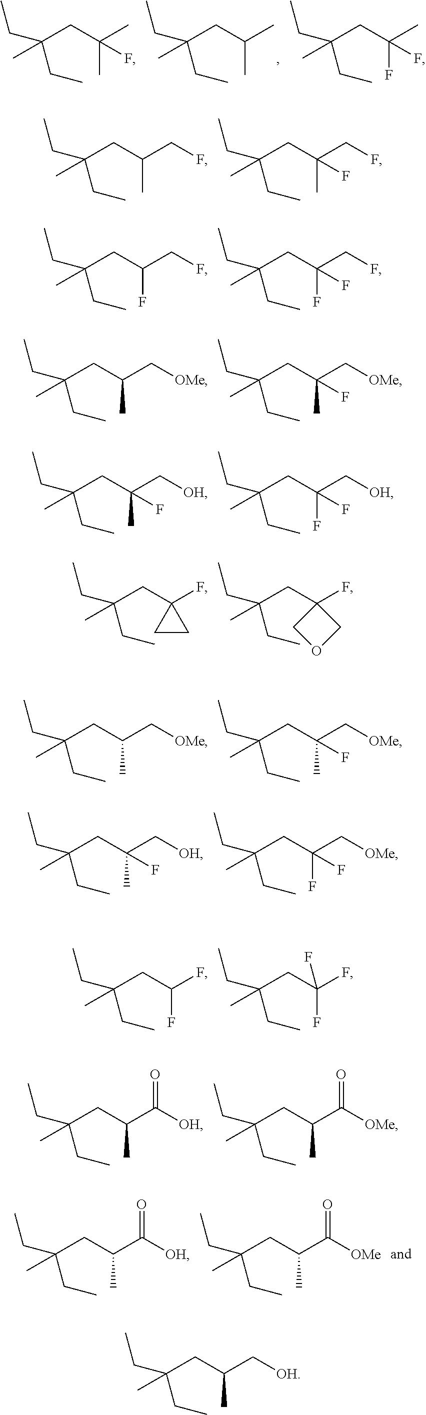

13. The compound of Formula (I), or a pharmaceutically acceptable salt thereof, as claimed in any preceding claim, wherein the group —CH₂—C(R⁶)(R⁷)(R⁸) is selected from the group consisting of:

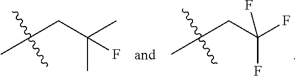

14. The compound of Formula (I), or a pharmaceutically acceptable salt thereof, as claimed in any preceding claim, wherein R³ and R⁴ are both H or one of R³ or R⁴ is OMe and the other is H.

15. The compound of Formula (I), or a pharmaceutically acceptable salt thereof, as claimed in any preceding claim, wherein R¹ and R² are both H.

16. The compound of Formula (I), or a pharmaceutically acceptable salt thereof, as claimed in claim 1, wherein the compound is selected from the group consisting of:
3-[5-[4-[[1-[5-[(1R,3R)-2-(2-Fluoro-2-methyl-propyl)-3-methyl-1,3,4,9-tetrahydropyrido[3,4-b]indol-1-yl]pyrimidin-2-yl]-4-piperidyl]methyl]piperazin-1-yl]-1-oxo-isoindolin-2-yl]piperidine-2,6-dione;
3-[5-[4-[2-[1-[5-[(1R,3R)-2-(2-Fluoro-2-methyl-propyl)-3-methyl-1,3,4,9-tetrahydropyrido[3,4-b]indol-1-yl]pyrimidin-2-yl]-4-piperidyl]ethyl]piperazin-1-yl]-1-oxo-isoindolin-2-yl]piperidine-2,6-dione;
2-[2,6-Dioxo3-piperidyl]-5-[4-[[1-[5-[(1R,3R)-2-(2-fluoro-2-methyl-propyl)-3-methyl-1,3,4,9-tetrahydropyrido[3,4-b]indol-1-yl]pyrimidin-2-yl]-4-piperidyl]methyl]piperazin-1-yl]isoindoline-1,3-dione formate;
3-[5-[4-[2-[[1-[5-[(1R,3R)-2-(2-Fluoro-2-methyl-propyl)-3-methyl-1,3,4,9-tetrahydropyrido[3,4-b]indol-1-yl]pyrimidin-2-yl]-4-piperidyl]oxy]ethyl]piperazin-1-yl]-1-oxo-isoindolin-2-yl]piperidine-2,6-dione;
3-[5-[4-[5-[3,5-Difluoro-4-[(1R,3R)-2-(2-fluoro-2-methyl-propyl)-3-methyl-1,3,4,9-tetrahydropyrido[3,4-b]indol-1-yl]phenoxy]pentyl]piperazin-1-yl]-1-oxo-isoindolin-2-yl]piperidine-2,6-dione;
3-{5-[4-({4-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]piperazin-1-yl}methyl)piperidin-1-yl]-1-oxo-1,3-dihydro-2H-isoindol-2-yl}piperidine-2,6-dione;
3-(5-{9-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]-3,9-diazaspiro[5.5]undecan-3-yl}-1-oxo-1,3-dihydro-2H-isoindol-2-yl)piperidine-2,6-dione;
3-(5-{4-[3-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)propyl]piperazin-1-yl}-1-oxo-1,3-dihydro-2H-isoindol-2-yl)piperidine-2,6-dione;
3-(5-{4-[(9-{5-[(1R,3R)-2-(2-fluoro-2-methylpropyl)-3-methyl-2,3,4,9-tetrahydro-1H-beta-carbolin-1-yl]pyrimidin-2-yl}-3,9-diazaspiro[5.5]undecan-3-yl)methyl]piperidin-1-yl}-1-oxo-1,3-dihydro-2H-isoindol-2-yl)piperidine-2,6-dione;
3-(5-{4-[2-(9-{5-[(1R,3R)-2-(2-fluoro-2-methylpropyl)-3-methyl-2,3,4,9-tetrahydro-1H-beta-carbolin-1-yl]pyrimidin-2-yl}-3,9-diazaspiro[5.5]undecan-3-yl)ethyl]piperidin-1-yl}-1-oxo-1,3-dihydro-2H-isoindol-2-yl)piperidine-2,6-dione;
3-(5-{9-[2-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl]-3,9-diazaspiro[5.5]undecan-3-yl}-1-oxo-1,3-dihydro-2H-isoindol-2-yl)piperidine-2,6-dione;
3-(5-{4-[2-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl]piperazin-1-yl}-7-methoxy-1-oxo-1,3-dihydro-2H-isoindol-2-yl)piperidine-2,6-dione;
3-(5-{4-[3-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)propyl]piperazin-1-yl}-7-methoxy-1-oxo-1,3-dihydro-2H-isoindol-2-yl)piperidine-2,6-dione;
3-{5-[4-({1-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]piperidin-4-yl}methyl)piperazin-1-yl]-1-oxo-1,3-dihydro-2H-isoindol-2-yl}piperidine-2,6-dione;
3-(5-{4-[2-(1-{5-[(1R,3R)-3-methyl-2-(2,2,2-trifluoroethyl)-2,3,4,9-tetrahydro-1H-beta-carbolin-1-yl]pyrimidin-2-yl}piperidin-4-yl)ethyl]piperazin-1-yl}-1-oxo-1,3-dihydro-2H-isoindol-2-yl)piperidine-2,6-dione;
3-(5-{4-[(3-{[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl](methyl)amino}azetidin-1-yl)methyl]piperidin-1-yl}-1-oxo-1,3-dihydro-2H-isoindol-2-yl)piperidine-2,6-dione;
3-(5-{4-[2-(3-{5-[(1R,3R)-2-(2-fluoro-2-methylpropyl)-3-methyl-2,3,4,9-tetrahydro-1H-beta-carbolin-1-yl]pyrimidin-2-yl}-7-oxa-3,10-diazaspiro[5.6]dodecan-10-yl)ethyl]piperidin-1-yl}-1-oxo-1,3-dihydro-2H-isoindol-2-yl)piperidine-2,6-dione;
3-(5-{4-[(3-{5-[(1R,3R)-2-(2-fluoro-2-methylpropyl)-3-methyl-2,3,4,9-tetrahydro-1H-beta-carbolin-1-yl]pyrimidin-2-yl}-7-oxa-3,10-diazaspiro[5.6]dodecan-10-yl)methyl]piperidin-1-yl}-1-oxo-1,3-dihydro-2H-isoindol-2-yl)piperidine-2,6-dione;
3-(5-{10-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]-7-oxa-3,10-diazaspiro[5.6]dodecan-3-yl}-1-oxo-1,3-dihydro-2H-isoindol-2-yl)piperidine-2,6-dione;
3-(5-{10-[2-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl]-7-oxa-3,10-diazaspiro[5.6]dodecan-3-yl}-1-oxo-1,3-dihydro-2H-isoindol-2-yl)piperidine-2,6-dione;
3-(5-{9-[(1-{6-[(1S,3R)-2-(2-fluoro-2-methylpropyl)-3-methyl-2,3,4,9-tetrahydro-1H-beta-carbolin-1-yl]pyridin-3-yl}piperidin-4-yl)methyl]-3,9-diazaspiro[5.5]undecan-3-yl}-1-oxo-1,3-dihydro-2H-isoindol-2-yl)piperidine-2,6-dione;
3-(5-{9-[(7-{5-[(1R,3R)-2-(2-fluoro-2-methylpropyl)-3-methyl-2,3,4,9-tetrahydro-1H-beta-carbolin-1-yl]pyrimidin-2-yl}-7-azaspiro[3.5]nonan-2-yl)methyl]-3,9-diazaspiro[5.5]undecan-3-yl}-1-oxo-1,3-dihydro-2H-isoindol-2-yl)piperidine-2,6-dione;
3-[5-(9-{2-[(1S,3r)-3-({5-[(1R,3R)-2-(2-fluoro-2-methylpropyl)-3-methyl-2,3,4,9-tetrahydro-1H-beta-carbolin-1-yl]pyrimidin-2-yl}oxy)cyclobutyl]ethyl}-3,9-diazaspiro[5.5]undecan-3-yl)-1-oxo-1,3-dihydro-2H-isoindol-2-yl]piperidine-2,6-dione;
3-(5-{9-[5-({5-[(1R,3R)-2-(2-fluoro-2-methylpropyl)-3-methyl-2,3,4,9-tetrahydro-1H-beta-carbolin-1-yl]pyrimidin-2-yl}oxy)pentyl]-3,9-diazaspiro[5.5]undecan-3-yl}-1-oxo-1,3-dihydro-2H-isoindol-2-yl)piperidine-2,6-dione;
3-(5-{4-[2-(9-{5-[(1R,3R)-2-(2-fluoro-2-methylpropyl)-3-methyl-2,3,4,9-tetrahydro-1H-beta-carbolin-1-yl]pyrimidin-2-yl}-2-oxo-3,9-diazaspiro[5.5]undecan-3-yl)ethyl]piperazin-1-yl}-1-oxo-1,3-dihydro-2H-isoindol-2-yl)piperidine-2,6-dione;
3-(5-{4-[2-(9-{5-[(1R,3R)-2-(2-fluoro-2-methylpropyl)-3-methyl-2,3,4,9-tetrahydro-1H-beta-carbolin-1-yl]pyrimidin-2-yl}-3,9-diazaspiro[5.5]undecan-3-yl)ethyl]-3-oxopiperazin-1-yl}-1-oxo-1,3-dihydro-2H-isoindol-2-yl)piperidine-2,6-dione;
3-(5-{4-[(7-{5-[(1R,3R)-2-(2-fluoro-2-methylpropyl)-3-methyl-2,3,4,9-tetrahydro-1H-beta-carbolin-1-yl]pyrimidin-2-yl}-7-azaspiro[3.5]nonan-2-yl)methyl]piperazin-1-yl}-1-oxo-1,3-dihydro-2H-isoindol-2-yl)piperidine-2,6-dione;
3-(5-{2-[(7-{5-[(1R,3R)-2-(2-fluoro-2-methylpropyl)-3-methyl-2,3,4,9-tetrahydro-1H-beta-carbolin-1-yl]pyrimidin-2-yl}-7-azaspiro[3.5]nonan-2-yl)methyl]-2,7-diazaspiro[3.5]nonan-7-yl}-1-oxo-1,3-dihydro-2H-isoindol-2-yl)piperidine-2,6-dione;
3-(5-{4-[(7-{5-[(1R,3R)-2-(2-fluoro-2-methylpropyl)-3-methyl-2,3,4,9-tetrahydro-1H-beta-carbolin-1-yl]pyrimidin-2-yl}-2,7-diazaspiro[3.5]nonan-2-yl)methyl]piperidin-1-yl}-1-oxo-1,3-dihydro-2H-isoindol-2-yl)piperidine-2,6-dione;
3-(5-{6-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]-2,6-diazaspiro[3.3]heptan-2-yl}-1-oxo-1,3-dihydro-2H-isoindol-2-yl)piperidine-2,6-dione;
3-(5-{4-[(6-{5-[(1R,3R)-2-(2-fluoro-2-methylpropyl)-3-methyl-2,3,4,9-tetrahydro-1H-beta-carbolin-1-yl]pyrimidin-2-yl}-6-azaspiro[2.5]octan-1-yl)methyl]piperazin-1-yl}-1-oxo-1,3-dihydro-2H-isoindol-2-yl)piperidine-2,6-dione;
3-[5-(3-{[2-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl](methyl)amino}azetidin-1-yl)-1-oxo-1,3-dihydro-2H-isoindol-2-yl]piperidine-2,6-dione;
3-(5-{(1R,4R)-5-[3-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)propyl]-2,5-diazabicyclo[2.2.1]heptan-2-yl}-1-oxo-1,3-dihydro-2H-isoindol-2-yl)piperidine-2,6-dione;
3-(5-{4-[3-(1-{5-[(1R,3R)-3-methyl-2-(2,2,2-trifluoroethyl)-2,3,4,9-tetrahydro-1H-beta-carbolin-1-yl]pyrimidin-2-yl}piperidin-4-yl)propyl]piperazin-1-yl}-1-oxo-1,3-dihydro-2H-isoindol-2-yl)piperidine-2,6-dione;
3-(5-{4-[(1-{5-[(1R,3R)-3-methyl-2-(2,2,2-trifluoroethyl)-2,3,4,9-tetrahydro-1H-beta-carbolin-1-yl]pyrimidin-2-yl}piperidin-4-yl)methyl]piperazin-1-yl}-1-oxo-1,3-dihydro-2H-isoindol-2-yl)piperidine-2,6-dione;
3-(5-{4-[1-(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)ethyl]piperazin-1-yl}-1-oxo-1,3-dihydro-2H-isoindol-2-yl)piperidine-2,6-dione;
3-(5-{4-[2-(1-{5-[(1R,3R)-2-(2-fluoro-2-methylpropyl)-3-methyl-2,3,4,9-tetrahydro-1H-beta-carbolin-1-yl]pyrimidin-2-yl}azetidin-3-yl)ethyl]piperazin-1-yl}-1-oxo-1,3-dihydro-2H-isoindol-2-yl)piperidine-2,6-dione;
3-[5-(4-{3-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)oxy]propyl}piperazin-1-yl)-1-oxo-1,3-dihydro-2H-isoindol-2-yl]piperidine-2,6-dione;
3-(5-{4-[(1-{5-[(1R,3R)-2-(2-fluoro-2-methylpropyl)-3-methyl-2,3,4,9-tetrahydro-1H-beta-carbolin-1-yl]pyrimidin-2-yl}piperidin-4-yl)methyl]piperazin-1-yl}-7-methoxy-1-oxo-1,3-dihydro-2H-isoindol-2-yl)piperidine-2,6-dione;
3-(5-{4-[5-({5-[(1R,3R)-2-(2-fluoro-2-methylpropyl)-3-methyl-2,3,4,9-tetrahydro-1H-beta-carbolin-1-yl]pyrimidin-2-yl}oxy)pentyl]piperazin-1-yl}-1-oxo-1,3-dihydro-2H-isoindol-2-yl)piperidine-2,6-dione; and
3-[5-(4-{[9-({5-[(1R,3R)-2-(2-fluoro-2-methylpropyl)-3-methyl-2,3,4,9-tetrahydro-1H-beta-carbolin-1-yl]pyrimidin-2-yl}oxy)-3-azaspiro[5.5]undecan-3-yl]methyl}piperidin-1-yl)-1-oxo-1,3-dihydro-2H-isoindol-2-yl]piperidine-2,6-dione.

17. A pharmaceutical composition comprising a compound of Formula (I), or a pharmaceutically acceptable salt thereof, as claimed in any preceding claim, and at least one pharmaceutically acceptable excipient.

18. A compound of Formula (I), or a pharmaceutically acceptable salt thereof, as claimed in any one of claims 1 to 16, for use in therapy.

19. A compound of Formula (I), or a pharmaceutically acceptable salt thereof, as claimed in any one of claims 1 to 16, for use in the treatment of cancer.

20. Use of a compound of Formula (I), or a pharmaceutically acceptable salt thereof, as claimed in any one of claims 1 to 16, for the manufacture of a medicament for the treatment of cancer.

21. A method for the treatment of cancer in a warm-blooded animal in need of such treatment, wherein the method comprises administering to said warm-blooded animal a therapeutically effective amount of a compound of Formula (I), or a pharmaceutically acceptable salt thereof, as claimed in any one of claims 1 to 16.

22. The compound for use, use or method as claimed in any one of claims 19 to 21, wherein the cancer is selected from breast, endometrium, ovary and cervix.
